(19) United States
(12) Patent Application Publication
(10) Pub. No.: US 2025/0129047 A1
(43) Pub. Date: Apr. 24, 2025

(54) ISOQUINOLINE DERIVATIVES AS MUTANT EGFR MODULATORS AND USES THEREOF

(71) Applicant: NEWAVE PHARMACEUTICAL INC., Pleasanton, CA (US)

(72) Inventor: Yi Chen, Pleasanton, CA (US)

(21) Appl. No.: 18/572,330

(22) PCT Filed: Jul. 1, 2022
(86) PCT No.: PCT/US2022/035951

§ 371 Date: Dec. 20, 2023

Related U.S. Application Data
(60) Provisional application No. 63/314,390, filed on Feb. 26, 2022.
(60) Provisional application No. 63/277,898, filed on Nov. 10, 2021.
(60) Provisional application No. 63/218,363, filed on Jul. 4, 2021.

(51) Int. Cl. C07D 401/14; A61K 31/4725

(52) U.S. Cl. CPC C07D 401/14; A61K 31/4725

(57) ABSTRACT

The disclosure includes compounds of Formula (1), wherein each of R_{A}, R_{B}, R₁, R₂, R₃, R₄, Warhead, Z, Q₄, W¹, W², m, n, i, and j, are defined herein. Also disclosed is a method for treating a neoplastic disease, autoimmune disease, and inflammatory disorder with these compounds.

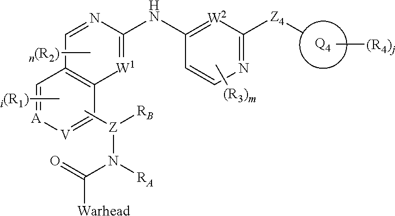

### REFERENCE TO RELATED APPLICATIONS

This International Patent Application claims priority to and the benefit of the filing date of U.S. Provisional Application Nos. 63/218,363, filed on Jul. 4, 2021, 63/277,898, filed on Nov. 10, 2021, and 63/314,390, filed on Feb. 26, 2022. The entire contents of each of the foregoing applications are expressly incorporated herein by reference.

### BACKGROUND OF THE INVENTION

The HER family receptor tyrosine kinases are mediators of cell growth, differentiation, and survival. The receptor family includes four distinct members, i.e. epidermal growth factor receptor (EGFR, ErbBl, or HER1), HER2 (ErbB2), HER3 (ErbB3) and HER4 (ErbB4). Upon ligand binding, the receptors form homo and heterodimers and subsequent activation of the intrinsic tyrosine kinase activity leads to receptor auto-phosphorylation and the activation of downstream signaling molecules (Yarden, Y., Sliwkowski, MX. Untangling the ErbB signaling network. Nature Review Mol Cell Biol. 2001 February; 2(2): 127-37). These signaling molecules promote cell growth and proliferation. Deregulation of EGFR by overexpression or mutation has been implicated in many types of human cancer including colorectal, pancreatic, gliomas, head and neck and lung cancer, in particular non-small cell lung cancer (NSCLC). Several EGFR targeting agents have been developed over the years (Ciardiello, F., and Tortora, G. (2008). EGFR antagonists in cancer treatment. The New England journal of medicine 358, 1160-1174). Erlotinib (Tarceva®), a reversible inhibitor of the EGFR tyrosine kinase is approved in numerous countries for the treatment of recurrent NSCLC.

An impressive single agent activity of EGFR tyrosine kinase inhibitors is observed in a subset of NSCLC patients whose tumors harbor somatic kinase domain mutations, whereas clinical benefit in wild-type EGFR patients is greatly diminished (Paez, J. et al. (2004). EGFR mutations in lung cancer: correlation with clinical response to gefitinib therapy. Science (New York, NY 304, 1497-1500). The most common somatic mutations of EGFR are exon 19 deletions with delta 746-750 the most prevalent mutation and the exon 21 amino acid substitutions with L858R the most frequent mutation (Sharma S V, Bell D W, Settleman J, Haber D A. Epidermal growth factor receptor mutations in lung cancer. Nat Rev Cancer. 2007 March; 7(3): 169-81).

Treatment resistance arises frequently, often due to the secondary T790M mutation within the ATP site of the receptor. Some developed mutant-selective irreversible inhibitors are highly active against the T790M mutant, but their efficacy can be compromised by acquired mutation of C797S, that is the cysteine residue with which they form a key covalent bond (Thress, K. S. et al. Acquired EGFR C797S mutation mediates resistance to AZD9291 in non-small cell lung cancer harboring EGFR T790M. Nat. Med. 21, 560-562 (2015)). C797S mutation was further reported by Wang to be a major mechanism for resistance to T790M-targeting EGFR inhibitors (Wang et al. EGFR C797S mutation mediates resistance to third-generation inhibitors in T790M-positive non-small cell lung cancer, J Hematol Oncol. 2016; 9: 59). Additional mutations that cause resistance to Osimertinib are described by Yang, for example L718Q. (Yang et al, Investigating Novel Resistance Mechanisms to Third-Generation EGFR Tyrosine Kinase Inhibitor Osimertinib in Non-Small Cell Lung Cancer Patients, Clinical Cancer Research, DOI: 10.1158/1078-0432.CCR-17-2310). Additional mutations targeting strategies are also known including Targeting EGFRL858R/T790M and EGFRL858R/T790M/C797S resistance mutations in NSCLC treatment (Lu et al. Targeting EGFR^{L858R/T790M} and EGFR^{L858R/T790M/C797S} resistance mutations in NSCLC: Current developments in medicinal chemistry, Med Res Rev 2018; 1-32).

Additional examples of EGFR inhibitors, in particular selective inhibitors of T790M containing EGFR mutants, have also been described including those described in WO2014081718, WO2014210354, WO2018/115218, WO2018220149, WO2020002487, and ZHOU et al., “Novel mutant-selective EGFR kinase inhibitors against EGFR T790M”, NATURE, (20091224), vol. 462, no. 7276, doi:10.1038/nature08622, ISSN 0028-0836, pages 1070-1074.

The field of targeted protein degradation promoted by small molecules has been intensively studied (Collins et al., Biochem J, 2017, 474(7), 1127-47). Protein degradation plays a role in various cellular functions. For example, the body uses protein degradation to adjust the concentrations of regulatory proteins through degradation into small peptides to maintain health and productivity of the cells.

Cereblon is a protein that forms an E3 ubiquitin ligase complex, which ubiquitinates various other proteins. Cereblon is known as the primary target for the anticancer thalidomide analogs. A higher expression of cereblon has been linked to the efficiency of thalidomide analogs in cancer therapy.

Compounds have been described as useful modulators of targeted ubiquitination, for example the compounds described in. WO2013020557, WO2013063560, WO2013106643, WO/2013170147, WO2016011906, and WO/2019183523 can be used for targeted ubiquitination. Additional modulators for targeted ubiquitination include those described by Ranok Therapeutics Hangzhou WO2020206608 and WO2020207396; those described by Arvinas in WO2015160845, WO2016149668, WO2016197032, WO2017011590, WO2017030814, WO2018144649, WO2018226542, and WO2019199816; those described by Dana-Farber Cancer Institute in WO2016105518, WO2017007612, WO2017024317, WO2017024318, WO2017117473, WO2017117474, WO2018148443, WO2018148440, and WO2019165229; those described by Kymera in WO2019/060742, WO2019/140387, and WO2020/01022; and those described by C4 Therapeutics Inc. in WO2017197036, WO2017197046, WO2017197051, WO2017197055, WO2018237026, WO2019099868, WO2019191112, WO2019204353, WO2019236483, WO2020132561, WO2020181232, and WO2020210630.

Some specific molecules for the degradation of EGFR have also been described, for example, Dana-Farber Cancer Institute described EGFR degraders in WO2017185036. F. Hoffman-La-Roche described EGFR degraders in WO2019121562 and WO2019149922. Arvinas has described EGFR degraders in WO2018119441.

Despite these efforts, there remains a need for new EGFR modulators to treat disorders mediated by EGFR in hosts, and in particular humans, in need thereof.

### SUMMARY OF THE INVENTION

In one aspect, this invention relates to a compound of Formula (1), or an N-oxide thereof, or a pharmaceutically acceptable salt, solvate, polymorph, tautomer, stereoisomer, an isotopic form, or a prodrug of said compound of Formula (1) or N-oxide thereof:

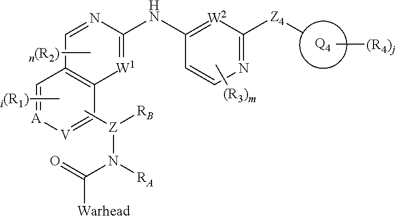

wherein
Warhead is chemical group that can form a covalent bond with the thio group of Cys797 in EGFR;
W¹ is CH and W² is N; or W¹ is N and W² is CH;
each of A and V, independently, is N or CH; wherein R₁ or Z can be linked to A or Z when A or Z is CH;
Q₄, is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{d};
Z is absent, alkyl, alkenyl, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, or bridged-heterocyclic, each of which is independently optionally substituted with one or more R_{d};
Z₄ is a bond, (CRₐR_{b})ₚ, N(Rₐ), O, S, C(O), S(O₂), —O(CRₐR_{b})ₚ—, —N(Rₐ)(CRₐR_{b})ₚ, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, (CRₐR_{b})ₚC(O)N(Rₐ)(CRₐR_{b})_{q}, a bivalent alkenyl group, or a bivalent alkynyl group;
each of R_{A}, R_{B}, R₁, R₂, R₃, or R₄, independently, is absent, H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{d};
Rₐ, R_{b}, R_{c} and R_{d}, independently, is H, D, alkyl, alkenyl, alkynyl, spiroalkyl, halo, cyano, amine, nitro, hydroxy, ═O, -alkyl-O—P(O)(OH)(OH), C(O)NHOH, C(O)OH, C(O)NH₂, —SO₂Rₑ, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more Rₐ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, —SO₂R_{f}, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{f};
R_{f} is H, D, alkyl, alkenyl, alkynyl, spiroalkyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{g};
R_{g} is H, D, alkyl, alkenyl, alkynyl, spiroalkyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
R_{A} and R_{B} groups, taken together with the atom to which they are attached, may optionally form a heterocycloalkyl optionally substituted with one or more R_{d};
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
two of R_{d} groups, taken together with the atom to which they are attached, may optionally form a cycloalkyl or heterocycloalkyl optionally substituted with one or more Rₐ;
two of Rₐ groups, taken together with the atom to which they are attached, may optionally form a cycloalkyl or heterocycloalkyl optionally substituted with one or more R_{f};
each of m, n, and i, independently, is 0, 1, 2, 3, 4, 5, or 6; and
j is 0, 1, 2, 3, 4, 5, or 6.
   In preferred embodiments, the compound is represented by Formula (2):

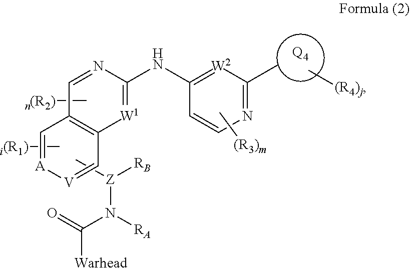

wherein
Warhead is

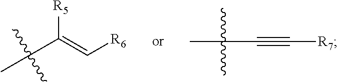

and
each of R₅, R₆, or R₇, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, heteroaryl, each of which is independently optionally substituted with one or more R_{d}.

The remaining groups are as defined in Formula (1).

In preferred embodiment, the compound is represented by Formula (3)

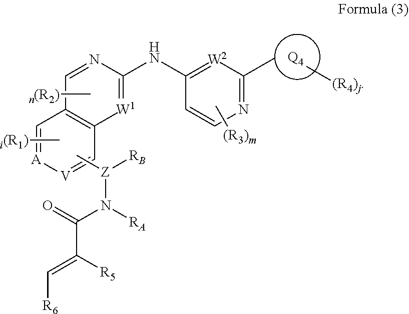

The remaining groups are as defined in Formula (1).

In preferred embodiments, the compound is represented by Formula (4):

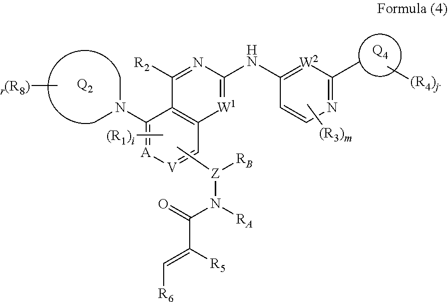

wherein
Q₂, is a heterocycloalkyl, heterocycloalkenyl, spiro-heterocyclic, fused-heterocyclic, or bridged-heterocyclic, each of which is independently optionally substituted with one or more R_{d};
each of R₈, independently, is absent, H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, heteroaryl, each of which is independently optionally substituted with one or more Rₐ;
r is 0, 1, 2, 3, 4, 5, or 6; and
i is 0, 1, or 2.

The remaining groups are as defined in Formula (1).

In preferred embodiments, the compound is represented by Formula (5):

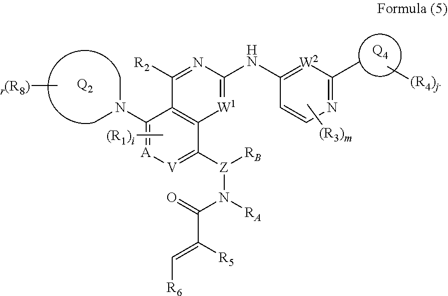

The remaining groups are as defined in Formula (1).
In preferred embodiments, the compound is represented by Formula (6):

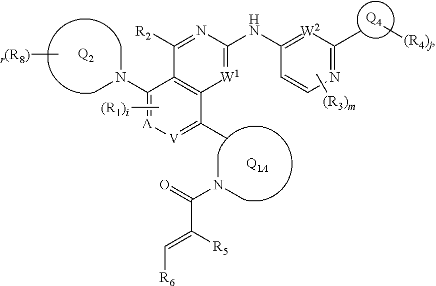

wherein
Q_{1A} is a heterocycloalkyl, heterocycloalkenyl, spiro-heterocyclic, fused-heterocyclic, or bridged-heterocyclic, each of which is independently optionally substituted with one or more R_{d}. The remaining groups are as defined in Formula (1).
   In preferred embodiments, the compound is represented by Formula (7):

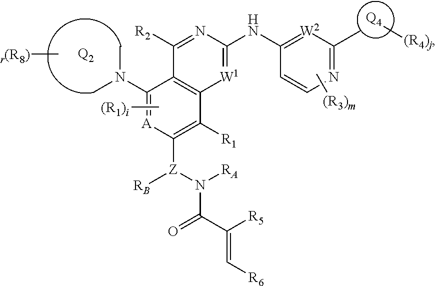

wherein
i is 0 or 1. The remaining groups are as defined in Formula (1).
   In preferred embodiments, the compound is represented by Formula (8):

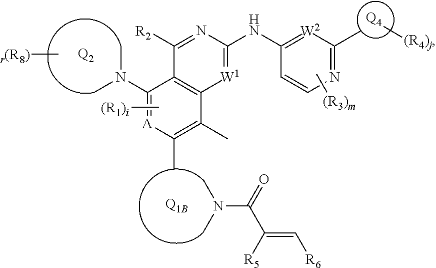

wherein

Q_{1B} is a heterocycloalkyl, heterocycloalkenyl, spiro-heterocyclic, fused-heterocyclic, or bridged-heterocyclic, each of which is independently optionally substituted with one or more R_{d}. The remaining groups are as defined in Formula (1).

Compounds of the invention may contain one or more asymmetric carbon atoms. Accordingly, the compounds may exist as diastereomers, enantiomers, or mixtures thereof. Each of the asymmetric carbon atoms may be in the R or S configuration, and both of these configurations are within the scope of the invention.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

It should be recognized that the compounds of the present invention may be present and optionally administered in the form of salts or solvates. The invention encompasses any pharmaceutically acceptable salts and solvates of any one of the above-described compounds and modifications thereof.

Also within the scope of this invention is a pharmaceutical composition containing one or more of the compounds, modifications, and/or salts and thereof described above. Further within the scope of the invention is a pharmaceutical composition of the invention for use in treating a neoplastic disease, autoimmune disease, and inflammatory disorders, therapeutic uses thereof, and use of the compounds for the manufacture of a medicament for treating the disease/disorder.

This invention also relates to a method of treating a neoplastic disease, by administering to a subject in need thereof an effective amount of one or more of the compounds, modifications, and/or salts, and compositions thereof described above.

Autoimmune and/or inflammatory diseases that can be affected using compounds and compositions according to the invention include, but are not limited to: psoriasis, allergy, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic Idiopathic thrombocytopenic purpura (ITP), Addison's disease, Parkinson's disease, Alzheimer's disease, diabetes, septic shock, and myasthenia gravis.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:
2-(2,6-dioxopiperidin-3-yl)-4-(((((2R, 3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)methyl)isoindoline-1,3-dione,
2-(2,6-dioxopiperidin-3-yl)-4-(2-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)ethyl)isoindoline-1,3-dione,
2-(2,6-dioxopiperidin-3-yl)-4-((2-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)ethyl) amino)isoindoline-1,3-dione,
2-(2,6-dioxopiperidin-3-yl)-4-((3-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)propyl)amino)isoindoline-1,3-dione,
2-(2,6-dioxopiperidin-3-yl)-4-((4-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)butyl) amino)isoindoline-1,3-dione,
2-(2,6-dioxopiperidin-3-yl)-4-((5-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)pentyl)amino)isoindoline-1,3-dione,
2-(2,6-dioxopiperidin-3-yl)-4-((6-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)hexyl)amino)isoindoline-1,3-dione,
2-(2,6-dioxopiperidin-3-yl)-5-(((((2R, 3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)methyl)isoindoline-1,3-dione,
2-(2,6-dioxopiperidin-3-yl)-5-(2-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)ethyl)isoindoline-1,3-dione,
2-(2,6-dioxopiperidin-3-yl)-5-((2-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)ethyl) amino)isoindoline-1,3-dione,
2-(2,6-dioxopiperidin-3-yl)-5-((3-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)propyl)amino)isoindoline-1,3-dione,
2-(2,6-dioxopiperidin-3-yl)-5-((4-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)butyl) amino)isoindoline-1,3-dione,
2-(2,6-dioxopiperidin-3-yl)-5-((5-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)pentyl)amino)isoindoline-1,3-dione,
2-(2,6-dioxopiperidin-3-yl)-5-((6-((((2R,3S)-1-(5-isopropyl-3-((2-(4-methoxypiperidin-1-yl)pyrimidin-4-yl)amino)isoquinolin-8-yl)-2-methylazetidin-3-yl)methyl)sulfonyl)hexyl)amino)isoindoline-1,3-dione,
2-(2,6-dioxopiperidin-3-yl)-4-(((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)sulfonyl)methyl)isoindoline-1,3-dione,
2-(2,6-dioxopiperidin-3-yl)-4-(2-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)sulfonyl)ethyl)isoindoline-1,3-dione,
2-(2,6-dioxopiperidin-3-yl)-4-((2-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)suIfonyl)ethyl)amino)isoindoline-1,3-dione,
2-(2,6-dioxopiperidin-3-yl)-4-((3-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)suIfonyl)propyl)amino)isoindoline-1,3-dione,
2-(2,6-dioxopiperidin-3-yl)-4-((4-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)suIfonyl)butyl)amino)isoindoline-1,3-dione,
2-(2,6-dioxopiperidin-3-yl)-4-((5-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)suIfonyl)pentyl)amino)isoindoline-1,3-dione,
2-(2,6-dioxopiperidin-3-yl)-4-((6-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)suIfonyl)hexyl)amino)isoindoline-1,3-dione,
2-(2,6-dioxopiperidin-3-yl)-5-(((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)sulfonyl)methyl)isoindoline-1,3-dione,
2-(2,6-dioxopiperidin-3-yl)-5-(2-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)sulfonyl)ethyl)isoindoline-1,3-dione,
2-(2,6-dioxopiperidin-3-yl)-5-((2-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)suIfonyl)ethyl)amino)isoindoline-1,3-dione,
2-(2,6-dioxopiperidin-3-yl)-5-((3-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)suIfonyl)propyl)amino)isoindoline-1,3-dione,
2-(2,6-dioxopiperidin-3-yl)-5-((4-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)suIfonyl)butyl)amino)isoindoline-1,3-dione,
2-(2,6-dioxopiperidin-3-yl)-5-((5-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)suIfonyl)pentyl)amino)isoindoline-1,3-dione,
S
2-(2,6-dioxopiperidin-3-yl)-5-((6-((((2R,3S)-1-(3-((2-((3S,4R)-3-fluoro-4-hydroxy-3-methylpiperidin-1-yl)pyrimidin-4-yl)amino)-5-isopropylisoquinolin-8-yl)-2-methylazetidin-3-yl)methyl)suIfonyl)hexyl)amino)isoindoline-1,3-dione,
N-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)acrylamide,
N-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)but-2-ynamide,
N-(3-((2-((3R,4R)-3-fluoro-4-methoxypiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-methoxypiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3R,4S)-3-fluoro-4-methoxypiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(5-isopropyl-3-((2-(4-methoxypiperidin-1-yl)pyrimidin-4-yl)amino)-8-((2R,3S)-2-methyl-3-((methylsulfonyl)methyl)azetidin-1-yl)isoquinolin-6-yl)acrylamide,
N-(5-isopropyl-3-((2-(4-methoxypiperidin-1-yl)pyrimidin-4-yl)amino)-8-((2R,3S)-2-methyl-3-((methylsulfonyl)methyl)azetidin-1-yl)isoquinolin-6-yl)but-2-ynamide,
N-(3-((2-(4-hydroxy-4-methylpiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-(hexahydro-1H-furo[3,4-b]pyrrol-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((1R,5S)-6-oxa-2-azabicyclo[3.2.1]octan-2-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((1 S,5R)-6-oxa-2-azabicyclo[3.2.1]octan-2-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-hydroxy-3-methylpiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-hydroxy-3-methylpiperidin-1-yl)pyrimidin-4-yl)amino)-5-isopropyl-8-((2S,3R)-2-methyl-3-((methylsulfonyl)methyl)azetidin-1-yl)isoquinolin-6-yl)acrylamide,
N-(3-((2-((3S,4R)-3-fluoro-4-hydroxy-3-methylpiperidin-1-yl)pyrimidin-4-yl)amino)-5-isopropyl-8-((2R,3S)-2-methyl-3-((methylsulfonyl)methyl)azetidin-1-yl)isoquinolin-6-yl)but-2-ynamide,
(E)-4-(dimethylamino)-N-(3-((2-((3S,4R)-3-fluoro-4-hydroxy-3-methylpiperidin-1-yl)pyrimidin-4-yl)amino)-5-isopropyl-8-((2R,3S)-2-methyl-3-((methylsulfonyl)methyl)azetidin-1-yl)isoquinolin-6-yl)but-2-enamide,
(E)-2-cyano-N-(3-((2-((3S,4R)-3-fluoro-4-hydroxy-3-methylpiperidin-1-yl)pyrimidin-4-yl)amino)-5-isopropyl-8-((2R,3S)-2-methyl-3-((methylsulfonyl)methyl)azetidin-1-yl)isoquinolin-6-yl)-4-methyl-4-(4-(oxetan-3-yl)piperazin-1-yl)pent-2-enamide,
N-(3-((2-((3R,4R)-3-fluoro-4-hydroxypiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
1-((R)-2-(5-isopropyl-3-((2-(4-methoxypiperidin-1-yl)pyrimidin-4-yl)amino)-8-((2R,3S)-2-methyl-3-((methylsulfonyl)methyl)azetidin-1-yl)isoquinolin-6-yl)azetidin-1-yl)prop-2-en-1-one,
1-((S)-2-(5-isopropyl-3-((2-(4-methoxypiperidin-1-yl)pyrimidin-4-yl)amino)-8-((2R,3S)-2-methyl-3-((methylsulfonyl)methyl)azetidin-1-yl)isoquinolin-6-yl)azetidin-1-yl)prop-2-en-1-one,
1-((S)-2-(5-isopropyl-3-((2-(4-methoxypiperidin-1-yl)pyrimidin-4-yl)amino)-8-((2R,3S)-2-methyl-3-((methylsulfonyl)methyl)azetidin-1-yl)isoquinolin-6-yl)pyrrolidin-1-yl)prop-2-en-1-one,
1-((R)-2-(5-isopropyl-3-((2-(4-methoxypiperidin-1-yl)pyrimidin-4-yl)amino)-8-((2R,3S)-2-methyl-3-((methylsulfonyl)methyl)azetidin-1-yl)isoquinolin-6-yl)pyrrolidin-1-yl)prop-2-en-1-one,
1-((S)-2-(5-isopropyl-3-((2-(4-methoxypiperidin-1-yl)pyrimidin-4-yl)amino)-8-((2R,3S)-2-methyl-3-((methylsulfonyl)methyl)azetidin-1-yl)isoquinolin-6-yl)piperidin-1-yl)prop-2-en-1-one,
1-((R)-2-(5-isopropyl-3-((2-(4-methoxypiperidin-1-yl)pyrimidin-4-yl)amino)-8-((2R,3S)-2-methyl-3-((methylsulfonyl)methyl)azetidin-1-yl)isoquinolin-6-yl)piperidin-1-yl)prop-2-en-1-one,
1-(3-(5-isopropyl-3-((2-(4-methoxypiperidin-1-yl)pyrimidin-4-yl)amino)-8-((2R, 3S)-2-methyl-3-((methylsulfonyl)methyl)azetidin-1-yl)isoquinolin-6-yl)azetidin-1-yl)prop-2-en-1-one,
1-(3-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)azetidin-1-yl)prop-2-en-1-one,
1-(3-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)azetidin-1-yl)but-2-yn-1-one,
1-((R)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)azetidin-1-yl)prop-2-en-1-one,
1-((S)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)azetidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)azetidin-1-yl)but-2-yn-1-one,
1-((S)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)azetidin-1-yl)but-2-yn-1-one,
1-((S)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)pyrrolidin-1-yl)but-2-yn-1-one,
1-((R)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)pyrrolidin-1-yl)but-2-yn-1-one,
1-((R)-3-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((S)-3-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-3-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)pyrrolidin-1-yl)but-2-yn-1-one,
1-((S)-3-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)pyrrolidin-1-yl)but-2-yn-1-one,
1-((S)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)piperidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)piperidin-1-yl)prop-2-en-1-one,
1-((S)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)piperidin-1-yl)but-2-yn-1-one,
1-((R)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)piperidin-1-yl)but-2-yn-1-one,
1-((R)-3-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)piperidin-1-yl)prop-2-en-1-one,
1-((S)-3-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)piperidin-1-yl)prop-2-en-1-one,
1-((R)-3-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)piperidin-1-yl)but-2-yn-1-one,
1-((S)-3-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)piperidin-1-yl)but-2-yn-1-one,
1-((S)-2-(3-((2-((3R,4S)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)azepan-1-yl)prop-2-en-1-one,
1-((R)-2-(3-((2-((3R,4S)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)azepan-1-yl)prop-2-en-1-one,
1-((S)-2-(3-((2-((3R,4S)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)azepan-1-yl)but-2-yn-1-one,
1-((R)-2-(3-((2-((3R,4S)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)azepan-1-yl)but-2-yn-1-one,
2-fluoro-1-((R)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
(E)-4-(dimethylamino)-1-((R)-2-(3-((2-((3S,4R)-3-fluoro-4-methoxypiperidin-1-yl)pyrimidin-4-yl)amino)-5-isopropyl-8-(3-((methylsulfonyl)methyl)azetidin-1-yl)isoquinolin-6-yl)pyrrolidin-1-yl)but-2-en-1-one,
1-((R)-2-(6-((2-((3S,4R)-3-fluoro-4-methoxypiperidin-1-yl)pyrimidin-4-yl)amino)-4-isopropyl-1-(3-((methylsulfonyl)methyl)azetidin-1-yl)-2,7-naphthyridin-3-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-((2R,3S)-2-methyl-3-((methylsulfonyl)methyl)azetidin-1-yl)isoquinolin-6-yl)pyrrolidin-1-yl)prop-2-en-1-one,
1-((R)-2-(6-((2-((3R,4S)-3-fluoro-4-methoxypiperidin-1-yl)pyrimidin-4-yl)amino)-4-isopropyl-1-(3-((methylsulfonyl)methyl)azetidin-1-yl)-2,7-naphthyridin-3-yl)pyrrolidin-1-yl)prop-2-en-1-one,
1-((R)-2-(3-((2-((2S,4S,5R)-5-fluoro-4-methoxy-2-methylpiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((2R,4S,5R)-5-fluoro-4-methoxy-2-methylpiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((2S,4R,5S)-5-fluoro-4-methoxy-2-methylpiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((2R,4R,5S)-5-fluoro-4-methoxy-2-methylpiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-isopropoxypi peridin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-4-(tert-butoxy)-3-fluoropiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
4R)-3-fluoro-4-(trifluoromethoxy)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-(2-hydroxyethoxy)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
4R)-3-fluoro-4-(2-methoxyethoxy)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-(2-(methyl amino)ethoxy)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-(3-morpholinopropoxy)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-((tetrahydro-2H-pyran-4-yl)oxy)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-((4-hydroxy-4-methylcyclohexyl)oxy)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-((1-(tetrahydro-2H-pyran-4-yl)piperidin-4-yl)oxy)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-((1-(6-(trifluoromethyl)pyridin-2-yl)piperidin-4-yl)oxy)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
5-(4-(((3S,4R)-1-(4-((6-((R)-1-acryloylpyrrolidin-2-yl)-5-isopropyl-8-(3-((methylsulfonyl)methyl)azetidin-1-yl)isoquinolin-3-yl)amino)pyrimidin-2-yl)-3-fluoropiperidin-4-yl)oxy)piperidin-1-yl)-2-(2,6-dioxopiperidin-3-yl)isoindoline-1,3-dione,
1-((R)-2-(3-((2-((3S,4R)-3-fluoro-4-morpholinopiperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3'S,4′R)-3′-fluoro-4-hydroxy-4-methyl-[1,4′-bipiperidin]-1′-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
4R)-3-fluoro-4-(4-(tetrahydro-2H-pyran-4-yl)piperazin-1-yl)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-(4-(6-(trifluoromethyl)pyridin-2-yl)piperazin-1-yl)piperidin-1-yl)pyrimidin-4-yl)amino)-5-isopropyl-8-(3-((methylsulfonyl)methyl)azetidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-((S)-3-((methylsulfonyl)methyl)pyrrolidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-((R)-3-((methylsulfonyl)methyl)pyrrolidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-(4-((methylsulfonyl)methyl)piperidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-((R)-3-((methylsulfonyl)methyl)piperidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-((S)-3-((methylsulfonyl)methyl)piperidin-1-yl)isoquinolin-6-yl)pyrrolidin-1-yl)prop-2-en-1-one,
1-((R)-2-(8-(3-((S)-1,1-dioxidotetrahydrothiophen-2-yl)azetidin-1-yl)-3-((2-((3S,4R)-3-fluoro-4-methoxypiperidin-1-yl)pyrimidin-4-yl)amino)-5-isopropylisoquinolin-6-yl)pyrrolidin-1-yl)prop-2-en-1-one,
1-((R)-2-(8-(3-((R)-1,1-dioxidotetrahydrothiophen-2-yl)azetidin-1-yl)-3-((2-((3S,4R)-3-fluoro-4-methoxypiperidin-1-yl)pyrimidin-4-yl)amino)-5-isopropylisoquinolin-6-yl)pyrrolidin-1-yl)prop-2-en-1-one,
1-((R)-2-(8-(3-((R)-1,1-dioxidotetrahydro-2H-thiopyran-2-yl)azetidin-1-yl)-3-((2-((3S,4R)-3-fluoro-4-methoxypiperidin-1-yl)pyrimidin-4-yl)amino)-5-isopropylisoquinolin-6-yl)pyrrolidin-1-yl)prop-2-en-1-one,
1-((R)-2-(8-(3-((S)-1,1-dioxidotetrahydro-2H-thiopyran-2-yl)azetidin-1-yl)-3-((2-((3S,4R)-3-fluoro-4-methoxypiperidin-1-yl)pyrimidin-4-yl)amino)-5-isopropylisoquinolin-6-yl)pyrrolidin-1-yl)prop-2-en-1-one,
1-((R)-2-(8-(3-((S)-4,4-dioxido-1,4-oxathian-3-yl)azetidin-1-yl)-3-((2-((3S,4R)-3-fluoro-4-methoxypiperidin-1-yl)pyrimidin-4-yl)amino)-5-isopropylisoquinolin-6-yl)pyrrolidin-1-yl)prop-2-en-1-one,
1-((R)-2-(8-(3-((R)-4,4-dioxido-1,4-oxathian-3-yl)azetidin-1-yl)-3-((2-((3S,4R)-3-fluoro-4-methoxypiperidin-1-yl)pyrimidin-4-yl)amino)-5-isopropylisoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-8-(3-((2R,4S)-4-hydroxy-4-methyl-1,1-dioxidotetrahydro-2H-thiopyran-2-yl)azetidin-1-yl)-5-isopropylisoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-8-(3-((2R,4R)-4-hydroxy-4-methyl-1,1-dioxidotetrahydro-2H-thiopyran-2-yl)azetidin-1-yl)-5-isopropylisoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-8-(3-((2S,4S)-4-hydroxy-4-methyl-1,1-dioxidotetrahydro-2H-thiopyran-2-yl)azetidin-1-yl)-5-isopropylisoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-8-(3-((2S,4R)-4-hydroxy-4-methyl-1,1-dioxidotetrahydro-2H-thiopyran-2-yl)azetidin-1-yl)-5-isopropylisoquinolin-6-yl)pyrrolidin-1-yl)prop-2-en-1-one,
1-((R)-2-(8-(3-((S)-1,1-dioxido-4-(tetrahydro-2H-pyran-4-yl)thiomorpholin-2-yl)azetidin-1-yl)-3-((2-((3S,4R)-3-fluoro-4-methoxypiperidin-1-yl)pyrimidin-4-yl)amino)-5-isopropyl isoquinolin-6-yl)pyrrolidin-1-yl)prop-2-en-1-one,
1-((R)-2-(8-(3-((R)-1,1-dioxido-4-(tetrahydro-2H-pyran-4-yl)thiomorpholin-2-yl)azetidin-1-yl)-3-((2-((3S,4R)-3-fluoro-4-methoxypiperidin-1-yl)pyrimidin-4-yl)amino)-5-isopropyl isoquinolin-6-yl)pyrrolidin-1-yl)prop-2-en-1-one,
1-((R)-2-(8-(3-((R)-1,1-dioxido-4-(2-(trifluoromethyl)pyridin-4-yl)thiomorpholin-2-yl)azetidin-1-yl)-3-((2-((3S,4R)-3-fluoro-4-methoxypiperidin-1-yl)pyrimidin-4-yl)amino)-5-isopropyl isoquinolin-6-yl)pyrrolidin-1-yl)prop-2-en-1-one,
1-((R)-2-(8-(3-((S)-1,1-dioxido-4-(2-(trifluoromethyl)pyridin-4-yl)thiomorpholin-2-yl)azetidin-1-yl)-3-((2-((3S,4R)-3-fluoro-4-methoxypiperidin-1-yl)pyrimidin-4-yl)amino)-5-isopropyl isoquinolin-6-yl)pyrrolidin-1-yl)prop-2-en-1-one,
5-((R)-2-(1-(6-((R)-1-acryloylpyrrolidin-2-yl)-3-((2-((3S,4R)-3-fluoro-4-methoxypiperidin-1-yl)pyrimidin-4-yl)amino)-5-isopropylisoquinolin-8-yl)azetidin-3-yl)-1,1-dioxidothiomorpholino)-2-(2,6-dioxopiperidin-3-yl)isoindoline-1,3-dione,
5-((S)-2-(1-(6-((R)-1-acryloylpyrrolidin-2-yl)-3-((2-((3S,4R)-3-fluoro-4-methoxypiperidin-1-yl)pyrimidin-4-yl)amino)-5-isopropylisoquinolin-8-yl)azetidin-3-yl)-1,1-dioxidothiomorpholino)-2-(2,6-dioxopiperidin-3-yl)isoindoline-1,3-dione,
1-((R)-2-(3-((2-((3S,4R)-3-fluoro-4-methoxypiperidin-1-yl)pyrimidin-4-yl)amino)-5-isopropyl-8-(3-((2R,6S)-6-methyl-1,1-dioxidotetrahydro-2H-thiopyran-2-yl)azetidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-(3-((2R,6R)-6-methyl-1,1-dioxidotetrahydro-2H-thiopyran-2-yl)azetidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-(3-((2S,6S)-6-methyl-1,1-dioxidotetrahydro-2H-thiopyran-2-yl)azetidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-(3-((2S,6R)-6-methyl-1,1-dioxidotetrahydro-2H-thiopyran-2-yl)azetidin-1-yl)isoquinolin-6-yl)pyrrolidin-1-yl)prop-2-en-1-one,
1-((R)-2-(8-(3-((R)-6,6-dimethyl-1,1-dioxidotetrahydro-2H-thiopyran-2-yl)azetidin-1-yl)-3-((2-((3S,4R)-3-fluoro-4-methoxypiperidin-1-yl)pyrimidin-4-yl)amino)-5-isopropylisoquinolin-6-yl)pyrrolidin-1-yl)prop-2-en-1-one,
1-((R)-2-(8-(3-((S)-6,6-dimethyl-1,1-dioxidotetrahydro-2H-thiopyran-2-yl)azetidin-1-yl)-3-((2-((3S,4R)-3-fluoro-4-methoxypiperidin-1-yl)pyrimidin-4-yl)amino)-5-isopropylisoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-(3-(2-(isopropylsulfonyl)propan-2-yl)azetidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-(3-(1-(isopropylsulfonyl)cyclobutyl)azetidin-1-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-((1 S,2S,6R)-2-(methylsulfonyl)-7-azabicyclo[4.2.0]octan-7-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-((1 S,2R,6R)-2-(methylsulfonyl)-7-azabicyclo[4.2.0]octan-7-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-((S)-5-(methylsulfonyl)-2-azaspiro[3.4]octan-2-yl)isoquinolin-6-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-5-isopropyl-8-((R)-5-(methylsulfonyl)-2-azaspiro[3.4]octan-2-yl)isoquinolin-6-yl)pyrrolidin-1-yl)prop-2-en-1-one,
2-fluoro-N-(3-((2-((3S,4R)-3-fluoro-4-methoxypiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((2S,4R,5S)-5-fluoro-4-methoxy-2-methylpiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((2R,4R,5S)-5-fluoro-4-methoxy-2-methylpiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-isopropoxypiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-4-(tert-butoxy)-3-fluoropiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-(trifluoromethoxy)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-(2-hydroxyethoxy)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-(2-methoxyethoxy)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-(2-(methylamino)ethoxy)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-(3-morpholinopropoxy)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-((tetrahydro-2H-pyran-4-yl)oxy)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-((4-hydroxy-4-methylcyclohexyl)oxy)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-((1-(tetrahydro-2H-pyran-4-yl)piperidin-4-yl)oxy)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-((1-(6-(trifluoromethyl)pyridin-2-yl)piperidin-4-yl)oxy)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-4-((1-(2-(2,6-dioxopiperidin-3-yl)-1,3-dioxoisoindolin-5-yl)piperidin-4-yl)oxy)-3-fluoropiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-morpholinopiperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3'S,4′R)-3′-fluoro-4-hydroxy-4-methyl-[1,4′-bipiperidin]-1′-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-(4-(tetrahydro-2H-pyran-4-yl)piperazin-1-yl)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(3-((2-((3S,4R)-3-fluoro-4-(4-(6-(trifluoromethyl)pyridin-2-yl)piperazin-1-yl)piperidin-1-yl)pyrimidin-4-yl)amino)-5-isopropyl-8-((2R,3S)-2-methyl-3-((methylsulfonyl)methyl)azetidin-1-yl)isoquinolin-6-yl)acrylamide,
N-(8-(3-(4,4-dioxido-1,4-oxathian-3-yl)-2-methylazetidin-1-yl)-3-((2-((3S,4R)-3-fluoro-4-methoxypiperidin-1-yl)pyrimidin-4-yl)amino)-5-isopropylisoquinolin-6-yl)acrylamide,
N-(3-((2-((3S,4R)-3-fluoro-4-methoxypiperidin-1-yl)pyrimidin-4-yl)amino)-8-(3-(4-hydroxy-4-methyl-1,1-dioxidotetrahydro-2H-thiopyran-2-yl)-2-methylazetidin-1-yl)-5-isopropylisoquinolin-6-yl)acrylamide,
N-(8-(3-(1,1-dioxido-4-(tetrahydro-2H-pyran-4-yl)thiomorpholin-2-yl)-2-methylazetidin-1-yl)-3-((2-((3S,4R)-3-fluoro-4-methoxypiperidin-1-yl)pyrimidin-4-yl)amino)-5-isopropylisoquinolin-6-yl)acrylamide,
N-(8-(3-(1,1-dioxido-4-(2-(trifluoromethyl)pyridin-4-yl)thiomorpholin-2-yl)-2-methylazetidin-1-yl)-3-((2-((3S,4R)-3-fluoro-4-methoxypiperidin-1-yl)pyrimidin-4-yl)amino)-5-isopropylisoquinolin-6-yl)acrylamide,
N-(8-(3-(4-(2-(2,6-dioxopiperidin-3-yl)-1,3-dioxoisoindolin-5-yl)-1,1-dioxidothiomorpholin-2-yl)-2-methylazetidin-1-yl)-3-((2-((3S,4R)-3-fluoro-4-methoxypiperidin-1-yl)pyrimidin-4-yl)amino)-5-isopropylisoquinolin-6-yl)acrylamide,
N-(3-((2-((3S,4R)-3-fluoro-4-methoxypiperidin-1-yl)pyrimidin-4-yl)amino)-5-isopropyl-8-(2-methyl-3-(6-methyl-1,1-dioxidotetrahydro-2H-thiopyran-2-yl)azetidin-1-yl)isoquinolin-6-yl)acrylamide,
N-(8-(3-(6,6-dimethyl-1,1-dioxidotetrahydro-2H-thiopyran-2-yl)-2-methyl azetidin-1-yl)-3-((2-((3S,4R)-3-fluoro-4-methoxypiperidin-1-yl)pyrimidin-4-yl)amino)-5-isopropylisoquinolin-6-yl)acrylamide,
N-(8-(3-(4,4-dioxido-1,4-oxathian-3-yl)azetidin-1-yl)-3-((2-((3S,4R)-3-fluoro-4-methoxypiperidin-1-yl)pyrimidin-4-yl)amino)-5-isopropylisoquinolin-6-yl)acrylamide,
N-(3-((2-((3S,4R)-3-fluoro-4-methoxypiperidin-1-yl)pyrimidin-4-yl)amino)-8-(3-(4-hydroxy-4-methyl-1,1-dioxidotetrahydro-2H-thiopyran-2-yl)azetidin-1-yl)-5-isopropylisoquinolin-6-yl)acrylamide,
N-(8-(3-(1,1-dioxido-4-(tetrahydro-2H-pyran-4-yl)thiomorpholin-2-yl)azetidin-1-yl)-3-((2-((3S,4R)-3-fluoro-4-methoxypiperidin-1-yl)pyrimidin-4-yl)amino)-5-isopropylisoquinolin-6-yl)acrylamide,
N-(8-(3-(1,1-dioxido-4-(2-(trifluoromethyl)pyridin-4-yl)thiomorpholin-2-yl)azetidin-1-yl)-3-((2-((3S,4R)-3-fluoro-4-methoxypiperidin-1-yl)pyrimidin-4-yl)amino)-5-isopropylisoquinolin-6-yl)acrylamide,
N-(3-((2-((3S,4R)-3-fluoro-4-methoxypiperidin-1-yl)pyrimidin-4-yl)amino)-5-isopropyl-8-(3-(6-methyl-1,1-dioxidotetrahydro-2H-thiopyran-2-yl)azetidin-1-yl)isoquinolin-6-yl)acrylamide,
N-(8-(3-(6,6-dimethyl-1,1-dioxidotetrahydro-2H-thiopyran-2-yl)azetidin-1-yl)-3-((2-((3S,4R)-3-fluoro-4-methoxypiperidin-1-yl)pyrimidin-4-yl)amino)-5-isopropylisoquinolin-6-yl)acrylamide,
N-(8-(3-(4-(2-(2,6-dioxopiperidin-3-yl)-1,3-dioxoisoindolin-5-yl)-1,1-dioxidothiomorpholin-2-yl)azetidin-1-yl)-3-((2-((3S,4R)-3-fluoro-4-methoxypiperidin-1-yl)pyrimidin-4-yl)amino)-5-isopropylisoquinolin-6-yl)acrylamide,
2-fluoro-N-(2-((2-((3S,4R)-3-fluoro-4-methoxypiperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((2S,4R,5S)-5-fluoro-4-methoxy-2-methylpiperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((2R,4R,5S)-5-fluoro-4-methoxy-2-methylpiperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-isopropoxypiperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-4-(tert-butoxy)-3-fluoropiperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-(trifluoromethoxy)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-(2-hydroxyethoxy)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-(2-methoxyethoxy)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-(2-(methylamino)ethoxy)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-(3-morpholinopropoxy)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-((tetrahydro-2H-pyran-4-yl)oxy)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-((4-hydroxy-4-methylcyclohexyl)oxy)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-((1-(tetrahydro-2H-pyran-4-yl)piperidin-4-yl)oxy)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-((1-(6-(trifluoromethyl)pyridin-2-yl)piperidin-4-yl)oxy)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-4-((1-(2-(2,6-dioxopiperidin-3-yl)-1,3-dioxoisoindolin-5-yl)piperidin-4-yl)oxy)-3-fluoropiperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-morpholinopiperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3'S,4′R)-3′-fluoro-4-hydroxy-4-methyl-[1,4′-bipiperidin]-1′-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-(4-(tetrahydro-2H-pyran-4-yl)piperazin-1-yl)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(2-((2-((3S,4R)-3-fluoro-4-(4-(6-(trifluoromethyl)pyridin-2-yl)piperazin-1-yl)piperidin-1-yl)pyridin-4-yl)amino)-8-isopropyl-5-((2R,3S)-2-methyl-3-((methylsulfonyl)methyl)azetidin-1-yl)quinazolin-7-yl)acrylamide,
N-(5-(3-(4,4-dioxido-1,4-oxathian-3-yl)-2-methylazetidin-1-yl)-2-((2-((3S,4R)-3-fluoro-4-methoxypiperidin-1-yl)pyridin-4-yl)amino)-8-isopropylquinazolin-7-yl)acrylamide,
N-(2-((2-((3S,4R)-3-fluoro-4-methoxypiperidin-1-yl)pyridin-4-yl)amino)-5-(3-(4-hydroxy-4-methyl-1,1-dioxidotetrahydro-2H-thiopyran-2-yl)-2-methylazetidin-1-yl)-8-isopropylquinazolin-7-yl)acrylamide,
N-(5-(3-(1,1-dioxido-4-(tetrahydro-2H-pyran-4-yl)thiomorpholin-2-yl)-2-methylazetidin-1-yl)-2-((2-((3S,4R)-3-fluoro-4-methoxypiperidin-1-yl)pyridin-4-yl)amino)-8-isopropylquinazolin-7-yl)acrylamide,
N-(5-(3-(1,1-dioxido-4-(2-(trifluoromethyl)pyridin-4-yl)thiomorpholin-2-yl)-2-methylazetidin-1-yl)-2-((2-((3S,4R)-3-fluoro-4-methoxypiperidin-1-yl)pyridin-4-yl)amino)-8-isopropylquinazolin-7-yl)acrylamide,
N-(5-(3-(4-(2-(2,6-dioxopiperidin-3-yl)-1,3-dioxoisoindolin-5-yl)-1,1-dioxidothiomorpholin-2-yl)-2-methylazetidin-1-yl)-2-((2-((3S,4R)-3-fluoro-4-methoxypiperidin-1-yl)pyridin-4-yl)amino)-8-isopropylquinazolin-7-yl)acrylamide,
N-(2-((2-((3S,4R)-3-fluoro-4-methoxypiperidin-1-yl)pyridin-4-yl)amino)-8-isopropyl-5-(2-methyl-3-(6-methyl-1,1-dioxidotetrahydro-2H-thiopyran-2-yl)azetidin-1-yl)quinazolin-7-yl)acrylamide,
N-(5-(3-(6,6-dimethyl-1,1-dioxidotetrahydro-2H-thiopyran-2-yl)-2-methyl azetidin-1-yl)-2-((2-((3S,4R)-3-fluoro-4-methoxypiperidin-1-yl)pyridin-4-yl)amino)-8-isopropylquinazolin-7-yl)acrylamide,
N-(5-(3-(4,4-dioxido-1,4-oxathian-3-yl)azetidin-1-yl)-2-((2-((3S,4R)-3-fluoro-4-methoxypiperidin-1-yl)pyridin-4-yl)amino)-8-isopropylquinazolin-7-yl)acrylamide,
N-(2-((2-((3S,4R)-3-fluoro-4-methoxypiperidin-1-yl)pyridin-4-yl)amino)-5-(3-(4-hydroxy-4-methyl-1,1-dioxidotetrahydro-2H-thiopyran-2-yl)azetidin-1-yl)-8-isopropylquinazolin-7-yl)acrylamide,
N-(5-(3-(1,1-dioxido-4-(tetrahydro-2H-pyran-4-yl)thiomorpholin-2-yl)azetidin-1-yl)-2-((2-((3S,4R)-3-fluoro-4-methoxypiperidin-1-yl)pyridin-4-yl)amino)-8-isopropylquinazolin-7-yl)acrylamide,
N-(5-(3-(1,1-dioxido-4-(2-(trifluoromethyl)pyridin-4-yl)thiomorpholin-2-yl)azetidin-1-yl)-2-((2-((3S,4R)-3-fluoro-4-methoxypiperidin-1-yl)pyridin-4-yl)amino)-8-isopropylquinazolin-7-yl)acrylamide,
N-(2-((2-((3S,4R)-3-fluoro-4-methoxypiperidin-1-yl)pyridin-4-yl)amino)-8-isopropyl-5-(3-(6-methyl-1,1-dioxidotetrahydro-2H-thiopyran-2-yl)azetidin-1-yl)quinazolin-7-yl)acrylamide,
N-(5-(3-(6,6-dimethyl-1,1-dioxidotetrahydro-2H-thiopyran-2-yl)azetidin-1-yl)-2-((2-((3S,4R)-3-fluoro-4-methoxypiperidin-1-yl)pyridin-4-yl)amino)-8-isopropylquinazolin-7-yl)acrylamide,
N-(5-(3-(4-(2-(2,6-dioxopiperidin-3-yl)-1,3-dioxoisoindolin-5-yl)-1,1-dioxidothiomorpholin-2-yl)azetidin-1-yl)-2-((2-((3S,4R)-3-fluoro-4-methoxypiperidin-1-yl)pyridin-4-yl)amino)-8-isopropylquinazolin-7-yl)acrylamide,
N-(2-(3-((2-((3S,4R)-3-fluoro-4-hydroxy-3-methylpiperidin-1-yl)pyrimidin-4-yl)amino)-8-((2R,3S)-2-methyl-3-((methylsulfonyl)methyl)azetidin-1-yl)isoquinolin-5-yl)propan-2-yl)acrylamide,
(E)-4-(dimethylamino)-N-(2-(3-((2-((3S,4R)-3-fluoro-4-hydroxy-3-methylpiperidin-1-yl)pyrimidin-4-yl)amino)-8-((2R,3S)-2-methyl-3-((methylsulfonyl)methyl)azetidin-1-yl)isoquinolin-5-yl)propan-2-yl)but-2-enamide,
N-(2-(3-((2-((3S,4R)-3-fluoro-4-hydroxy-3-methylpiperidin-1-yl)pyrimidin-4-yl)amino)-8-((2R,3S)-2-methyl-3-((methylsulfonyl)methyl)azetidin-1-yl)isoquinolin-5-yl)propan-2-yl)but-2-ynamide,
(E)-N-(2-(3-((2-((3S,4R)-3-fluoro-4-hydroxy-3-methylpiperidin-1-yl)pyrimidin-4-yl)amino)-8-((2R,3S)-2-methyl-3-((methylsulfonyl)methyl)azetidin-1-yl)isoquinolin-5-yl)propan-2-yl)-4-methyl-4-(4-(oxetan-3-yl)piperazin-1-yl)pent-2-enamide,
N-(2-(3-((2-(4-methoxypiperidin-1-yl)pyrimidin-4-yl)amino)-8-((2R, 3S)-2-methyl-3-((methylsulfonyl)methyl)azetidin-1-yl)isoquinolin-5-yl)propyl)acrylamide,
N-(2-(3-((2-(4-methoxypiperidin-1-yl)pyrimidin-4-yl)amino)-8-((2R, 3S)-2-methyl-3-((methylsulfonyl)methyl)azetidin-1-yl)isoquinolin-5-yl)-2-methylpropyl)acrylamide,
N-(1-(3-((2-(4-methoxypiperidin-1-yl)pyrimidin-4-yl)amino)-8-((2R, 3S)-2-methyl-3-((methylsulfonyl)methyl)azetidin-1-yl)isoquinolin-5-yl)ethyl)acrylamide,
N-(2-(3-((2-(4-methoxypiperidin-1-yl)pyrimidin-4-yl)amino)-8-((2R, 3S)-2-methyl-3-((methylsulfonyl)methyl)azetidin-1-yl)isoquinolin-5-yl)propan-2-yl)acrylamide,
1-(3-(3-((2-(4-methoxypiperidin-1-yl)pyrimidin-4-yl)amino)-8-((2R, 3S)-2-methyl-3-((methylsulfonyl)methyl)azetidin-1-yl)isoquinolin-5-yl)azetidin-1-yl)prop-2-en-1-one,
1-((S)-2-(3-((2-(4-methoxypiperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((R)-2-(3-((2-(4-methoxypiperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-(4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-(4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-(4-methoxypiperidin-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-(4-methoxypiperidin-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-(4-methoxypiperidin-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
1-((R)-2-(3-((2-(4-methoxypiperidin-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
N-(2-(3-((2-(4-hydroxy-4-methylpi peridin-1-yl)pyrimidin-4-yl)amino)-8-((2R,3S)-2-methyl-3-((methylsulfonyl)methyl)azetidin-1-yl)isoquinolin-5-yl)propan-2-yl)acrylamide,
1-(3-(3-((2-(4-hydroxy-4-methyl piperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
N-(2-(3-((2-(4-((3aS,6aR)-hexahydro-1H-furo[3,4-b]pyrrol-1-yl)piperidin-1-yl)pyrimidin-4-yl)amino)-8-((2R,3S)-2-methyl-3-((methylsulfonyl)methyl)azetidin-1-yl)isoquinolin-5-yl)propan-2-yl)acrylamide,
1-(3-(3-((2-((3aS,6aR)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((3aS,6aR)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((R)-2-(3-((2-((3aS,6aR)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((3aS,6aR)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((3aS,6aR)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((3aS,6aR)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((3aS,6aR)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((3aS,6aR)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
1-((R)-2-(3-((2-((3aS,6aR)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
N-(2-(3-((2-(4-((3aR,6aS)-hexahydro-1H-furo[3,4-b]pyrrol-1-yl)piperidin-1-yl)pyrimidin-4-yl)amino)-8-((2R,3S)-2-methyl-3-((methylsulfonyl)methyl)azetidin-1-yl)isoquinolin-5-yl)propan-2-yl)acrylamide,
1-(3-(3-((2-((3aR,6aS)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((3aR,6aS)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((R)-2-(3-((2-((3aR,6aS)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((3aR,6aS)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((3aR,6aS)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((3aR,6aS)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((3aR,6aS)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((3aR,6aS)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
1-((R)-2-(3-((2-((3aR,6aS)-hexahydro-1H-furo[3,4-b]pyrrol-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
N-(2-(3-((2-(4-((1 S,5R)-6-oxa-2-azabicyclo[3.2.1]octan-2-yl)piperidin-1-yl)pyrimidin-4-yl)amino)-8-((2R,3S)-2-methyl-3-((methylsulfonyl)methyl)azetidin-1-yl)isoquinolin-5-yl)propan-2-yl)acrylamide,
1-(3-(3-((2-((1 S,5R)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((1 S,5R)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((R)-2-(3-((2-((1 S,5R)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((1 S,5R)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((1 S,5R)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((1 S,5R)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((1 S,5R)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((1 S,5R)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
1-((R)-2-(3-((2-((1 S,5R)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
N-(2-(3-((2-(4-((1R,5S)-6-oxa-2-azabicyclo[3.2.1]octan-2-yl)piperidin-1-yl)pyrimidin-4-yl)amino)-8-((2R,3S)-2-methyl-3-((methylsulfonyl)methyl)azetidin-1-yl)isoquinolin-5-yl)propan-2-yl)acrylamide,
1-(3-(3-((2-((1R,5S)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((1R,5S)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((R)-2-(3-((2-((1R,5S)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((1R,5S)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((1R,5S)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((1R,5S)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((1R,5S)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((1R,5S)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
1-((R)-2-(3-((2-((1R,5S)-6-oxa-2-azabicyclo[3.2.1]octan-2-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
N-(2-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)propan-2-yl)acrylamide,
1-(3-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((R)-2-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
1-((R)-2-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
N-(2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)propan-2-yl)acrylamide,
1-(3-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((R)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)azetidin-1-yl)prop-2-en-1-one,
1-((S)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)piperidin-1-yl)prop-2-en-1-one,
1-((S)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
1-((R)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)azepan-1-yl)prop-2-en-1-one,
N-(2-(3-((2-(4-hydroxy-4-methylpi peridin-1-yl)pyrimidin-4-yl)amino)-8-((2R,3S)-2-methyl-3-(((trifluoromethyl)sulfonyl)methyl)azetidin-1-yl)isoquinolin-5-yl)propan-2-yl)acrylamide,
1-(3-(3-((2-(4-hydroxy-4-methyl piperidin-1-yl)pyrimidin-4-yl)amino)-8-((2R,3S)-2-methyl-3-(((trifluoromethyl)sulfonyl)methyl)azetidin-1-yl)isoquinolin-5-yl)azetidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-(((trifluoromethyl)sulfonyl)methyl)azetidin-1-yl)isoquinolin-5-yl)azetidin-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-(((trifluoromethyl)sulfonyl)methyl)azetidin-1-yl)isoquinolin-5-yl)azetidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-(((trifluoromethyl)sulfonyl)methyl)azetidin-1-yl)isoquinolin-5-yl)pyrrolidin-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-(((trifluoromethyl)sulfonyl)methyl)azetidin-1-yl)isoquinolin-5-yl)pyrrolidin-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-(((trifluoromethyl)sulfonyl)methyl)azetidin-1-yl)isoquinolin-5-yl)piperidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-(((trifluoromethyl)sulfonyl)methyl)azetidin-1-yl)isoquinolin-5-yl)piperidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-(((trifluoromethyl)sulfonyl)methyl)azetidin-1-yl)isoquinolin-5-yl)azepan-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-(((trifluoromethyl)sulfonyl)methyl)azetidin-1-yl)isoquinolin-5-yl)azepan-1-yl)prop-2-en-1-one,
N-(2-(3-((2-(4-hydroxy-4-methylpi peridin-1-yl)pyrimidin-4-yl)amino)-8-((2R,3S)-2-methyl-3-(2-(methylsulfonyl)propan-2-yl)azetidin-1-yl)isoquinolin-5-yl)propan-2-yl)acrylamide,
1-(3-(3-((2-(4-hydroxy-4-methyl piperidin-1-yl)pyrimidin-4-yl)amino)-8-((2R,3S)-2-methyl-3-(2-(methylsulfonyl)propan-2-yl)azetidin-1-yl)isoquinolin-5-yl)azetidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-(2-(methylsulfonyl)propan-2-yl)azetidin-1-yl)isoquinolin-5-yl)azetidin-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-(2-(methylsulfonyl)propan-2-yl)azetidin-1-yl)isoquinolin-5-yl)azetidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-(2-(methylsulfonyl)propan-2-yl)azetidin-1-yl)isoquinolin-5-yl)pyrrolidin-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-(2-(methylsulfonyl)propan-2-yl)azetidin-1-yl)isoquinolin-5-yl)pyrrolidin-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-(2-(methylsulfonyl)propan-2-yl)azetidin-1-yl)isoquinolin-5-yl)piperidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-(2-(methylsulfonyl)propan-2-yl)azetidin-1-yl)isoquinolin-5-yl)piperidin-1-yl)prop-2-en-1-one,
1-((S)-2-(3-((2-(4-hydroxy-4-methylpiperidin-1-yl)pyrimidin-4-yl)amino)-8-((2R,3S)-2-methyl-3-(2-(methylsulfonyl)propan-2-yl)azetidin-1-yl)isoquinolin-5-yl)azepan-1-yl)prop-2-en-1-one,
1-((R)-2-(3-((2-(4-hydroxy-4-methylpiperidin-1-yl)pyrimidin-4-yl)amino)-8-((2R,3S)-2-methyl-3-(2-(methylsulfonyl)propan-2-yl)azetidin-1-yl)isoquinolin-5-yl)azepan-1-yl)prop-2-en-1-one,
N-(2-(3-((2-(4-hydroxy-4-methylpiperidin-1-yl)pyrimidin-4-yl)amino)-8-((3R,4R)-2,2,4-trimethyl-3-((methylsulfonyl)methyl)azetidin-1-yl)isoquinolin-5-yl)propan-2-yl)acrylamide,
1-(3-(3-((2-(4-hydroxy-4-methyl piperidin-1-yl)pyrimidin-4-yl)amino)-8-((3R,4R)-2,2,4-trimethyl-3-((methylsulfonyl)methyl)azetidin-1-yl)isoquinolin-5-yl)azetidin-1-yl)prop-2-en-1-one,
1-((S)-2-(3-((2-(4-hydroxy-4-methylpiperidin-1-yl)pyrimidin-4-yl)amino)-8-((3R,4R)-2,2,4-trimethyl-3-((methylsulfonyl)methyl)azetidin-1-yl)isoquinolin-5-yl)azetidin-1-yl)prop-2-en-1-one,
1-((R)-2-(3-((2-(4-hydroxy-4-methylpiperidin-1-yl)pyrimidin-4-yl)amino)-8-((3R,4R)-2,2,4-trimethyl-3-((methylsulfonyl)methyl)azetidin-1-yl)isoquinolin-5-yl)azetidin-1-yl)prop-2-en-1-one,
1-((S)-2-(3-((2-(4-hydroxy-4-methylpiperidin-1-yl)pyrimidin-4-yl)amino)-8-((3R,4R)-2,2,4-trimethyl-3-((methylsulfonyl)methyl)azetidin-1-yl)isoquinolin-5-yl)pyrrolidin-1-yl)prop-2-en-1-one,
1-((R)-2-(3-((2-(4-hydroxy-4-methylpiperidin-1-yl)pyrimidin-4-yl)amino)-8-((3R,4R)-2,2,4-trimethyl-3-((methylsulfonyl)methyl)azetidin-1-yl)isoquinolin-5-yl)pyrrolidin-1-yl)prop-2-en-1-one,
1-((R)-2-(3-((2-(4-hydroxy-4-methylpiperidin-1-yl)pyrimidin-4-yl)amino)-8-((3R,4R)-2,2,4-trimethyl-3-((methylsulfonyl)methyl)azetidin-1-yl)isoquinolin-5-yl)piperidin-1-yl)prop-2-en-1-one,
1-((S)-2-(3-((2-(4-hydroxy-4-methylpiperidin-1-yl)pyrimidin-4-yl)amino)-8-((3R,4R)-2,2,4-trimethyl-3-((methylsulfonyl)methyl)azetidin-1-yl)isoquinolin-5-yl)piperidin-1-yl)prop-2-en-1-one,
1-((S)-2-(3-((2-(4-hydroxy-4-methylpiperidin-1-yl)pyrimidin-4-yl)amino)-8-((3R,4R)-2,2,4-trimethyl-3-((methylsulfonyl)methyl)azetidin-1-yl)isoquinolin-5-yl)azepan-1-yl)prop-2-en-1-one,
1-((R)-2-(3-((2-(4-hydroxy-4-methylpiperidin-1-yl)pyrimidin-4-yl)amino)-8-((3R,4R)-2,2,4-trimethyl-3-((methylsulfonyl)methyl)azetidin-1-yl)isoquinolin-5-yl)azepan-1-yl)prop-2-en-1-one,
(S)—N-(2-(8-(2,2-dimethyl-3-((methylsulfonyl)methyl)azetidin-1-yl)-3-((2-(4-hydroxy-4-methylpiperidin-1-yl)pyrimidin-4-yl)amino)isoquinolin-5-yl)propan-2-yl)acrylamide,
(S)-1-(3-(8-(2,2-dimethyl-3-((methylsulfonyl)methyl)azetidin-1-yl)-3-((2-(4-hydroxy-4-methyl pi peridin-1-yl)pyrimidin-4-yl)amino)isoquinolin-5-yl)azetidin-1-yl)prop-2-en-1-one,
1-((S)-2-(8-((S)-2,2-dimethyl-3-((methylsulfonyl)methyl)azetidin-1-yl)-3-((2-(4-hydroxy-4-methylpiperidin-1-yl)pyrimidin-4-yl)amino)isoquinolin-5-yl)azetidin-1-yl)prop-2-en-1-one,
1-((R)-2-(8-((S)-2,2-dimethyl-3-((methylsulfonyl)methyl)azetidin-1-yl)-3-((2-(4-hydroxy-4-methylpiperidin-1-yl)pyrimidin-4-yl)amino)isoquinolin-5-yl)azetidin-1-yl)prop-2-en-1-one,
1-((S)-2-(8-((S)-2,2-dimethyl-3-((methylsulfonyl)methyl)azetidin-1-yl)-3-((2-(4-hydroxy-4-methylpiperidin-1-yl)pyrimidin-4-yl)amino)isoquinolin-5-yl)pyrrolidin-1-yl)prop-2-en-1-one,
1-((R)-2-(8-((S)-2,2-dimethyl-3-((methylsulfonyl)methyl)azetidin-1-yl)-3-((2-(4-hydroxy-4-methylpiperidin-1-yl)pyrimidin-4-yl)amino)isoquinolin-5-yl)pyrrolidin-1-yl)prop-2-en-1-one,
1-((R)-2-(8-((S)-2,2-dimethyl-3-((methylsulfonyl)methyl)azetidin-1-yl)-3-((2-(4-hydroxy-4-methylpiperidin-1-yl)pyrimidin-4-yl)amino)isoquinolin-5-yl)piperidin-1-yl)prop-2-en-1-one,
1-((S)-2-(8-((S)-2,2-dimethyl-3-((methylsulfonyl)methyl)azetidin-1-yl)-3-((2-(4-hydroxy-4-methylpiperidin-1-yl)pyrimidin-4-yl)amino)isoquinolin-5-yl)piperidin-1-yl)prop-2-en-1-one,
1-((S)-2-(8-((S)-2,2-dimethyl-3-((methylsulfonyl)methyl)azetidin-1-yl)-3-((2-(4-hydroxy-4-methylpiperidin-1-yl)pyrimidin-4-yl)amino)isoquinolin-5-yl)azepan-1-yl)prop-2-en-1-one,
1-((R)-2-(8-((S)-2,2-dimethyl-3-((methylsulfonyl)methyl)azetidin-1-yl)-3-((2-(4-hydroxy-4-methylpiperidin-1-yl)pyrimidin-4-yl)amino)isoquinolin-5-yl)azepan-1-yl)prop-2-en-1-one,
N-(2-(3-((2-(4-hydroxy-4-methylpi peridin-1-yl)pyrimidin-4-yl)amino)-8-((2R,3S)-3-(N-isopropylmethylsulfonamido)-2-methylazetidin-1-yl)isoquinolin-5-yl)propan-2-yl)acrylamide,
N-((2R, 3S)-1-(5-(1-acryloylazetidin-3-yl)-3-((2-(4-hydroxy-4-methyl piperidin-1-yl)pyrimidin-4-yl)amino)isoquinolin-8-yl)-2-methylazetidin-3-yl)-N-isopropylmethanesuIfonamide,
N-((2R, 3S)-1-(5-((S)-1-acryloylazetidin-2-yl)-3-((2-(4-hydroxy-4-methylpi peridin-1-yl)pyrimidin-4-yl)amino)isoquinolin-8-yl)-2-methylazetidin-3-yl)-N-isopropylmethanesuIfonamide,
N-((2R, 3S)-1-(5-((R)-1-acryloylazetidin-2-yl)-3-((2-(4-hydroxy-4-methylpi peridin-1-yl)pyrimidin-4-yl)amino)isoquinolin-8-yl)-2-methylazetidin-3-yl)-N-isopropylmethanesuIfonamide,
N-((2R, 3S)-1-(5-((S)-1-acryloylpyrrolidin-2-yl)-3-((2-(4-hydroxy-4-methylpiperidin-1-yl)pyrimidin-4-yl)amino)isoquinolin-8-yl)-2-methylazetidin-3-yl)-N-isopropylmethanesuIfonamide,
N-((2R, 3S)-1-(5-((R)-1-acryloylpyrrolidin-2-yl)-3-((2-(4-hydroxy-4-methylpiperidin-1-yl)pyrimidin-4-yl)amino)isoquinolin-8-yl)-2-methylazetidin-3-yl)-N-isopropylmethanesuIfonamide,
N-((2R,3S)-1-(5-((R)-1-acryloylpiperidin-2-yl)-3-((2-(4-hydroxy-4-methylpiperidin-1-yl)pyrimidin-4-yl)amino)isoquinolin-8-yl)-2-methylazetidin-3-yl)-N-isopropylmethanesuIfonamide,
N-((2R,3S)-1-(5-((S)-1-acryloylpiperidin-2-yl)-3-((2-(4-hydroxy-4-methylpiperidin-1-yl)pyrimidin-4-yl)amino)isoquinolin-8-yl)-2-methylazetidin-3-yl)-N-isopropylmethanesuIfonamide,
N-((2R, 3S)-1-(5-((S)-1-acryloylazepan-2-yl)-3-((2-(4-hydroxy-4-methylpi peridin-1-yl)pyrimidin-4-yl)amino)isoquinolin-8-yl)-2-methylazetidin-3-yl)-N-isopropylmethanesuIfonamide,
N-((2R, 3S)-1-(5-((R)-1-acryloylazepan-2-yl)-3-((2-(4-hydroxy-4-methylpi peridin-1-yl)pyrimidin-4-yl)amino)isoquinolin-8-yl)-2-methylazetidin-3-yl)-N-isopropylmethanesuIfonamide,
N-(2-(3-((2-(4-methoxypiperidin-1-yl)pyrimidin-4-yl)amino)-8-((2R, 3S)-2-methyl-3-(((trifluoromethyl)sulfonyl)methyl)azetidin-1-yl)isoquinolin-5-yl)propan-2-yl)acrylamide,
1-(3-(3-((2-(4-methoxypiperidin-1-yl)pyrimidin-4-yl)amino)-8-((2R, 3S)-2-methyl-3-(((trifluoromethyl)sulfonyl)methyl)azetidin-1-yl)isoquinolin-5-yl)azetidin-1-yl)prop-2-en-1-one,
1-((S)-2-(3-((2-(4-methoxypiperidin-1-yl)pyrimidin-4-yl)amino)-8-((2R,3S)-2-methyl-3-(((trifluoromethyl)sulfonyl)methyl)azetidin-1-yl)isoquinolin-5-yl)azetidin-1-yl)prop-2-en-1-one,
1-((R)-2-(3-((2-(4-methoxypiperidin-1-yl)pyrimidin-4-yl)amino)-8-((2R,3S)-2-methyl-3-(((trifluoromethyl)sulfonyl)methyl)azetidin-1-yl)isoquinolin-5-yl)azetidin-1-yl)prop-2-en-1-one,
1-((S)-2-(3-((2-(4-methoxypiperidin-1-yl)pyrimidin-4-yl)amino)-8-((2R,3S)-2-methyl-3-(((trifluoromethyl)sulfonyl)methyl)azetidin-1-yl)isoquinolin-5-yl)pyrrolidin-1-yl)prop-2-en-1-one,
1-((R)-2-(3-((2-(4-methoxypiperidin-1-yl)pyrimidin-4-yl)amino)-8-((2R,3S)-2-methyl-3-(((trifluoromethyl)sulfonyl)methyl)azetidin-1-yl)isoquinolin-5-yl)pyrrolidin-1-yl)prop-2-en-1-one,
1-((R)-2-(3-((2-(4-methoxypiperidin-1-yl)pyrimidin-4-yl)amino)-8-((2R,3S)-2-methyl-3-(((trifluoromethyl)sulfonyl)methyl)azetidin-1-yl)isoquinolin-5-yl)piperidin-1-yl)prop-2-en-1-one,
1-((S)-2-(3-((2-(4-methoxypiperidin-1-yl)pyrimidin-4-yl)amino)-8-((2R,3S)-2-methyl-3-(((trifluoromethyl)sulfonyl)methyl)azetidin-1-yl)isoquinolin-5-yl)piperidin-1-yl)prop-2-en-1-one,
1-((S)-2-(3-((2-(4-methoxypiperidin-1-yl)pyrimidin-4-yl)amino)-8-((2R,3S)-2-methyl-3-(((trifluoromethyl)sulfonyl)methyl)azetidin-1-yl)isoquinolin-5-yl)azepan-1-yl)prop-2-en-1-one,
1-((R)-2-(3-((2-(4-methoxypiperidin-1-yl)pyrimidin-4-yl)amino)-8-((2R,3S)-2-methyl-3-(((trifluoromethyl)sulfonyl)methyl)azetidin-1-yl)isoquinolin-5-yl)azepan-1-yl)prop-2-en-1-one,
N-(2-(3-((2-(4-methoxypiperidin-1-yl)pyrimidin-4-yl)amino)-8-((2R, 3S)-2-methyl-3-(2-(methylsulfonyl)propan-2-yl)azetidin-1-yl)isoquinolin-5-yl)propan-2-yl)acrylamide,
1-(3-(3-((2-(4-methoxypiperidin-1-yl)pyrimidin-4-yl)amino)-8-((2R, 3S)-2-methyl-3-(2-(methylsulfonyl)propan-2-yl)azetidin-1-yl)isoquinolin-5-yl)azetidin-1-yl)prop-2-en-1-one,
1-((S)-2-(3-((2-(4-methoxypiperidin-1-yl)pyrimidin-4-yl)amino)-8-((2R,3S)-2-methyl-3-(2-(methylsulfonyl)propan-2-yl)azetidin-1-yl)isoquinolin-5-yl)azetidin-1-yl)prop-2-en-1-one,
1-((R)-2-(3-((2-(4-methoxypiperidin-1-yl)pyrimidin-4-yl)amino)-8-((2R,3S)-2-methyl-3-(2-(methylsulfonyl)propan-2-yl)azetidin-1-yl)isoquinolin-5-yl)azetidin-1-yl)prop-2-en-1-one,
1-((S)-2-(3-((2-(4-methoxypiperidin-1-yl)pyrimidin-4-yl)amino)-8-((2R,3S)-2-methyl-3-(2-(methylsulfonyl)propan-2-yl)azetidin-1-yl)isoquinolin-5-yl)pyrrolidin-1-yl)prop-2-en-1-one,
1-((R)-2-(3-((2-(4-methoxypiperidin-1-yl)pyrimidin-4-yl)amino)-8-((2R,3S)-2-methyl-3-(2-(methylsulfonyl)propan-2-yl)azetidin-1-yl)isoquinolin-5-yl)pyrrolidin-1-yl)prop-2-en-1-one,
1-((R)-2-(3-((2-(4-methoxypiperidin-1-yl)pyrimidin-4-yl)amino)-8-((2R,3S)-2-methyl-3-(2-(methylsulfonyl)propan-2-yl)azetidin-1-yl)isoquinolin-5-yl)piperidin-1-yl)prop-2-en-1-one,
1-((S)-2-(3-((2-(4-methoxypiperidin-1-yl)pyrimidin-4-yl)amino)-8-((2R,3S)-2-methyl-3-(2-(methylsulfonyl)propan-2-yl)azetidin-1-yl)isoquinolin-5-yl)piperidin-1-yl)prop-2-en-1-one,
1-((S)-2-(3-((2-(4-methoxypiperidin-1-yl)pyrimidin-4-yl)amino)-8-((2R,3S)-2-methyl-3-(2-(methylsulfonyl)propan-2-yl)azetidin-1-yl)isoquinolin-5-yl)azepan-1-yl)prop-2-en-1-one,
1-((R)-2-(3-((2-(4-methoxypiperidin-1-yl)pyrimidin-4-yl)amino)-8-((2R,3S)-2-methyl-3-(2-(methylsulfonyl)propan-2-yl)azetidin-1-yl)isoquinolin-5-yl)azepan-1-yl)prop-2-en-1-one,
N-(2-(3-((2-(4-methoxypiperidin-1-yl)pyrimidin-4-yl)amino)-8-((3R,4R)-2,2,4-trimethyl-3-((methylsulfonyl)methyl)azetidin-1-yl)isoquinolin-5-yl)propan-2-yl)acrylamide,
1-(3-(3-((2-(4-methoxypiperidin-1-yl)pyrimidin-4-yl)amino)-8-((3R,4R)-2,2,4-trimethyl-3-((methylsulfonyl)methyl)azetidin-1-yl)isoquinolin-5-yl)azetidin-1-yl)prop-2-en-1-one,
1-((S)-2-(3-((2-(4-methoxypiperidin-1-yl)pyrimidin-4-yl)amino)-8-((3R,4R)-2,2,4-trimethyl-3-((methylsulfonyl)methyl)azetidin-1-yl)isoquinolin-5-yl)azetidin-1-yl)prop-2-en-1-one,
1-((R)-2-(3-((2-(4-methoxypiperidin-1-yl)pyrimidin-4-yl)amino)-8-((3R,4R)-2,2,4-trimethyl-3-((methylsulfonyl)methyl)azetidin-1-yl)isoquinolin-5-yl)azetidin-1-yl)prop-2-en-1-one,
1-((S)-2-(3-((2-(4-methoxypiperidin-1-yl)pyrimidin-4-yl)amino)-8-((3R,4R)-2,2,4-trimethyl-3-((methylsulfonyl)methyl)azetidin-1-yl)isoquinolin-5-yl)pyrrolidin-1-yl)prop-2-en-1-one,
1-((R)-2-(3-((2-(4-methoxypiperidin-1-yl)pyrimidin-4-yl)amino)-8-((3R,4R)-2,2,4-trimethyl-3-((methylsulfonyl)methyl)azetidin-1-yl)isoquinolin-5-yl)pyrrolidin-1-yl)prop-2-en-1-one,
1-((R)-2-(3-((2-(4-methoxypiperidin-1-yl)pyrimidin-4-yl)amino)-8-((3R,4R)-2,2,4-trimethyl-3-((methylsulfonyl)methyl)azetidin-1-yl)isoquinolin-5-yl)piperidin-1-yl)prop-2-en-1-one,
1-((S)-2-(3-((2-(4-methoxypiperidin-1-yl)pyrimidin-4-yl)amino)-8-((3R,4R)-2,2,4-trimethyl-3-((methylsulfonyl)methyl)azetidin-1-yl)isoquinolin-5-yl)piperidin-1-yl)prop-2-en-1-one,
1-((S)-2-(3-((2-(4-methoxypiperidin-1-yl)pyrimidin-4-yl)amino)-8-((3R,4R)-2,2,4-trimethyl-3-((methylsulfonyl)methyl)azetidin-1-yl)isoquinolin-5-yl)azepan-1-yl)prop-2-en-1-one,
1-((R)-2-(3-((2-(4-methoxypiperidin-1-yl)pyrimidin-4-yl)amino)-8-((3R,4R)-2,2,4-trimethyl-3-((methylsulfonyl)methyl)azetidin-1-yl)isoquinolin-5-yl)azepan-1-yl)prop-2-en-1-one,
(S)—N-(2-(8-(2,2-di methyl-3-((methylsulfonyl)methyl)azetidin-1-yl)-3-((2-(4-methoxypiperidin-1-yl)pyrimidin-4-yl)amino)isoquinolin-5-yl)propan-2-yl)acrylamide,
(S)-1-(3-(8-(2,2-dimethyl-3-((methylsulfonyl)methyl)azetidin-1-yl)-3-((2-(4-methoxypiperidin-1-yl)pyrimidin-4-yl)amino)isoquinolin-5-yl)azetidin-1-yl)prop-2-en-1-one,
1-((S)-2-(8-((S)-2,2-dimethyl-3-((methylsulfonyl)methyl)azetidin-1-yl)-3-((2-(4-methoxypi peridin-1-yl)pyrimidin-4-yl)amino)isoquinolin-5-yl)azetidin-1-yl)prop-2-en-1-one,
1-((R)-2-(8-((S)-2,2-dimethyl-3-((methylsulfonyl)methyl)azetidin-1-yl)-3-((2-(4-methoxypi peridin-1-yl)pyrimidin-4-yl)amino)isoquinolin-5-yl)azetidin-1-yl)prop-2-en-1-one,
1-((S)-2-(8-((S)-2,2-dimethyl-3-((methylsulfonyl)methyl)azetidin-1-yl)-3-((2-(4-methoxypi peridin-1-yl)pyrimidin-4-yl)amino)isoquinolin-5-yl)pyrrolidin-1-yl)prop-2-en-1-one,
1-((R)-2-(8-((S)-2,2-dimethyl-3-((methylsulfonyl)methyl)azetidin-1-yl)-3-((2-(4-methoxypi peridin-1-yl)pyrimidin-4-yl)amino)isoquinolin-5-yl)pyrrolidin-1-yl)prop-2-en-1-one,
1-((R)-2-(8-((S)-2,2-dimethyl-3-((methylsulfonyl)methyl)azetidin-1-yl)-3-((2-(4-methoxypi peridin-1-yl)pyrimidin-4-yl)amino)isoquinolin-5-yl)piperidin-1-yl)prop-2-en-1-one,
1-((S)-2-(8-((S)-2,2-dimethyl-3-((methylsulfonyl)methyl)azetidin-1-yl)-3-((2-(4-methoxypi peridin-1-yl)pyrimidin-4-yl)amino)isoquinolin-5-yl)piperidin-1-yl)prop-2-en-1-one,
1-((S)-2-(8-((S)-2,2-dimethyl-3-((methylsulfonyl)methyl)azetidin-1-yl)-3-((2-(4-methoxypi peridin-1-yl)pyrimidin-4-yl)amino)isoquinolin-5-yl)azepan-1-yl)prop-2-en-1-one,
1-((R)-2-(8-((S)-2,2-dimethyl-3-((methylsulfonyl)methyl)azetidin-1-yl)-3-((2-(4-methoxypi peridin-1-yl)pyrimidin-4-yl)amino)isoquinolin-5-yl)azepan-1-yl)prop-2-en-1-one,
N-(2-(8-((2R, 3S)-3-(N-isopropylmethylsulfonamido)-2-methylazetidin-1-yl)-3-((2-(4-methoxypiperidin-1-yl)pyrimidin-4-yl)amino)isoquinolin-5-yl)propan-2-yl)acrylamide,
N-((2R,3S)-1-(5-(1-acryloylazetidin-3-yl)-3-((2-(4-methoxypiperidin-1-yl)pyrimidin-4-yl)amino)isoquinolin-8-yl)-2-methylazetidin-3-yl)-N-isopropyl methanesulfonamide,
N-((2R, 3S)-1-(5-((S)-1-acryloylazetidin-2-yl)-3-((2-(4-methoxypiperidin-1-yl)pyrimidin-4-yl)amino)isoquinolin-8-yl)-2-methylazetidin-3-yl)-N-isopropylmethanesuIfonamide,
N-((2R, 3S)-1-(5-((R)-1-acryloylazetidin-2-yl)-3-((2-(4-methoxypiperidin-1-yl)pyrimidin-4-yl)amino)isoquinolin-8-yl)-2-methylazetidin-3-yl)-N-isopropylmethanesuIfonamide,
N-((2R, 3S)-1-(5-((S)-1-acryloylpyrrolidin-2-yl)-3-((2-(4-methoxypiperidin-1-yl)pyrimidin-4-yl)amino)isoquinolin-8-yl)-2-methylazetidin-3-yl)-N-isopropylmethanesuIfonamide,
N-((2R, 3S)-1-(5-((R)-1-acryloylpyrrolidin-2-yl)-3-((2-(4-methoxypiperidin-1-yl)pyrimidin-4-yl)amino)isoquinolin-8-yl)-2-methylazetidin-3-yl)-N-isopropylmethanesuIfonamide,
N-((2R,3S)-1-(5-((R)-1-acryloylpiperidin-2-yl)-3-((2-(4-methoxypiperidin-1-yl)pyrimidin-4-yl)amino)isoquinolin-8-yl)-2-methylazetidin-3-yl)-N-isopropylmethanesuIfonamide,
N-((2R,3S)-1-(5-((S)-1-acryloylpiperidin-2-yl)-3-((2-(4-methoxypiperidin-1-yl)pyrimidin-4-yl)amino)isoquinolin-8-yl)-2-methylazetidin-3-yl)-N-isopropylmethanesuIfonamide,
N-((2R, 3S)-1-(5-((S)-1-acryloylazepan-2-yl)-3-((2-(4-methoxypiperidin-1-yl)pyrimidin-4-yl)amino)isoquinolin-8-yl)-2-methylazetidin-3-yl)-N-isopropylmethanesuIfonamide,
N-((2R, 3S)-1-(5-((R)-1-acryloylazepan-2-yl)-3-((2-(4-methoxypiperidin-1-yl)pyrimidin-4-yl)amino)isoquinolin-8-yl)-2-methylazetidin-3-yl)-N-isopropylmethanesuIfonamide,
1-((S)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((S)-2-(3-((2-((3S,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((R)-2-(3-((2-((3R,4S)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)azetidin-1-yl)prop-2-en-1-one,
1-((R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)azetidin-1-yl)prop-2-en-1-one,
1-((S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)azetidin-1-yl)but-2-yn-1-one,
1-((R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)azetidin-1-yl)but-2-yn-1-one,
1-((S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)but-2-yn-1-one,
1-((R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)but-2-yn-1-one,
1-((S)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((R)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)but-2-yn-1-one,
1-((R)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)but-2-yn-1-one,
1-((S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)piperidin-1-yl)prop-2-en-1-one,
1-((R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)piperidin-1-yl)prop-2-en-1-one,
1-((S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)piperidin-1-yl)but-2-yn-1-one,
1-((R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)piperidin-1-yl)but-2-yn-1-one,
1-((S)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)piperidin-1-yl)prop-2-en-1-one,
1-((R)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)piperidin-1-yl)prop-2-en-1-one,
1-((S)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)piperidin-1-yl)but-2-yn-1-one,
1-((R)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)piperidin-1-yl)but-2-yn-1-one,
N-((1R,2R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclopropyl)acrylamide,
N-((1R,2S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclopropyl)acrylamide,
N-((1 S,2R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclopropyl)acrylamide,
N-((1 S,2S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclopropyl)acrylamide,
N-((1R,2R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclopropyl)but-2-ynamide,
N-((1R,2S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclopropyl)but-2-ynamide,
N-((1 S,2R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclopropyl)but-2-ynamide,
N-((1 S,2S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclopropyl)but-2-ynamide,
N-((1R,2R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclobutyl)acrylamide,
N-((1R,2S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclobutyl)acrylamide,
N-((1 S,2R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclobutyl)acrylamide,
N-((1 S,2S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclobutyl)acrylamide,
N-((1R,2R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclobutyl)but-2-ynamide,
N-((1R,2S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclobutyl)but-2-ynamide,
N-((1 S,2R)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclobutyl)but-2-ynamide,
N-((1 S,2S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)cyclobutyl)but-2-ynamide,
N-((3S,4R)-4-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydrofuran-3-yl)acrylamide,
N-((3R,4R)-4-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydrofuran-3-yl)acrylamide,
N-((3S,4S)-4-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydrofuran-3-yl)acrylamide,
N-((3R,4S)-4-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydrofuran-3-yl)acrylamide,
N-((3S,4R)-4-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydrofuran-3-yl)but-2-ynamide,
N-((3R,4R)-4-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydrofuran-3-yl)but-2-ynamide,
N-((3S,4S)-4-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydrofuran-3-yl)but-2-ynamide,
N-((3R,4S)-4-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydrofuran-3-yl)but-2-ynamide,
N-((3R,4R)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydro-2H-pyran-4-yl)acryIamide,
N-((3S,4R)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydro-2H-pyran-4-yl)acryIamide,
N-((3R,4S)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydro-2H-pyran-4-yl)acryIamide,
N-((3S,4S)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydro-2H-pyran-4-yl)acryIamide,
N-((3R,4R)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydro-2H-pyran-4-yl)but-2-ynamide,
N-((3S,4R)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydro-2H-pyran-4-yl)but-2-ynamide,
N-((3R,4S)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydro-2H-pyran-4-yl)but-2-ynamide,
N-((3S,4S)-3-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)tetrahydro-2H-pyran-4-yl)but-2-ynamide,
1-((S)-2-(3-((2-((3S,4R)-3-fluoro-4-methoxypiperidin-1-yl)pyrimidin-4-yl)amino)-8-((2R,3S)-2-methyl-3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)but-2-yn-1-one,
2-fluoro-1-((S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
(E)-4-(dimethylamino)-1-((S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)pyrrolidin-1-yl)but-2-en-1-one,
1-((S)-2-(3-((2-((3S,4R)-3-fluoro-4-methoxypiperidin-1-yl)pyrimidin-4-yl)amino)-8-(3-((methylsulfonyl)methyl)azetidin-1-yl)isoquinolin-5-yl)-2-methyl pyrrolidin-1-yl)prop-2-en-1-one,
1-((S)-2-(3-((2-((2S,4S,5R)-5-fluoro-4-methoxy-2-methylpiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((2R,4S,5R)-5-fluoro-4-methoxy-2-methylpiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((2S,4R,5S)-5-fluoro-4-methoxy-2-methylpiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((2R,4R,5S)-5-fluoro-4-methoxy-2-methylpiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-isopropoxypi peridin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-4-(tert-butoxy)-3-fluoropiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-(trifluoromethoxy)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-(2-hydroxyethoxy)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-(2-methoxyethoxy)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-(2-(methyl amino)ethoxy)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-(3-morpholinopropoxy)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-((tetrahydro-2H-pyran-4-yl)oxy)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-((4-hydroxy-4-methylcyclohexyl)oxy)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-((1-(tetrahydro-2H-pyran-4-yl)piperidin-4-yl)oxy)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-((1-(6-(trifluoromethyl)pyridin-2-yl)piperidin-4-yl)oxy)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
5-(4-(((3S,4R)-1-(4-((5-((S)-1-acryloylpyrrolidin-2-yl)-8-(3-((methylsulfonyl)methyl)azetidin-1-yl)isoquinolin-3-yl)amino)pyrimidin-2-yl)-3-fluoropiperidin-4-yl)oxy)piperidin-1-yl)-2-(2,6-dioxopiperidin-3-yl)isoindoline-1,3-dione,
1-((S)-2-(3-((2-((3S,4R)-3-fluoro-4-morpholinopiperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3'S,4′R)-3′-fluoro-4-hydroxy-4-methyl-[1,4′-bipiperidin]-1′-yl)pyrimidin-4-yl)amino)-8-(3-((methylsulfonyl)methyl)azetidin-1-yl)isoquinolin-5-yl)pyrrolidin-1-yl)prop-2-en-1-one,
4R)-3-fluoro-4-(4-(tetrahydro-2H-pyran-4-yl)piperazin-1-yl)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-(4-(6-(trifluoromethyl)pyridin-2-yl)piperazin-1-yl)piperidin-1-yl)pyrimidin-4-yl)amino)-8-(3-((methylsulfonyl)methyl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-((S)-3-((methylsulfonyl)methyl)pyrrolidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-((R)-3-((methylsulfonyl)methyl)pyrrolidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(4-((methylsulfonyl)methyl)piperidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-((R)-3-((methylsulfonyl)methyl)piperidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-((S)-3-((methylsulfonyl)methyl)piperidin-1-yl)isoquinolin-5-yl)pyrrolidin-1-yl)prop-2-en-1-one,
1-((S)-2-(8-(3-((S)-1,1-dioxidotetrahydrothiophen-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((R)-1,1-dioxidotetrahydrothiophen-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((R)-1,1-dioxidotetrahydro-2H-thiopyran-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((S)-1,1-dioxidotetrahydro-2H-thiopyran-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((S)-4,4-dioxido-1,4-oxathian-3-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((R)-4,4-dioxido-1,4-oxathian-3-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-((2R,4S)-4-hydroxy-4-methyl-1,1-dioxidotetrahydro-2H-thiopyran-2-yl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-((2R,4R)-4-hydroxy-4-methyl-1,1-dioxidotetrahydro-2H-thiopyran-2-yl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-((2S,4S)-4-hydroxy-4-methyl-1,1-dioxidotetrahydro-2H-thiopyran-2-yl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-((2S,4R)-4-hydroxy-4-methyl-1,1-dioxidotetrahydro-2H-thiopyran-2-yl)azetidin-1-yl)isoquinolin-5-yl)pyrrolidin-1-yl)prop-2-en-1-one,
1-((S)-2-(8-(3-((S)-1,1-dioxido-4-(tetrahydro-2H-pyran-4-yl)thiomorpholin-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((R)-1,1-dioxido-4-(tetrahydro-2H-pyran-4-yl)thiomorpholin-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((R)-1,1-dioxido-4-(2-(trifluoromethyl)pyridin-4-yl)thiomorpholin-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((S)-1,1-dioxido-4-(2-(trifluoromethyl)pyridin-4-yl)thiomorpholin-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
5-((R)-2-(1-(5-((S)-1-acryloylpyrrolidin-2-yl)-3-((2-((3S,4R)-3-fluoro-4-methoxypiperidin-1-yl)pyrimidin-4-yl)amino)isoquinolin-8-yl)azetidin-3-yl)-1,1-dioxidothiomorpholino)-2-(2,6-dioxopiperidin-3-yl)isoindoline-1,3-dione,
5-((S)-2-(1-(5-((S)-1-acryloylpyrrolidin-2-yl)-3-((2-((3S,4R)-3-fluoro-4-methoxypiperidin-1-yl)pyrimidin-4-yl)amino)isoquinolin-8-yl)azetidin-3-yl)-1,1-dioxidothiomorpholino)-2-(2,6-dioxopiperidin-3-yl)isoindoline-1,3-dione,
1-((S)-2-(3-((2-((3S,4R)-3-fluoro-4-methoxypiperidin-1-yl)pyrimidin-4-yl)amino)-8-(3-((2R,6S)-6-methyl-1,1-dioxidotetrahydro-2H-thiopyran-2-yl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-((2R,6R)-6-methyl-1-dioxidotetrahydro-2H-thiopyran-2-yl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-((2S,6S)-6-methyl-1,1-dioxidotetrahydro-2H-thiopyran-2-yl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-((2S,6R)-6-methyl-1-dioxidotetrahydro-2H-thiopyran-2-yl)azetidin-1-yl)isoquinolin-5-yl)pyrrolidin-1-yl)prop-2-en-1-one,
1-((S)-2-(8-(3-((R)-6,6-dimethyl-1,1-dioxidotetrahydro-2H-thiopyran-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((S)-6,6-dimethyl-1,1-dioxidotetrahydro-2H-thiopyran-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-(2-(isopropylsulfonyl)propan-2-yl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-(3-(1-(isopropylsulfonyl)cyclobutyl)azetidin-1-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-((1 S,2S,6R)-2-(methylsulfonyl)-7-azabicyclo[4.2.0]octan-7-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-((1 S,2R,6R)-2-(methylsulfonyl)-7-azabicyclo[4.2.0]octan-7-yl)isoquinolin-5-yl)pyrrolidin-1-yl)prop-2-en-1-one,
1-((R)-2-(8-((4aR,7aS,9aR,9bR)-1,1-dioxidodecahydrothiopyrano[2′,3′:3,4]cyclohepta[1,2-b]azet-8(2H)-yl)-3-((2-((3R,4S)-3-fluoro-4-methoxypiperidin-1-yl)pyrimidin-4-yl)amino)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-((S)-5-(methylsulfonyl)-2-azaspiro[3.4]octan-2-yl)isoquinolin-5-yl)pyrrolidin-1-yl)prop-2-en-1-one,
1-((S)-2-(3-((2-((3S,4R)-3-fluoro-4-methoxypiperidin-1-yl)pyrimidin-4-yl)amino)-8-((R)-5-(methylsulfonyl)-2-azaspiro[3.4]octan-2-yl)isoquinolin-5-yl)pyrrolidin-1-yl)prop-2-en-1-one. 1-((R)-2-(8-(3-(1,1-dioxido-1,2-thiazinan-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-(1,1-dioxido-1,2-thiazinan-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-(4-(1,1-dioxido-1,2-thiazinan-2-yl)piperidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(4-(1,1-dioxido-1,2-thiazinan-2-yl)piperidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-(4-((S)-1,1-dioxidotetrahydro-2H-thiopyran-2-yl)piperazin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-(4-((R)-1,1-dioxidotetrahydro-2H-thiopyran-2-yl)piperazin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(4-((S)-1,1-dioxidotetrahydro-2H-thiopyran-2-yl)piperazin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(4-((R)-1,1-dioxidotetrahydro-2H-thiopyran-2-yl)piperazin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-(4-((R)-1,1-dioxidotetrahydrothiophen-2-yl)piperidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-(4-((S)-1,1-dioxidotetrahydrothiophen-2-yl)piperidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(4-((R)-1,1-dioxidotetrahydrothiophen-2-yl)piperidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(4-((S)-1,1-dioxidotetrahydrothiophen-2-yl)piperidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-(3-((R)-1,1-dioxidotetrahydrothiophen-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-(3-((S)-1,1-dioxidotetrahydrothiophen-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((R)-1,1-dioxidotetrahydrothiophen-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-(3-((S)-1,1-dioxidotetrahydrothiophen-2-yl)azetidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-((R)-3-((R)-1,1-dioxidotetrahydro-2H-thiopyran-2-yl)pyrrolidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-((S)-3-((R)-1,1-dioxidotetrahydro-2H-thiopyran-2-yl)pyrrolidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-((R)-3-((S)-1,1-dioxidotetrahydro-2H-thiopyran-2-yl)pyrrolidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(8-((S)-3-((S)-1,1-dioxidotetrahydro-2H-thiopyran-2-yl)pyrrolidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-((R)-3-((R)-1,1-dioxidotetrahydro-2H-thiopyran-2-yl)pyrrolidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-((S)-3-((R)-1,1-dioxidotetrahydro-2H-thiopyran-2-yl)pyrrolidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-((R)-3-((S)-1,1-dioxidotetrahydro-2H-thiopyran-2-yl)pyrrolidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-((S)-3-((S)-1,1-dioxidotetrahydro-2H-thiopyran-2-yl)pyrrolidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((S)-2-(8-((R)-3-((S)-1,1-dioxidotetrahydro-2H-thiopyran-2-yl)piperidin-1-yl)-3-((2-((3S,4R)-3-fluoro-4-methoxypiperidin-1-yl)pyrimidin-4-yl)amino)isoquinolin-5-yl)pyrrolidin-1-yl)prop-2-en-1-one,
1-((R)-2-(2-((2-((3S,4R)-3-fluoro-4-methoxypiperidin-1-yl)pyridin-4-yl)amino)-5-((2R,3S)-2-methyl-3-((methylsulfonyl)methyl)azetidin-1-yl)quinazolin-8-yl)pyrrolidin-1-yl)prop-2-en-1-one,
1-((S)-2-(2-((2-((3S,4R)-3-fluoro-4-methoxypiperidin-1-yl)pyridin-4-yl)amino)-5-((2R,3S)-2-methyl-3-((methylsulfonyl)methyl)azetidin-1-yl)quinazolin-8-yl)pyrrolidin-1-yl)prop-2-en-1-one,
1-(2-(6-((2-((3S,4R)-3-fluoro-4-methoxypiperidin-1-yl)pyrimidin-4-yl)amino)-1-(3-((methylsulfonyl)methyl)azetidin-1-yl)-2,7-naphthyridin-4-yl)pyrrolidin-1-yl)prop-2-en-1-one,
1-((S)-2-(6-((2-((3S,4R)-3-fluoro-4-methoxypiperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-methoxypiperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)but-2-yn-1-one,
2-fluoro-1-(2-(6-((2-((3S,4R)-3-fluoro-4-methoxypiperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
(E)-4-(dimethylamino)-1-(2-(6-((2-((3S,4R)-3-fluoro-4-methoxypiperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)but-2-en-1-one,
1-(2-(6-((2-((3S,4R)-3-fluoro-4-methoxypiperidin-1-yl)pyrimidin-4-yl)amino)-1-((2R,3S)-2-methyl-3-((methylsulfonyl)methyl)azetidin-1-yl)-2,7-naphthyridin-4-yl)-2-methylpyrrolidin-1-yl)prop-2-en-1-one,
1-(2-(6-((2-((2S,4R,5S)-5-fluoro-4-methoxy-2-methylpiperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((2R,4R,5S)-5-fluoro-4-methoxy-2-methylpiperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-isopropoxypiperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-4-(tert-butoxy)-3-fluoropiperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-(trifluoromethoxy)piperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-(2-hydroxyethoxy)piperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-(2-methoxyethoxy)piperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-(2-(methylamino)ethoxy)piperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-(3-morpholinopropoxy)piperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-((tetrahydro-2H-pyran-4-yl)oxy)piperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-((4-hydroxy-4-methylcyclohexyl)oxy)piperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-((S)-2-(6-((2-((3S,4R)-3-fluoro-4-((1-(tetrahydro-2H-pyran-4-yl)piperidin-4-yl)oxy)piperidin-1-yl)pyrimidin-4-yl)amino)-1-((2R, 3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-((1-(6-(trifluoromethyl)pyridin-2-yl)piperidin-4-yl)oxy)piperidin-1-yl)pyrimidin-4-yl)amino)-1-((2R, 3S)-2-methyl-3-((methylsulfonyl)methyl)azetidin-1-yl)-2,7-naphthyridin-4-yl)pyrrolidin-1-yl)prop-2-en-1-one,
5-(4-(((3S,4R)-1-(4-((5-(1-acryloylpyrrolidin-2-yl)-8-((2R,3S)-2-methyl-3-((methylsulfonyl)methyl)azetidin-1-yl)-2,7-naphthyridin-3-yl)amino)pyrimidin-2-yl)-3-fluoropiperidin-4-yl)oxy)piperidin-1-yl)-2-(2,6-dioxopiperidin-3-yl)isoindoline-1,3-dione,
1-(2-(6-((2-((3S,4R)-3-fluoro-4-morpholinopiperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3'S,4′R)-3′-fluoro-4-hydroxy-4-methyl-[1,4′-bipiperidin]-1′-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-(4-(tetrahydro-2H-pyran-4-yl)piperazin-1-yl)piperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-(2-(6-((2-((3S,4R)-3-fluoro-4-(4-(6-(trifluoromethyl)pyridin-2-yl)piperazin-1-yl)piperidin-1-yl)pyrimidin-4-yl)amino)-1-((2R,3S)-2-methyl-3-((methylsulfonyl)methyl)azetidin-1-yl)-2,7-naphthyridin-4-yl)pyrrolidin-1-yl)prop-2-en-1-one,
1-((R)-2-(1-(4-((R)-1,1-dioxidotetrahydro-2H-thiopyran-2-yl)piperidin-1-yl)-6-((2-((3S,4R)-3-fluoro-4-methoxypiperidin-1-yl)pyrimidin-4-yl)amino)-2,7-naphthyridin-4-yl)pyrrolidin-1-yl)prop-2-en-1-one,
1-((R)-2-(1-(4-((S)-1,1-dioxidotetrahydro-2H-thiopyran-2-yl)piperidin-1-yl)-6-((2-((3S,4R)-3-fluoro-4-methoxypiperidin-1-yl)pyrimidin-4-yl)amino)-2,7-naphthyridin-4-yl)pyrrolidin-1-yl)prop-2-en-1-one,
1-(2-(1-(3-(4,4-dioxido-1,4-oxathian-3-yl)-2-methylazetidin-1-yl)-6-((2-((3S,4R)-3-fluoro-4-methoxypiperidin-1-yl)pyrimidin-4-yl)amino)-2,7-naphthyridin-4-yl)pyrrolidin-1-yl)prop-2-en-1-one,
1-(2-(6-((2-((3S,4R)-3-fluoro-4-methoxypiperidin-1-yl)pyrimidin-4-yl)amino)-1-(3-(4-hydroxy-4-methyl-1,1-dioxidotetrahydro-2H-thiopyran-2-yl)-2-methylazetidin-1-yl)-2,7-naphthyridin-4-yl)pyrrolidin-1-yl)prop-2-en-1-one,
1-(2-(1-(3-(1,1-dioxido-4-(tetrahydro-2H-pyran-4-yl)thiomorpholin-2-yl)-2-methylazetidin-1-yl)-6-((2-((3S,4R)-3-fluoro-4-methoxypiperidin-1-yl)pyrimidin-4-yl)amino)-2,7-naphthyridin-4-yl)pyrrolidin-1-yl)prop-2-en-1-one,
1-(2-(1-(3-(1,1-dioxido-4-(2-(trifluoromethyl)pyridin-4-yl)thiomorpholin-2-yl)-2-methylazetidin-1-yl)-6-((2-((3S,4R)-3-fluoro-4-methoxypiperidin-1-yl)pyrimidin-4-yl)amino)-2,7-naphthyridin-4-yl)pyrrolidin-1-yl)prop-2-en-1-one,
5-(2-(1-(4-(1-acryloylpyrrolidin-2-yl)-6-((2-((3S,4R)-3-fluoro-4-methoxypiperidin-1-yl)pyrimidin-4-yl)amino)-2,7-naphthyridin-1-yl)-2-methylazetidin-3-yl)-1,1-dioxidothiomorpholino)-2-(2,6-dioxopiperidin-3-yl)isoindoline-1,3-dione,
1-(2-(6-((2-((3S,4R)-3-fluoro-4-methoxypiperidin-1-yl)pyrimidin-4-yl)amino)-1-(2-methyl-3-(6-methyl-1,1-dioxidotetrahydro-2H-thiopyran-2-yl)azetidin-1-yl)-2,7-naphthyridin-4-yl)pyrrolidin-1-yl)prop-2-en-1-one,
1-(2-(1-(3-(6,6-dimethyl-1,1-dioxidotetrahydro-2H-thiopyran-2-yl)-2-methylazetidin-1-yl)-6-((2-((3S,4R)-3-fluoro-4-methoxypiperidin-1-yl)pyrimidin-4-yl)amino)-2,7-naphthyridin-4-yl)pyrrolidin-1-yl)prop-2-en-1-one,
1-(2-(1-(3-(4,4-dioxido-1,4-oxathian-3-yl)azetidin-1-yl)-6-((2-((3S,4R)-3-fluoro-4-methoxypiperidin-1-yl)pyrimidin-4-yl)amino)-2,7-naphthyridin-4-yl)pyrrolidin-1-yl)prop-2-en-1-one,
1-(2-(6-((2-((3S,4R)-3-fluoro-4-methoxypiperidin-1-yl)pyrimidin-4-yl)amino)-1-(3-(4-hydroxy-4-methyl-1,1-dioxidotetrahydro-2H-thiopyran-2-yl)azetidin-1-yl)-2,7-naphthyridin-4-yl)pyrrolidin-1-yl)prop-2-en-1-one,
1-(2-(1-(3-(1,1-dioxido-4-(tetrahydro-2H-pyran-4-yl)thiomorpholin-2-yl)azetidin-1-yl)-6-((2-((3S,4R)-3-fluoro-4-methoxypiperidin-1-yl)pyrimidin-4-yl)amino)-2,7-naphthyridin-4-yl)pyrrolidin-1-yl)prop-2-en-1-one,
1-(2-(1-(3-(1,1-dioxido-4-(2-(trifluoromethyl)pyridin-4-yl)thiomorpholin-2-yl)azetidin-1-yl)-6-((2-((3S,4R)-3-fluoro-4-methoxypiperidin-1-yl)pyrimidin-4-yl)amino)-2,7-naphthyridin-4-yl)pyrrolidin-1-yl)prop-2-en-1-one,
1-(2-(6-((2-((3S,4R)-3-fluoro-4-methoxypiperidin-1-yl)pyrimidin-4-yl)amino)-1-(3-(6-methyl-1,1-dioxidotetrahydro-2H-thiopyran-2-yl)azetidin-1-yl)-2,7-naphthyridin-4-yl)pyrrolidin-1-yl)prop-2-en-1-one,
1-(2-(1-(3-(6,6-dimethyl-1,1-dioxidotetrahydro-2H-thiopyran-2-yl)azetidin-1-yl)-6-((2-((3S,4R)-3-fluoro-4-methoxypiperidin-1-yl)pyrimidin-4-yl)amino)-2,7-naphthyridin-4-yl)pyrrolidin-1-yl)prop-2-en-1-one,
5-(2-(1-(4-(1-acryloylpyrrolidin-2-yl)-6-((2-((3S,4R)-3-fluoro-4-methoxypiperidin-1-yl)pyrimidin-4-yl)amino)-2,7-naphthyridin-1-yl)azetidin-3-yl)-1,1-dioxidothiomorpholino)-2-(2,6-dioxopiperidin-3-yl)isoindoline-1,3-dione,
1-((R)-2-(2-((2-((3S,4R)-3-fluoro-4-methoxypiperidin-1-yl)pyridin-4-yl)amino)-5-((2R,3S)-2-methyl-3-((methylsulfonyl)methyl)azetidin-1-yl)pyrido[4,3-d]pyri midin-8-yl)pyrrolidin-1-yl)prop-2-en-1-one,
1-((S)-2-(2-((2-((3S,4R)-3-fluoro-4-methoxypiperidin-1-yl)pyridin-4-yl)amino)-5-((2R,3S)-2-methyl-3-((methylsulfonyl)methyl)azetidin-1-yl)pyrido[4,3-d]pyri midin-8-yl)pyrrolidin-1-yl)prop-2-en-1-one,
1-(2-(7-((2-((3S,4R)-3-fluoro-4-methoxypiperidin-1-yl)pyrimidin-4-yl)amino)-4-(3-((methylsulfonyl)methyl)azetidin-1-yl)-2,6-naphthyridin-1-yl)pyrrolidin-1-yl)prop-2-en-1-one,
1-((S)-2-(7-((2-((3S,4R)-3-fluoro-4-methoxypiperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-methoxypiperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)but-2-yn-1-one,
2-fluoro-1-(2-(7-((2-((3S,4R)-3-fluoro-4-methoxypiperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
(E)-4-(dimethylamino)-1-(2-(7-((2-((3S,4R)-3-fluoro-4-methoxypiperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)but-2-en-1-one,
1-(2-(7-((2-((3S,4R)-3-fluoro-4-methoxypiperidin-1-yl)pyrimidin-4-yl)amino)-4-((2R,3S)-2-methyl-3-((methylsulfonyl)methyl)azetidin-1-yl)-2,6-naphthyridin-1-yl)-2-methylpyrrolidin-1-yl)prop-2-en-1-one,
1-(2-(7-((2-((2S,4R,5S)-5-fluoro-4-methoxy-2-methylpiperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((2R,4R,5S)-5-fluoro-4-methoxy-2-methylpiperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-isopropoxypiperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-4-(tert-butoxy)-3-fluoropiperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-(trifluoromethoxy)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-(2-hydroxyethoxy)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-(2-methoxyethoxy)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-(2-(methylamino)ethoxy)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-(3-morpholinopropoxy)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-((tetrahydro-2H-pyran-4-yl)oxy)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-((4-hydroxy-4-methylcyclohexyl)oxy)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-((S)-2-(7-((2-((3S,4R)-3-fluoro-4-((1-(tetrahydro-2H-pyran-4-yl)piperidin-4-yl)oxy)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-((1-(6-(trifluoromethyl)pyridin-2-yl)piperidin-4-yl)oxy)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
5-(4-(((3S,4R)-1-(4-((5-(1-acryloylpyrrolidin-2-yl)-8-((2R,3S)-2-methyl-3-((methylsulfonyl)methyl)azetidin-1-yl)-2,6-naphthyridin-3-yl)amino)pyrimidin-2-yl)-3-fluoropiperidin-4-yl)oxy)piperidin-1-yl)-2-(2,6-dioxopiperidin-3-yl)isoindoline-1,3-dione,
1-(2-(7-((2-((3S,4R)-3-fluoro-4-morpholinopiperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3'S,4′R)-3′-fluoro-4-hydroxy-4-methyl-[1,4′-bipiperidin]-1′-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-(4-(tetrahydro-2H-pyran-4-yl)piperazin-1-yl)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(7-((2-((3S,4R)-3-fluoro-4-(4-(6-(trifluoromethyl)pyridin-2-yl)piperazin-1-yl)piperidin-1-yl)pyrimidin-4-yl)amino)-4-((2R,3S)-2-methyl-3-((methylsulfonyl)methyl)azetidin-1-yl)-2,6-naphthyridin-1-yl)pyrrolidin-1-yl)prop-2-en-1-one,
1-(2-(4-(3-(4,4-dioxido-1,4-oxathian-3-yl)-2-methylazetidin-1-yl)-7-((2-((3S,4R)-3-fluoro-4-methoxypiperidin-1-yl)pyrimidin-4-yl)amino)-2,6-naphthyridin-1-yl)pyrrolidin-1-yl)prop-2-en-1-one,
1-(2-(7-((2-((3S,4R)-3-fluoro-4-methoxypiperidin-1-yl)pyrimidin-4-yl)amino)-4-(3-(4-hydroxy-4-methyl-1,1-dioxidotetrahydro-2H-thiopyran-2-yl)-2-methylazetidin-1-yl)-2,6-naphthyridin-1-yl)pyrrolidin-1-yl)prop-2-en-1-one,
1-(2-(4-(3-(1,1-dioxido-4-(tetrahydro-2H-pyran-4-yl)thiomorpholin-2-yl)-2-methylazetidin-1-yl)-7-((2-((3S,4R)-3-fluoro-4-methoxypiperidin-1-yl)pyrimidin-4-yl)amino)-2,6-naphthyridin-1-yl)pyrrolidin-1-yl)prop-2-en-1-one,
1-(2-(4-(3-(1,1-dioxido-4-(2-(trifluoromethyl)pyridin-4-yl)thiomorpholin-2-yl)-2-methylazetidin-1-yl)-7-((2-((3S,4R)-3-fluoro-4-methoxypiperidin-1-yl)pyrimidin-4-yl)amino)-2,6-naphthyridin-1-yl)pyrrolidin-1-yl)prop-2-en-1-one,
5-(2-(1-(1-(1-acryloylpyrrolidin-2-yl)-7-((2-((3S,4R)-3-fluoro-4-methoxypiperidin-1-yl)pyrimidin-4-yl)amino)-2,6-naphthyridin-4-yl)-2-methylazetidin-3-yl)-1,1-dioxidothiomorpholino)-2-(2,6-dioxopiperidin-3-yl)isoindoline-1,3-dione,
1-(2-(7-((2-((3S,4R)-3-fluoro-4-methoxypiperidin-1-yl)pyrimidin-4-yl)amino)-4-(2-methyl-3-(6-methyl-1,1-dioxidotetrahydro-2H-thiopyran-2-yl)azetidin-1-yl)-2,6-naphthyridin-1-yl)pyrrolidin-1-yl)prop-2-en-1-one,
1-(2-(4-(3-(6,6-di methyl-1,1-dioxidotetrahydro-2H-thiopyran-2-yl)-2-methylazetidin-1-yl)-7-((2-((3S,4R)-3-fluoro-4-methoxypiperidin-1-yl)pyrimidin-4-yl)amino)-2,6-naphthyridin-1-yl)pyrrolidin-1-yl)prop-2-en-1-one,
1-(2-(4-(3-(4,4-dioxido-1,4-oxathian-3-yl)azetidin-1-yl)-7-((2-((3S,4R)-3-fluoro-4-methoxypiperidin-1-yl)pyrimidin-4-yl)amino)-2,6-naphthyridin-1-yl)pyrrolidin-1-yl)prop-2-en-1-one,
1-(2-(7-((2-((3S,4R)-3-fluoro-4-methoxypiperidin-1-yl)pyrimidin-4-yl)amino)-4-(3-(4-hydroxy-4-methyl-1,1-dioxidotetrahydro-2H-thiopyran-2-yl)azetidin-1-yl)-2,6-naphthyridin-1-yl)pyrrolidin-1-yl)prop-2-en-1-one,
1-(2-(4-(3-(1,1-dioxido-4-(tetrahydro-2H-pyran-4-yl)thiomorpholin-2-yl)azetidin-1-yl)-7-((2-((3S,4R)-3-fluoro-4-methoxypiperidin-1-yl)pyrimidin-4-yl)amino)-2,6-naphthyridin-1-yl)pyrrolidin-1-yl)prop-2-en-1-one,
1-(2-(4-(3-(1,1-dioxido-4-(2-(trifluoromethyl)pyridin-4-yl)thiomorpholin-2-yl)azetidin-1-yl)-7-((2-((3S,4R)-3-fluoro-4-methoxypiperidin-1-yl)pyrimidin-4-yl)amino)-2,6-naphthyridin-1-yl)pyrrolidin-1-yl)prop-2-en-1-one,
1-(2-(7-((2-((3S,4R)-3-fluoro-4-methoxypiperidin-1-yl)pyrimidin-4-yl)amino)-4-(3-(6-methyl-1,1-dioxidotetrahydro-2H-thiopyran-2-yl)azetidin-1-yl)-2,6-naphthyridin-1-yl)pyrrolidin-1-yl)prop-2-en-1-one,
1-(2-(4-(3-(6,6-dimethyl-1,1-dioxidotetrahydro-2H-thiopyran-2-yl)azetidin-1-yl)-7-((2-((3S,4R)-3-fluoro-4-methoxypiperidin-1-yl)pyrimidin-4-yl)amino)-2,6-naphthyridin-1-yl)pyrrolidin-1-yl)prop-2-en-1-one,
5-(2-(1-(1-(1-acryloylpyrrolidin-2-yl)-7-((2-((3S,4R)-3-fluoro-4-methoxypiperidin-1-yl)pyrimidin-4-yl)amino)-2,6-naphthyridin-4-yl)azetidin-3-yl)-1,1-dioxidothiomorpholino)-2-(2,6-dioxopiperidin-3-yl)isoindoline-1,3-dione,
1-((R)-2-(2-((2-((3S,4R)-3-fluoro-4-methoxypiperidin-1-yl)pyridin-4-yl)amino)-5-((2R,3S)-2-methyl-3-((methylsulfonyl)methyl)azetidin-1-yl)pyrido[3,4-d]pyri midin-8-yl)pyrrolidin-1-yl)prop-2-en-1-one,
1-((S)-2-(2-((2-((3S,4R)-3-fluoro-4-methoxypiperidin-1-yl)pyridin-4-yl)amino)-5-((2R,3S)-2-methyl-3-((methylsulfonyl)methyl)azetidin-1-yl)pyrido[3,4-d]pyrimidin-8-yl)pyrrolidin-1-yl)prop-2-en-1-one
1-(2-(7-((2-((3S,4R)-3-fluoro-4-methoxypiperidin-1-yl)pyrimidin-4-yl)amino)-4-(3-((methylsulfonyl)methyl)azetidin-1-yl)pyrido[3,4-d]pyridazin-1-yl)pyrrolidin-1-yl)prop-2-en-1-one,
1-((S)-2-(7-((2-((3S,4R)-3-fluoro-4-methoxypiperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-methoxypiperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)but-2-yn-1-one,
2-fluoro-1-(2-(7-((2-((3S,4R)-3-fluoro-4-methoxypiperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
(E)-4-(dimethylamino)-1-(2-(7-((2-((3S,4R)-3-fluoro-4-methoxypiperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)but-2-en-1-one,
1-(2-(7-((2-((3S,4R)-3-fluoro-4-methoxypiperidin-1-yl)pyrimidin-4-yl)amino)-4-((2R,3S)-2-methyl-3-((methylsulfonyl)methyl)azetidin-1-yl)pyrido[3,4-d]pyridazin-1-yl)-2-methylpyrrolidin-1-yl)prop-2-en-1-one,
1-(2-(7-((2-((2S,4R,5S)-5-fluoro-4-methoxy-2-methylpiperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((2R,4R,5S)-5-fluoro-4-methoxy-2-methylpiperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-isopropoxypiperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-4-(tert-butoxy)-3-fluoropiperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-(trifluoromethoxy)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-(2-hydroxyethoxy)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-(2-methoxyethoxy)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-(2-(methylamino)ethoxy)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-(3-morpholinopropoxy)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-((tetrahydro-2H-pyran-4-yl)oxy)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-((4-hydroxy-4-methylcyclohexyl)oxy)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-((S)-2-(7-((2-((3S,4R)-3-fluoro-4-((1-(tetrahydro-2H-pyran-4-yl)piperidin-4-yl)oxy)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-((1-(6-(trifluoromethyl)pyridin-2-yl)piperidin-4-yl)oxy)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
5-(4-(((3S,4R)-1-(4-((1-(1-acryloylpyrrolidin-2-yl)-4-((2R,3S)-2-methyl-3-((methylsulfonyl)methyl)azetidin-1-yl)pyrido[3,4-d]pyridazin-7-yl)amino)pyrimidin-2-yl)-3-fluoropiperidin-4-yl)oxy)piperidin-1-yl)-2-(2,6-dioxopiperidin-3-yl)isoindoline-1,3-dione,
1-(2-(7-((2-((3S,4R)-3-fluoro-4-morpholinopiperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3'S,4′R)-3′-fluoro-4-hydroxy-4-methyl-[1,4′-bipiperidin]-1′-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-(4-(tetrahydro-2H-pyran-4-yl)piperazin-1-yl)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(7-((2-((3S,4R)-3-fluoro-4-(4-(6-(trifluoromethyl)pyridin-2-yl)piperazin-1-yl)piperidin-1-yl)pyrimidin-4-yl)amino)-4-((2R,3S)-2-methyl-3-((methylsulfonyl)methyl)azetidin-1-yl)pyrido[3,4-d]pyridazin-1-yl)pyrrolidin-1-yl)prop-2-en-1-one,
1-(2-(4-(3-(4,4-dioxido-1,4-oxathian-3-yl)-2-methylazetidin-1-yl)-7-((2-((3S,4R)-3-fluoro-4-methoxypiperidin-1-yl)pyrimidin-4-yl)amino)pyrido[3,4-d]pyridazin-1-yl)pyrrolidin-1-yl)prop-2-en-1-one,
1-(2-(7-((2-((3S,4R)-3-fluoro-4-methoxypiperidin-1-yl)pyrimidin-4-yl)amino)-4-(3-(4-hydroxy-4-methyl-1,1-dioxidotetrahydro-2H-thiopyran-2-yl)-2-methylazetidin-1-yl)pyrido[3,4-d]pyridazin-1-yl)pyrrolidin-1-yl)prop-2-en-1-one,
1-(2-(4-(3-(1,1-dioxido-4-(tetrahydro-2H-pyran-4-yl)thiomorpholin-2-yl)-2-methylazetidin-1-yl)-7-((2-((3S,4R)-3-fluoro-4-methoxypiperidin-1-yl)pyrimidin-4-yl)amino)pyrido[3,4-d]pyridazin-1-yl)pyrrolidin-1-yl)prop-2-en-1-one,
1-(2-(4-(3-(1,1-dioxido-4-(2-(trifluoromethyl)pyridin-4-yl)thiomorpholin-2-yl)-2-methylazetidin-1-yl)-7-((2-((3S,4R)-3-fluoro-4-methoxypiperidin-1-yl)pyrimidin-4-yl)amino)pyrido[3,4-d]pyridazin-1-yl)pyrrolidin-1-yl)prop-2-en-1-one,
5-(2-(1-(1-(1-acryloylpyrrolidin-2-yl)-7-((2-((3S,4R)-3-fluoro-4-methoxypiperidin-1-yl)pyrimidin-4-yl)amino)pyrido[3,4-d]pyridazin-4-yl)-2-methylazetidin-3-yl)-1,1-dioxidothiomorpholino)-2-(2,6-dioxopiperidin-3-yl)isoindoline-1,3-dione,
1-(2-(7-((2-((3S,4R)-3-fluoro-4-methoxypiperidin-1-yl)pyrimidin-4-yl)amino)-4-(2-methyl-3-(6-methyl-1,1-dioxidotetrahydro-2H-thiopyran-2-yl)azetidin-1-yl)pyrido[3,4-d]pyridazin-1-yl)pyrrolidin-1-yl)prop-2-en-1-one,
1-(2-(4-(3-(6,6-dimethyl-1,1-dioxidotetrahydro-2H-thiopyran-2-yl)-2-methylazetidin-1-yl)-7-((2-((3S,4R)-3-fluoro-4-methoxypiperidin-1-yl)pyrimidin-4-yl)amino)pyrido[3,4-d]pyridazin-1-yl)pyrrolidin-1-yl)prop-2-en-1-one,
1-(2-(4-(3-(4,4-dioxido-1,4-oxathian-3-yl)azetidin-1-yl)-7-((2-((3S,4R)-3-fluoro-4-methoxypiperidin-1-yl)pyrimidin-4-yl)amino)pyrido[3,4-d]pyridazin-1-yl)pyrrolidin-1-yl)prop-2-en-1-one,
1-(2-(7-((2-((3S,4R)-3-fluoro-4-methoxypiperidin-1-yl)pyrimidin-4-yl)amino)-4-(3-(4-hydroxy-4-methyl-1,1-dioxidotetrahydro-2H-thiopyran-2-yl)azetidin-1-yl)pyrido[3,4-d]pyridazin-1-yl)pyrrolidin-1-yl)prop-2-en-1-one,
1-(2-(4-(3-(1,1-dioxido-4-(tetrahydro-2H-pyran-4-yl)thiomorpholin-2-yl)azetidin-1-yl)-7-((2-((3S,4R)-3-fluoro-4-methoxypiperidin-1-yl)pyrimidin-4-yl)amino)pyrido[3,4-d]pyridazin-1-yl)pyrrolidin-1-yl)prop-2-en-1-one,
1-(2-(4-(3-(1,1-dioxido-4-(2-(trifluoromethyl)pyridin-4-yl)thiomorpholin-2-yl)azetidin-1-yl)-7-((2-((3S,4R)-3-fluoro-4-methoxypiperidin-1-yl)pyrimidin-4-yl)amino)pyrido[3,4-d]pyridazin-1-yl)pyrrolidin-1-yl)prop-2-en-1-one,
1-(2-(7-((2-((3S,4R)-3-fluoro-4-methoxypiperidin-1-yl)pyrimidin-4-yl)amino)-4-(3-(6-methyl-1,1-dioxidotetrahydro-2H-thiopyran-2-yl)azetidin-1-yl)pyrido[3,4-d]pyridazin-1-yl)pyrrolidin-1-yl)prop-2-en-1-one,
1-(2-(4-(3-(6,6-dimethyl-1,1-dioxidotetrahydro-2H-thiopyran-2-yl)azetidin-1-yl)-7-((2-((3S,4R)-3-fluoro-4-methoxypiperidin-1-yl)pyrimidin-4-yl)amino)pyrido[3,4-d]pyridazin-1-yl)pyrrolidin-1-yl)prop-2-en-1-one,
5-(2-(1-(1-(1-acryloylpyrrolidin-2-yl)-7-((2-((3S,4R)-3-fluoro-4-methoxypiperidin-1-yl)pyrimidin-4-yl)amino)pyrido[3,4-d]pyridazin-4-yl)azetidin-3-yl)-1,1-dioxidothiomorpholino)-2-(2,6-dioxopiperidin-3-yl)isoindoline-1,3-dione,

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. For example:
Deuterium-enriched compounds: deuterium (D or ²H) is a stable, non-radioactive isotope of hydrogen and has an atomic weight of 2.0144. Hydrogen naturally occurs as a mixture of the isotopes ^{x}H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-di ethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 March; 3(3):233-44]. Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The invention encompasses pharmaceutical compositions comprising the compound of the present invention and pharmaceutical excipients, as well as other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-α-tocopheryl PEG 1000 succinate (20-50%), using complexation such as HPβCD and SBEβCD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “Acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl.

The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

Spirocycloalkyl refers to a compound comprising two saturated cyclic alkyl rings sharing only one common atom (also known as a spiro atom), with no heteroatom and no unsaturated bonds on any of the rings. In one embodiment, the spiroalkyl is bicyclic. In another embodiment, the spiroalikyl has more than two cycles. In certain embodiments, the spiroalkyl compound is a polyspiro compound connected by two or more spiroatoms making up three or more rings. In certain embodiments, one of the rings of the bicyclic spiroalkyl has 3, 4, 5, 6, 7, or 8 atoms, including the common spito atom. In one embodiment, the spiroalkyl is a 5 to 20 membered, 5 to 14 membered, or 5 to 10 membered polycyclic spiroalkyl group. Representative examples of spiroalkyl include, but are not limited to the following groups:

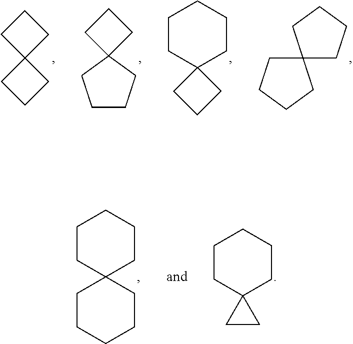

The term “fused-carbocyclic” refers to a polycyclic cyclyl group, wherein each ring in the group shares an adjacent pair of carbon atoms with another ring in the group, wherein one or more rings can contain one or more double bonds. In certain embodiments, the fused heterocyclyl is bicyclic. In certain embodiments, the fused-carbocyclic contains more than two rings, at least two of which share an adjacent pair of atoms. In one embodiment, the fused-carbocyclic is a 5 to 20 membered, 5 to 16 membered, or 5 to 10 membered polycyclic cyclyl group. Representative examples of fused-carbocyclic include, but are not limited to the following groups:

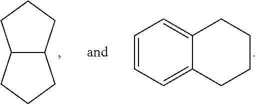 [SMILES: C1CC2CCCC2C1 ; c1ccc2c(c1)CCCC2]

The term “bridged-carbocyclic” refers to a group having at least two rings sharing three or more common ring atoms, separating the two bridgehead atoms by a bridge containing at least one atom. The bridgehead atoms are the atoms from which three bonds radiate and where the rings meet. The rings of the bridged carbocyclyl can have one or more double bonds. In one embodiment, the bridged carbocyclyl is bicyclic. In one embodiment, the bridged carbocyclyl is a 5 to 20 membered, 5 to 16 membered, or 5 to 10 membered polycyclic carbocyclyl group. Representative examples of bridged carbocyclyl include, but are not limited to the following groups:

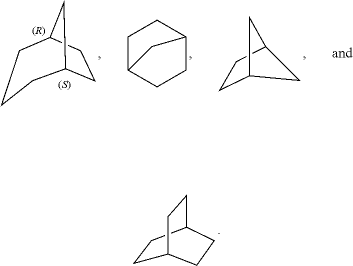

The term “heterocycloalkyl” refers to a nonaromatic 5-8 membered monocyclic, 8-12 membered bicyclic, 11-14 membered tricyclic, or 14-20 membered tetracyclic ring system having one or more heteroatoms (such as O, N, S, P, or Se). Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic 5-8 membered monocyclic, 8-12 membered bicyclic, 11-14 membered tricyclic, or 14-20 membered tetracyclic ring system having one or more heteroatoms (such as O, N, S, P, or Se) and one or more double bonds.

Spiroheterocyclyl refers to a compound comprising two non-saturated rings sharing only one common atom (also known as a spiro atom), with at least one heteroatom on one of the two rings, such as a polycyclic heterocyclyl group with rings connected through one common carbon atom. The common atom can be carbon (C), silicon, or nitrogen (such as a positively charged quaternary nitrogen atom). The heteroatoms can comprise nitrogen, quaternary nitrogen, oxidized nitrogen (e.g., NO), oxygen, silicon, and sulfur, including sulfoxide and sulfone, and the remaining ring atoms are C. In addition, one or more of the rings may contain one or more double bonds. In one embodiment, the spiro heterocyclyl is bicyclic, with heteroatom(s) on either one or both cycles. In certain embodiments, one of the rings of the bicyclic spiro heterocyclyl has 3, 4, 5, 6, 7, or 8 atoms, including the common spito atom. In certain embodiments, the spiro heterocyclic compound is a polyspiro compound connected by two or more spiroatoms making up three or more rings. In one embodiment, the spiro heterocyclyl is a 5 to 20 membered, 5 to 14 membered, or 5 to 10 membered polycyclic heterocyclyl group. Representative examples of spiro heterocyclyl include, but are not limited to the following groups:

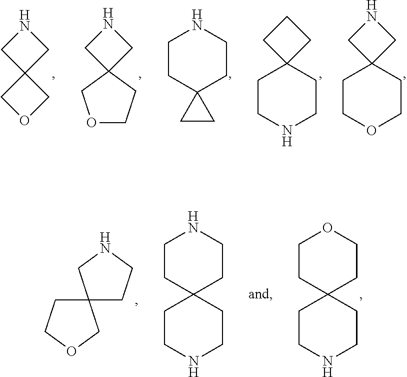

Fused heterocyclyl refers to a polycyclic heterocyclyl group, wherein each ring in the group shares an adjacent pair of atoms (such as carbon atoms) with another ring in the group, wherein one or more rings can contain one or more double bonds, and wherein said rings have one or more heteroatoms, which can be nitrogen, quaternary nitrogen, oxidized nitrogen (e.g., NO), oxygen, and sulfur, including sulfoxide and sulfone, and the remaining ring atoms are C. In certain embodiments, the fused heterocyclyl is bicyclic. In certain embodiments, the fused heterocyclyl contains more than two rings, at least two of which share an adjacent pair of atoms. In one embodiment, the fused heterocyclyl is a 5 to 20 membered, 5 to 16 membered, or 5 to 10 membered polycyclic heterocyclyl group. Representative examples of fused heterocyclyl include, but are not limited to the following groups:

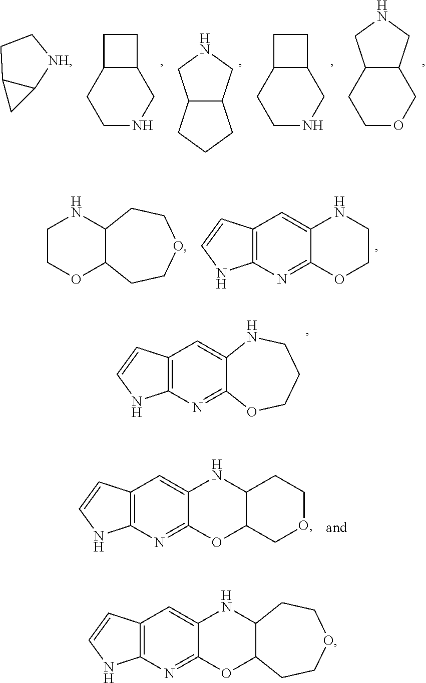

Bridged heterocyclyl refers to a compound having at least two rings sharing three or more common ring atoms, separating the two bridgehead atoms by a bridge containing at least one atom, wherein at least one ring atom is a heteroatom. The bridgehead atoms are the atoms from which three bonds radiate and where the rings meet. The rings of the bridged heterocyclyl can have one or more double bonds, and the ring heteroatom(s) can be nitrogen, quaternary nitrogen, oxidized nitrogen (e.g., NO), oxygen, and sulfur, including sulfoxide and sulfone as ring atoms, while the remaining ring atoms are C. In one embodiment, the bridged heterocyclyl is bicyclic. In one embodiment, the bridged heterocyclyl is a 5 to 20 membered, 5 to 16 membered, or 5 to 10 membered polycyclic heterocyclyl group. Representative examples of bridged heterocyclyl include, but are not limited to the following groups:

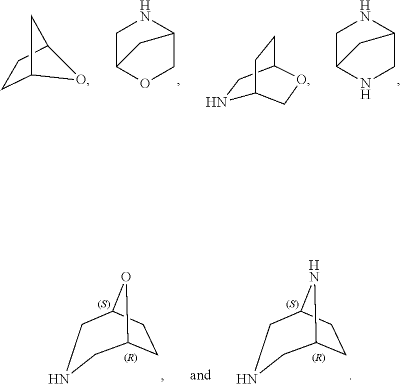

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl.

The term “heteroaryl” refers to an aromatic 5-8 membered monocyclic, 8-12 membered bicyclic, or 11-14 membered tricyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Alkyl, alkenyl, alkynyl, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, mentioned above include both substituted and unsubstituted moieties. Possible substituents on alkylamino, cycloalkyl, heterocycloalkyl, cycloalkenyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thioxo (S═), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmaceutically acceptable carrier” means a non-toxic solvent, dispersant, excipient, adjuvant, or other material which is mixed with the compounds of the present invention in order to form a pharmaceutical composition, i.e., a dose form capable of administration to the patient. Examples of pharmaceutically acceptable carrier includes suitable polyethylene glycol (e.g., PEG400), surfactant (e.g., Cremophor), or cyclopolysaccharide (e.g., hydroxypropyl-β-cyclodextrin or sulfobutyl ether β-cyclodextrins), polymer, liposome, micelle, nanosphere, etc.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors(Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMK1a, CAMK1b, CAMK1d, CAMK1g, CAMKIla, CAMKIIb, CAMKIId, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin 11, CHK1, CHK2, CKlal, CK1d, CKlepsilon, CK1g1, CK1g2, CK1g3, CK2a, CK2a2, c-KIT, CLK1, CLK2, CLK3, CLK4, c-MER, c-MET, COT1/MAP3K8, CSK, c-SRC, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRKIA, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1/VEGFR1, FLT3, FLT4/VEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDRNEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPKS/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MY03b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6KB1, p70S6Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), PI3Kg(p120g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TA01, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYR03/SKY, ULK1, ULK2, ULK3, VRK1, VRK2, WEE1, WNK1, WNK2, WNK3, YES/YES1, ZAK/MLTK, ZAP70, ZIPK/DAPK3, KI NASE, MUTANTS, ABL1 (E255K), ABL1 (F3171), ABL1 (G250E), ABL1(H396P), ABL1(M351T), ABL1(Q252H), ABL1(T3151), ABL1(Y253F), ALK (C1156Y), ALK(L1196M), ALK (F1174L), ALK (R1275Q), BRAF(V599E), BTK(E41K), CHK2(1157T), c-Kit(A829P), c-KIT(D816H), c-KIT(D816V), c-Kit(D820E), c-Kit(N822K), C-Kit (T6701), c-Kit(V559D), c-Kit(V559D/V654A), c-Kit(V559D/T6701), C-Kit (V560G), c-KIT(V654A), C-MET(D1228H), C-MET(D1228N), C-MET(F12001), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (12020T), LRRK2 (R1441C), p38a(T106M), PDGFRa(D842V), PDGFRa(T6741), PDGFRa(V561D), RET(E762Q), RET(G691 S), RET(M918T), RET(R749T), RET(R813Q), RET(V804L), RET(V804M), RET(Y791F), TI F2(R849W), TI F2(Y897S), and TI F2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g. HSP90), poly-ADP (adenosine diphosphate)-ribose polymerase (PARR), hypoxia-inducible factors(HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc).

In another aspect of the invention, the compounds of the invention are administered in combination with one or more of other anti-cancer agents that include, but are not limited to, gene therapy, RNAi cancer therapy, chemoprotective agents (e.g., amfostine, mesna, and dexrazoxane), drug-antibody conjugate (e.g brentuximab vedotin, ibritumomab tioxetan), cancer immunotherapy such as Interleukin-2, cancer vaccines (e.g., sipuleucel-T) or monoclonal antibodies (e.g., Bevacizumab, Alemtuzumab, Rituximab, Trastuzumab, etc).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, antimetabolites, topoisomerase inhibitors, anti-microtubule agents, kinase inhibitors, epigenetic agents, HSP90 inhibitors, PARR inhibitors, BCL-2 inhibitor, drug-antibody conjugate, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, radiation therapy, or surgery.

In certain embodiments, the compounds of the invention are administered in combination with one or more anti-inflammatory agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates. The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-05 monoclonal antibody (such as eculizumab or pexelizumab), a TNF antagonist, such as entanercept, or infliximab, which is an anti-TNF alpha monoclonal antibody.

In certain embodiments, the compounds of the invention are administered in combination with one or more immunosuppressant agents.

In some embodiments, the immunosuppressant agent is glucocorticoid, methotrexate, cyclophosphamide, azathioprine, mercaptopurine, leflunomide, cyclosporine, tacrolimus, and mycophenolate mofetil, dactinomycin, anthracyclines, mitomycin C, bleomycin, or mithramycin, or fingolimod.

The invention further provides methods for the prevention or treatment of a neoplastic disease, autoimmune and/or inflammatory disease. In one embodiment, the invention relates to a method of treating a neoplastic disease, autoimmune and/or inflammatory disease in a subject in need of treatment comprising administering to said subject a therapeutically effective amount of a compound of the invention. In one embodiment, the invention further provides for the use of a compound of the invention in the manufacture of a medicament for halting or decreasing a neoplastic disease, autoimmune and/or inflammatory disease.

In one embodiment, the neoplastic disease is a B-cell malignancy includes but not limited to B-cell lymphoma, lymphoma (including Hodgkin's lymphoma and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia.

The autoimmune and/or inflammatory diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barré syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of reaction schemes. Necessary starting materials may be obtained by standard procedures of organic chemistry. The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

An approach to synthesize compounds of

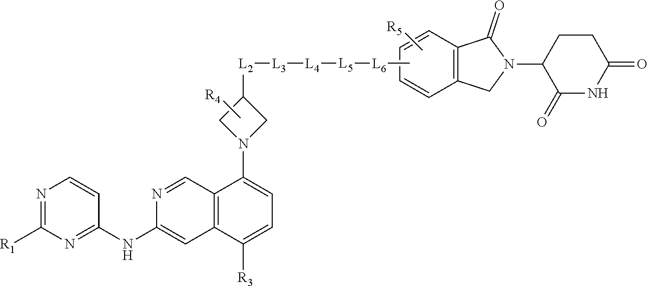

is described in Scheme 1. R₁, R₃, R₄, R₅, L₂, L₃, L₄, L₅, L₆, m, n, and i, in general Scheme 1 is the same as those described in the Summary section above.

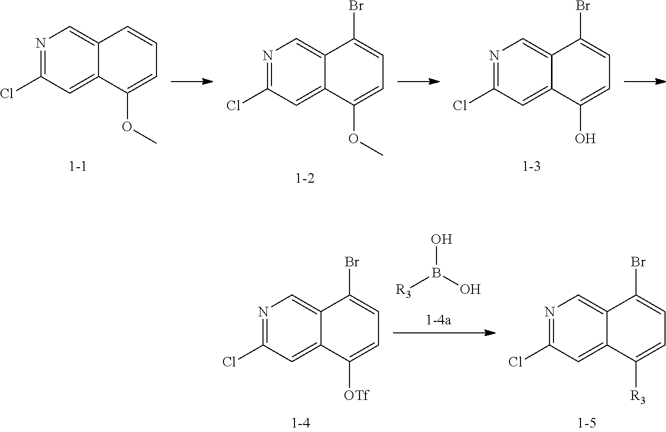

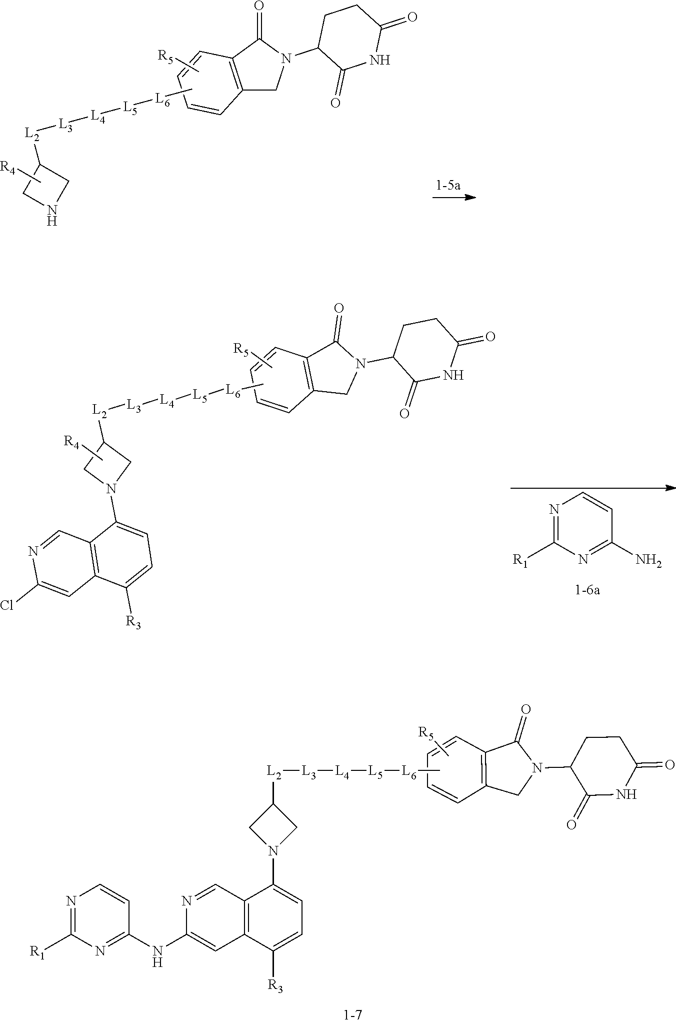

In Scheme 1, the starting material 1-1 can be prepared by conventional procedures using appropriate compounds and reagents. 1-1 can be converted to 1-2 under a conventional condition, and then the intermediate 1-2 is demethylated to give 1-3. After that, 1-3 can be converted to 1-4 readily, 0-triflate group of which can selectively couple with 1-4a to give 1-5. Next, 1-5 undergoes coupling reaction with 1-5a to afford 1-6. Finally, the target compounds I-7 can be obtained by aryl amination reaction of 1-6 with 1-6a.

Also, the target compounds can be synthesized by alternative methods but not limited to the above procedures.

The compounds of

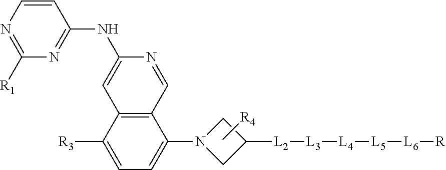

can be made by the method referred to Scheme 1 by using variable starting material, intermediates, and reagents.

The compounds of

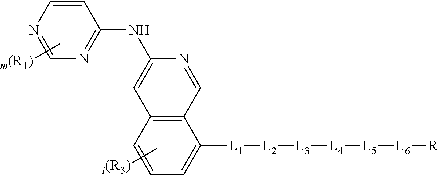

can be made by the method referred to Scheme 1 by using variable starting material, intermediates, and reagents.

The compounds of

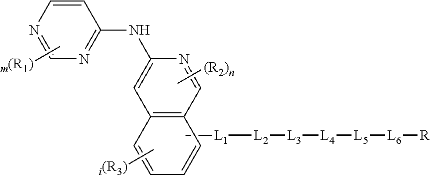

can be made by the method referred to Scheme 1 by using variable starting material, intermediates, and reagents.

### Part II

An approach to synthesize compounds of

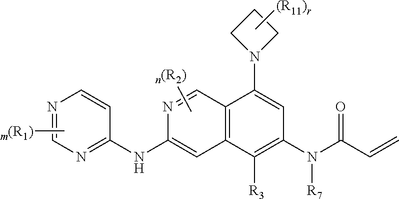

is described in Scheme A1 and Scheme A2. R₁, R₂, R₃, R₇, R₁₁, m, n, and r, in General Scheme A1 and Scheme A2 is the same as

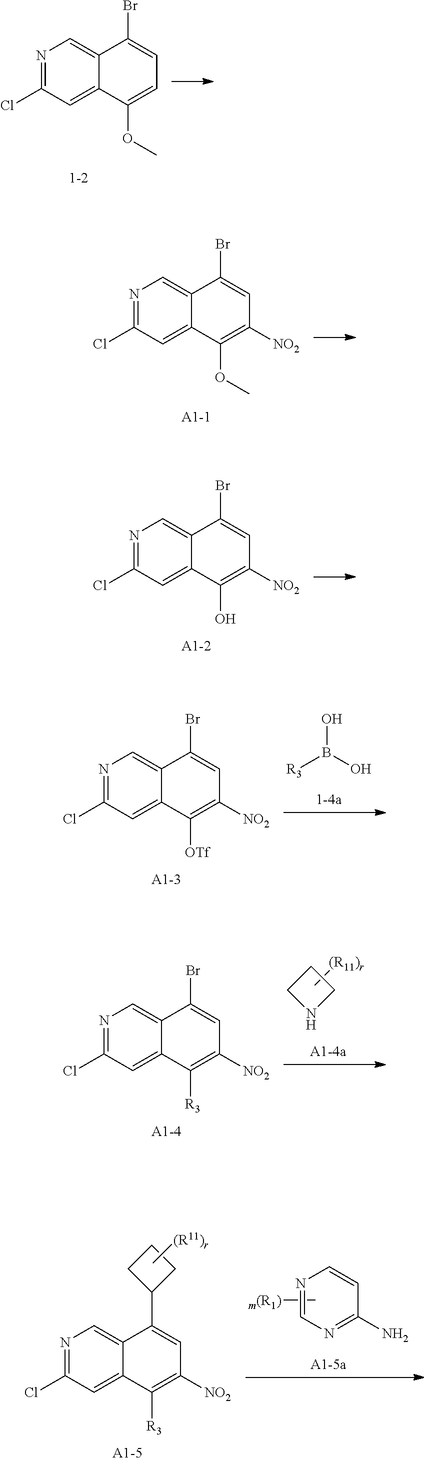

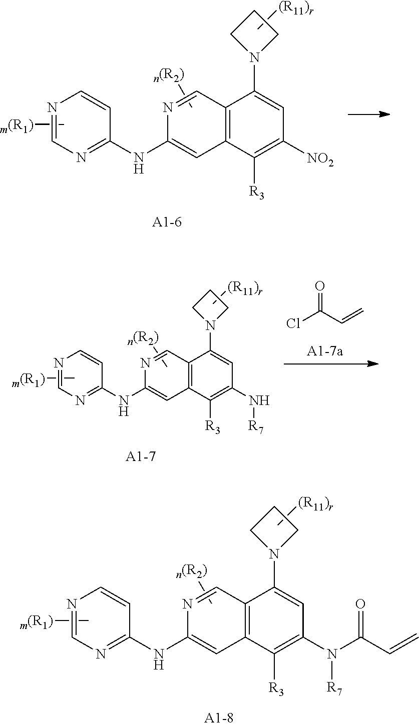

In Scheme A1-1, the starting material 1-2 can be prepared by conventional procedures using appropriate compounds and reagents. The starting material 1-2 is converted to A1-1 under a nitration condition. A1-1 can be demethylated to give A1-2, which is further converted to A1-3 readily. After that, the intermediate A1-3 goes through a Suzuki coupling reaction with 1-4a to yield A1-4, which can subsequently couple with A1-4a to give A1-5. Next, the chloride A1-5 can react with A1-5a to afford A1-6, and then A1-6 is reduced to yield aniline A1-7. Finally, the condensation of A1-7 with A1-7a can afford the target compounds A1-8.

Also, the target compounds can be synthesized by alternative methods but not limited to the above procedures.

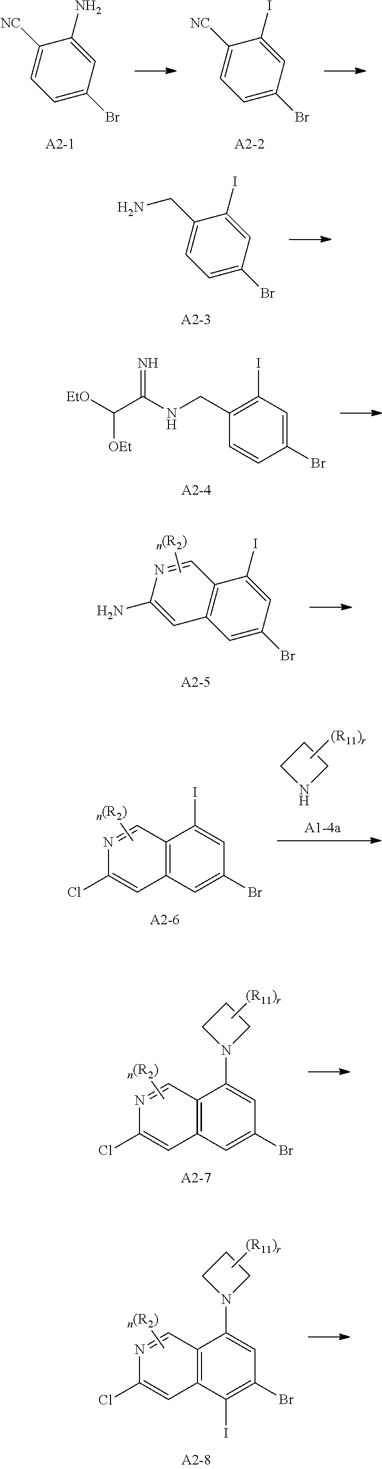

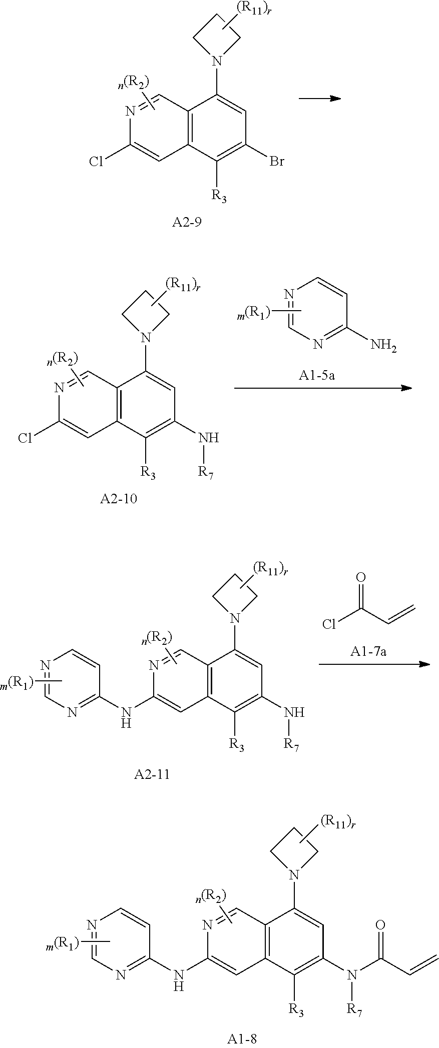

In Scheme A-2, the starting material A2-1 can be prepared by conventional procedures using appropriate compounds and reagents. The starting material A2-1 is converted to A2-2 via Sandmeyer reaction. The intermediate A2-2 can be reduced to give A2-3, which can be converted to A2-4 readily. After that, the intermediate A2-4 goes through an intramolecular cyclization to yield A2-5, which can subsequently be converted to A2-6. Next, the intermediate A2-6 can react with A1-4a to afford A2-7, which goes through an iodination reaction to afford A2-8. The intermediate A2-8 is converted to A2-11 via a sequence of three-step coupling reactions. Finally, the condensation of A2-11 with A1-7a can afford the target compounds A1-8.

Also, the target compounds can be synthesized by alternative methods but not limited to the above procedures.

An approach to synthesize compounds of

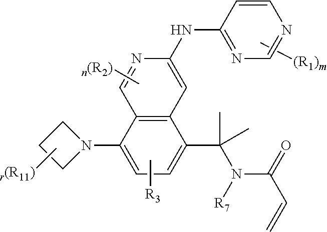

is described in Scheme B1 and Scheme B2. R₁, R₂, R₃, R₇, R₁₁, m, n, and r, in General Scheme B1 and Scheme B2 is the same as those described in the Summary section above.

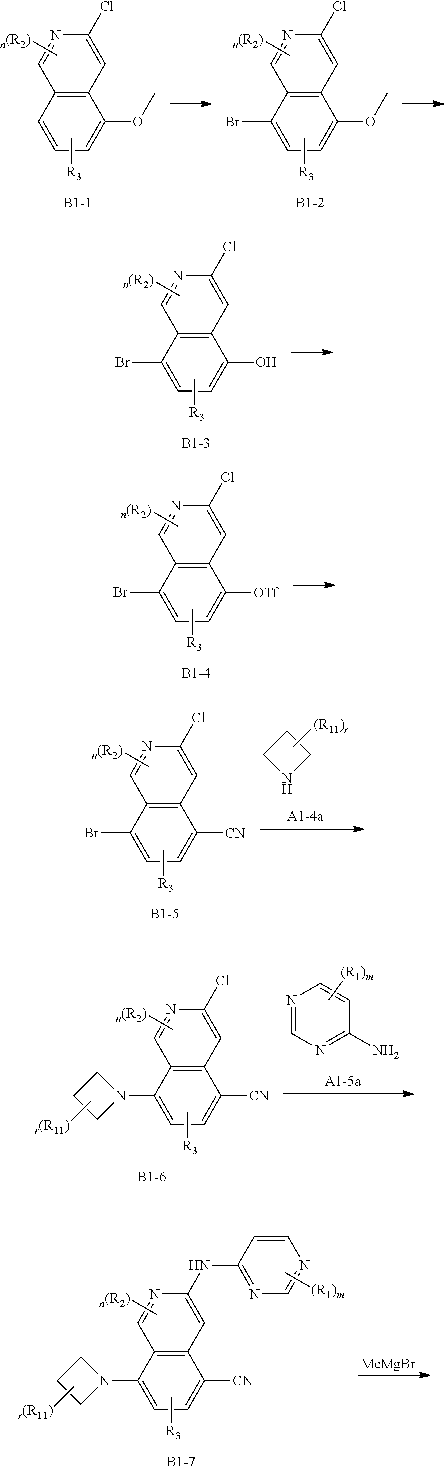

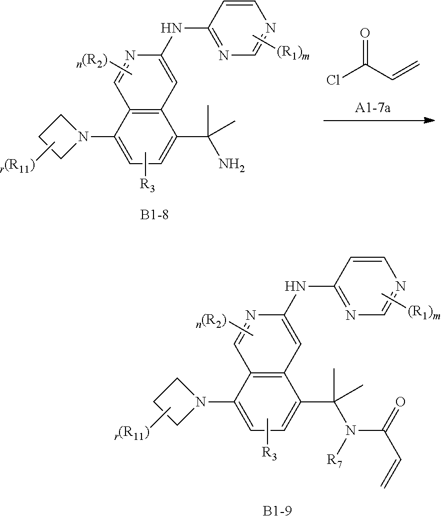

In Scheme B1, the starting material B1-1 can be prepared by conventional procedures using appropriate compounds and reagents. B1-1 can be converted to B1-2 under a conventional condition, and then B1-2 can undergo a demethylation reaction to give B1-3. After that, B1-3 can be converted to B1-4 readily, which can go through a coupling reaction to give the cyanide B1-5. Next, B1-5 can couple with A1-4a to afford B1-6, which further reacts with A1-5a to give the intermediate B1-7. The intermediate B1-7 can undergo a double Grignard reaction to give B1-8. Finally, the condensation of B1-8 with A1-7a can afford the target compounds B1-9.

Also, the target compounds can be synthesized by alternative methods but not limited to the above procedures.

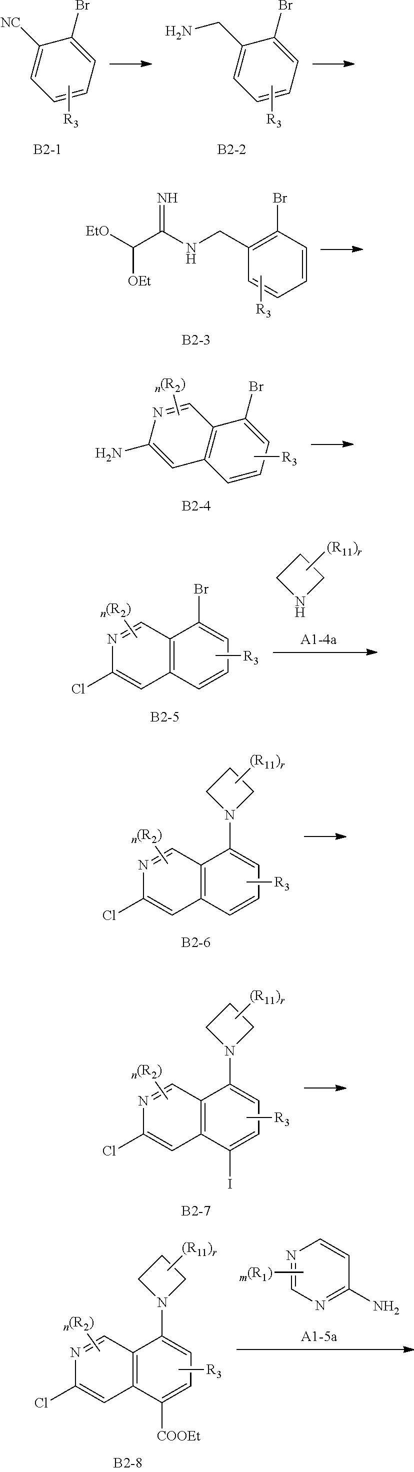

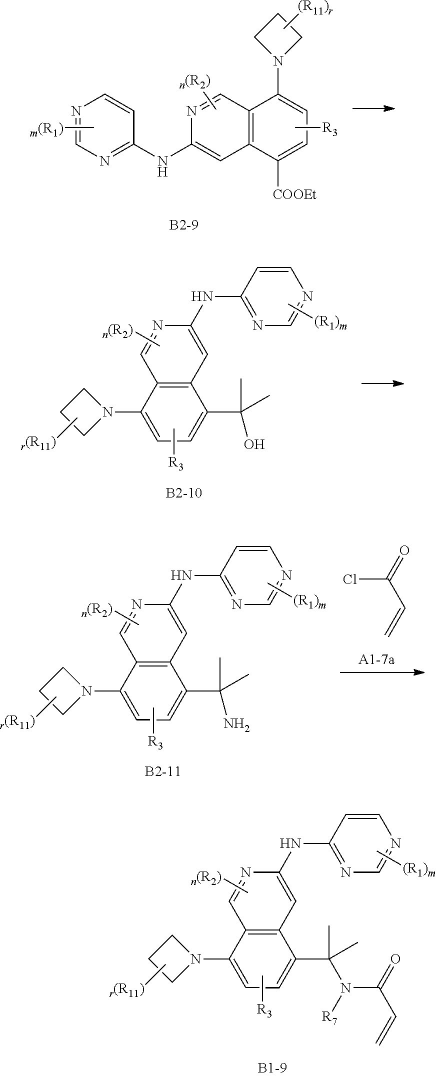

In Scheme B2, the starting material B2-1 can be prepared by conventional procedures using appropriate compounds and reagents. B2-1 is reduced to B2-2 under a conventional condition, which can be converted to B2-3. After that, B2-3 goes through an intramolecular cyclization to give B2-4, which can be converted to B2-5 via Sandmeyer reaction. Next, B2-5 can couple with A1-4a to afford B2-6, which is converted to B2-7 through an iodization reaction. B2-7 can undergo a Pd-catalyzed carbonylation to give B2-8, which can be converted to B2-9 readily. Finally, the intermediate B2-9 is converted to B2-11 via a literate-known condition, and then B2-11 react with A1-7a can afford the target compounds B1-9.

Also, the target compounds can be synthesized by alternative methods but not limited to the above procedures.

An approach to synthesize compounds of

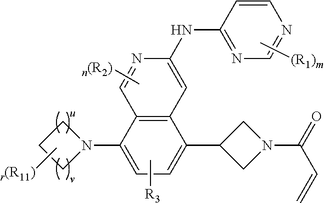

is described in Scheme C1 and Scheme C2.

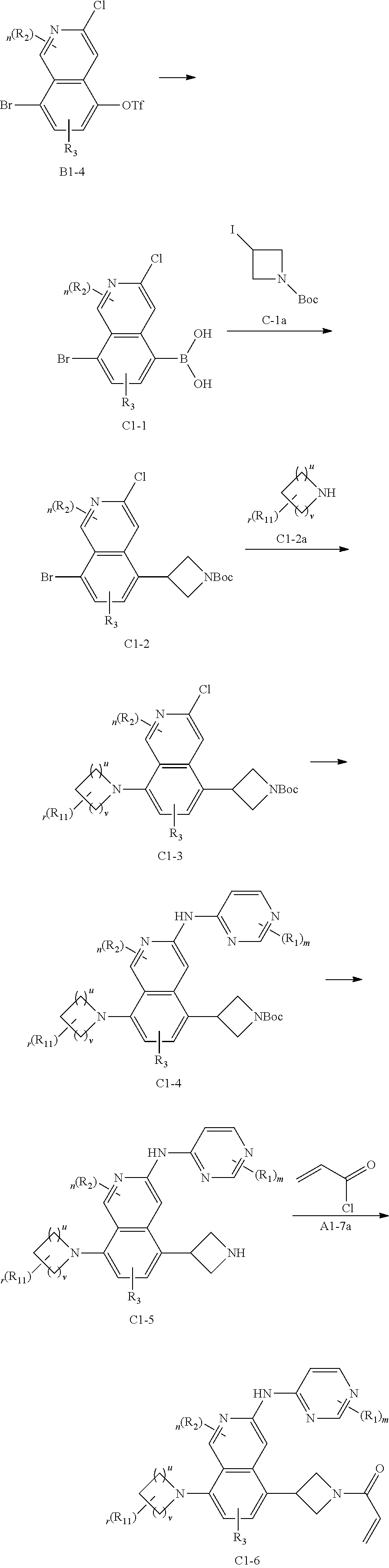

In Scheme C1, the starting material B1-4 can be prepared by conventional procedures using appropriate compounds and reagents. The starting material B1-4 can be converted to boronic acid C1-1 readily, which undergoes literate-known coupling reactions to generate C1-2. After that, the intermediate C1-2 undergoes a two-step sequence of Buchwald coupling reaction to yield C1-4. Finally, the intermediate C1-4 is deprotected to give C1-5, which is further treated with A1-7a to afford the target compounds C1-6.

Also, the target compounds can be synthesized by alternative methods but not limited to the above procedures.

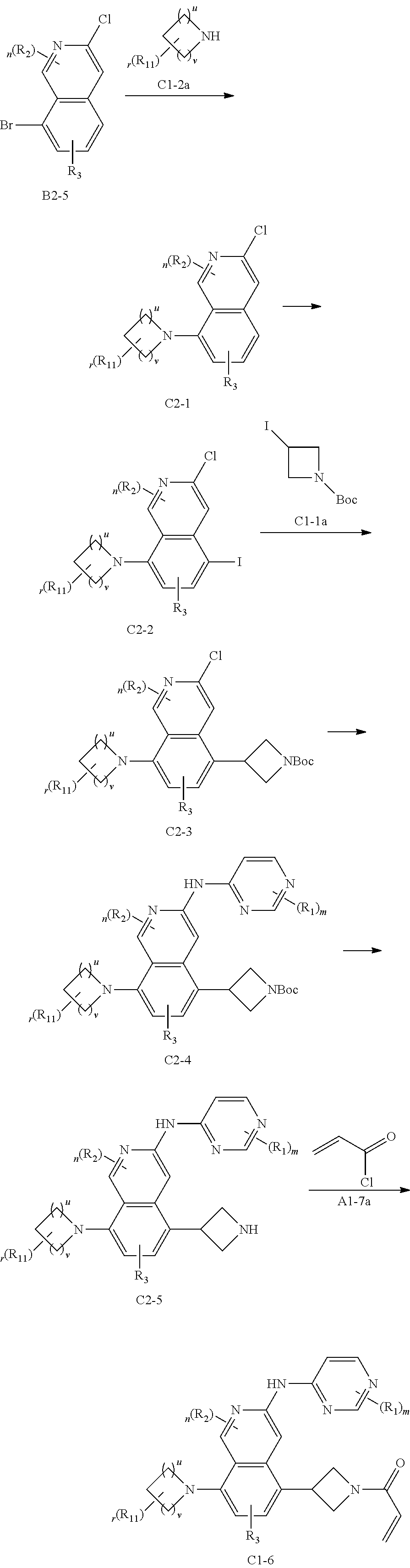

In Scheme C2, the starting material B2-5 can be prepared by conventional procedures using appropriate compounds and reagents. The starting material B2-5 can be converted to C2-1 readily, which undergoes an iodization reaction to generate C2-2. After that, the intermediate C2-2 can react with C1-1a to yield C2-3, which undergoes a Buchwald coupling reaction to afford C2-4. Finally, the intermediate C2-4 is deprotected to give C2-5, which is further treated with A1-7a to afford the target compounds C1-6.

Also, the target compounds can be synthesized by alternative methods but not limited to the above procedures.

An approach to synthesize compounds of

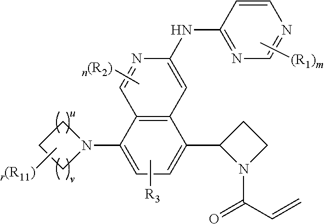

is described in Scheme D1 and Scheme D2.

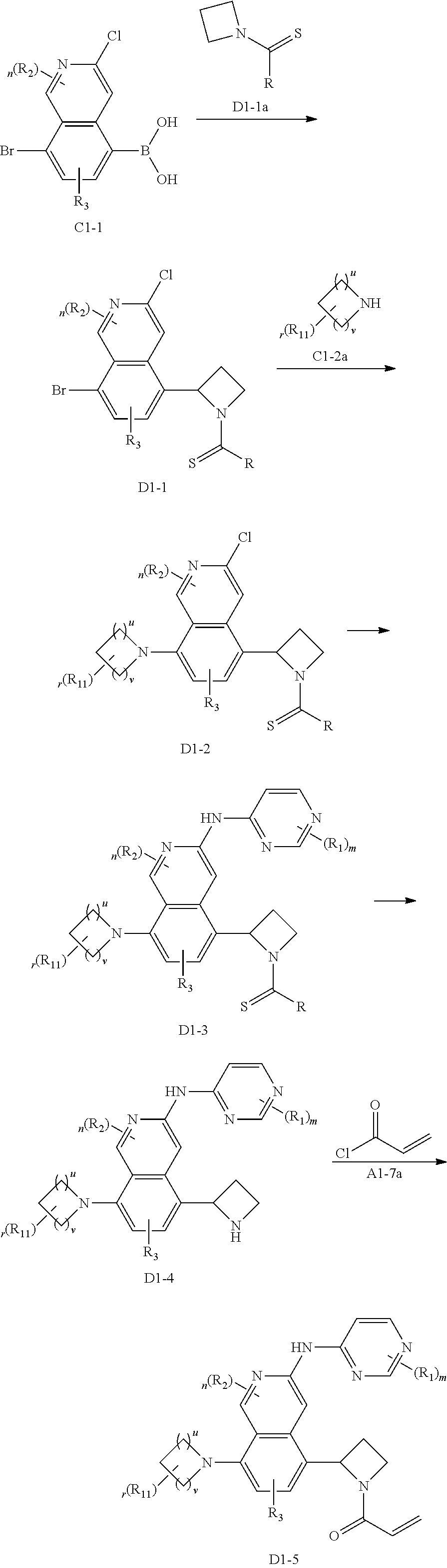

In Scheme D1, the starting material C1-1 can be prepared by conventional procedures using appropriate compounds and reagents. The starting material C1-1 can be converted to D1-1 via literate-known reactions. After that, the intermediate D1-1 undergoes a two-step sequence of Buchwald coupling reaction to yield D1-3. Finally, the intermediate D1-3 is deprotected to give D1-4, which is further treated with A1-7a to afford the target compounds D1-5.

Also, the target compounds can be synthesized by alternative methods but not limited to the above procedures.

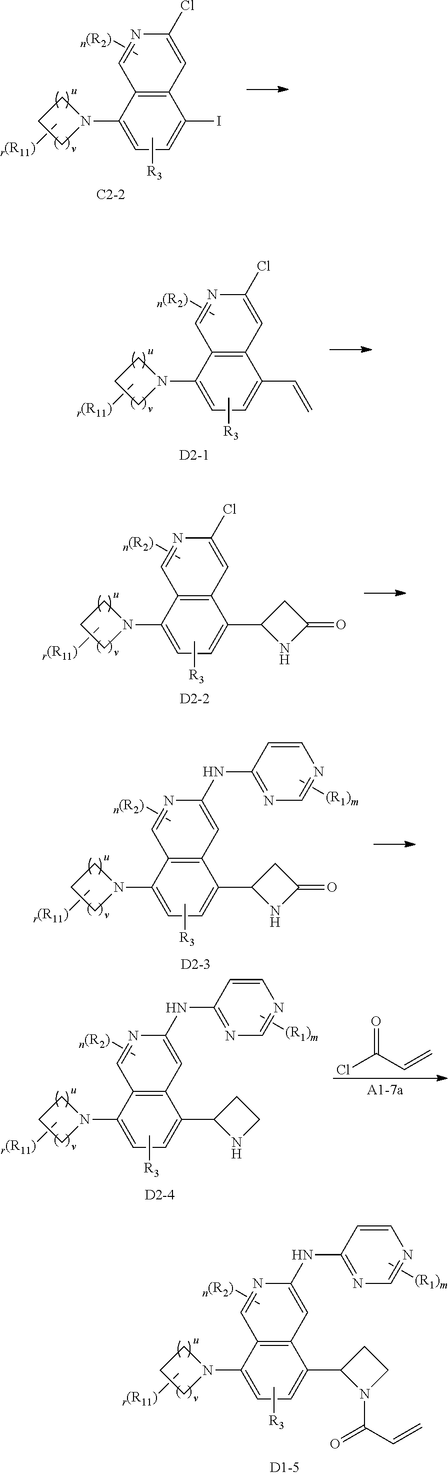

In Scheme D2, the starting material C₂₋₂ can be prepared by conventional procedures using appropriate compounds and reagents. The starting material C₂₋₂ can be converted to D2-1 via Suzuki coupling. After that, the intermediate D2-1 undergoes an intramolecular cyclization to yield D2-2, which undergoes Buchwald coupling reaction to yield D2-3. Finally, the intermediate D2-3 is reduced to give D2-4, which is further treated with A1-7a to afford the target compounds D1-5.

Also, the target compounds can be synthesized by alternative methods but not limited to the above procedures.

An approach to synthesize compounds of

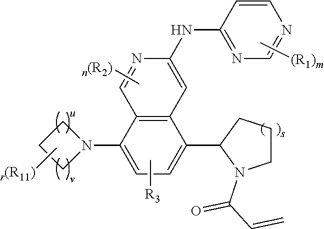

is described in Scheme E1 and Scheme E2.

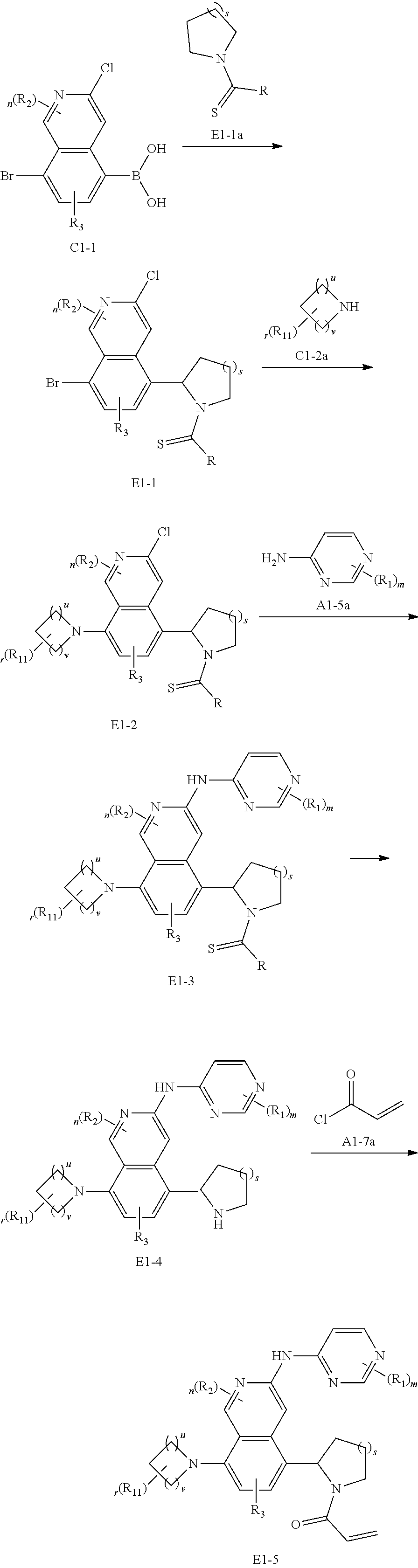

In Scheme E1, the starting material C1-1 can be prepared by conventional procedures using appropriate compounds and reagents. The starting material C1-1 can be converted to E1-1 via literate-known condition. After that, the intermediate E1-1 undergoes a two-step sequence of Buchwald coupling reaction to yield E1-3. Finally, the intermediate E1-3 is deprotected to give E1-4, which is further treated with A1-7a to afford the target compounds E1-5.

Also, the target compounds can be synthesized by alternative methods but not limited to the above procedures.

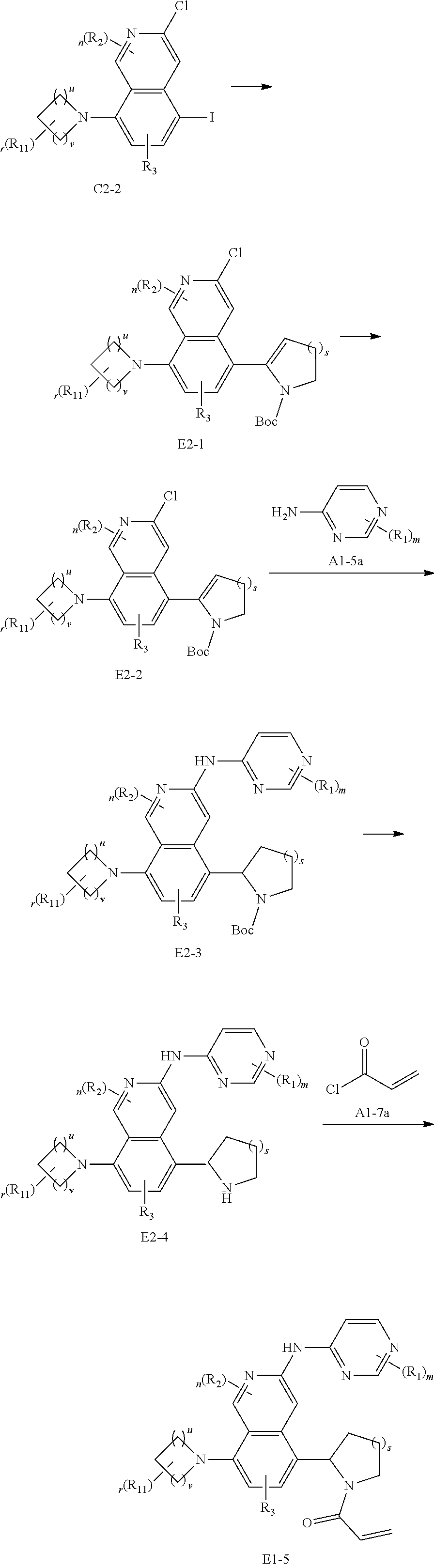

In Scheme E2, the starting material C2-2 can be prepared by conventional procedures using appropriate compounds and reagents. The starting material C2-2 can be converted to E2-1 via literate-known condition. After that, the intermediate E2-1 can be hydrogenated to give E2-2, which undergoes Buchwald coupling reaction to yield E2-3. Finally, the intermediate E2-3 is deprotected to give E2-4, which is further treated with A1-7a to afford the target compounds E1-5.

Also, the target compounds can be synthesized by alternative methods but not limited to the above procedures.

An approach to synthesize compounds of

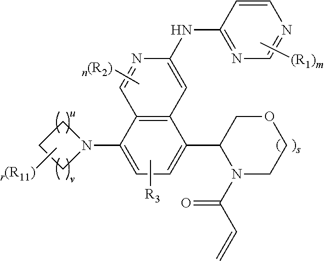

is described in Scheme F1 and Scheme F2.

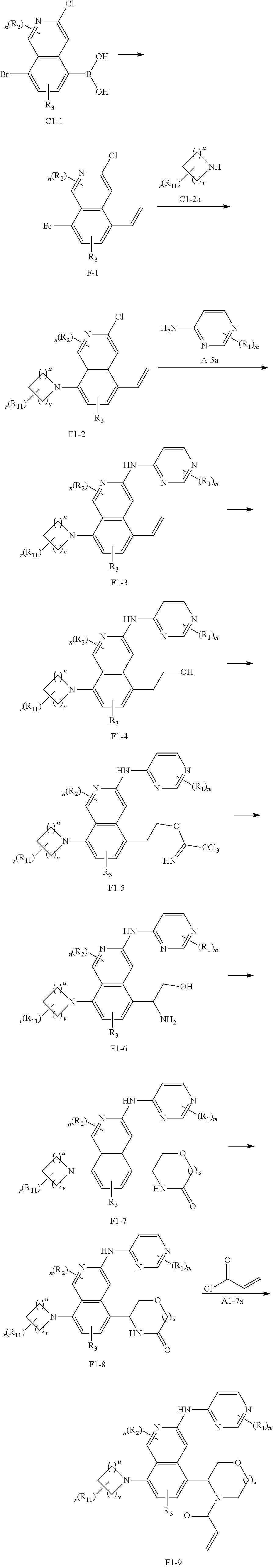

In Scheme F1, the starting material C1-1 can be prepared by conventional procedures using appropriate compounds and reagents. The starting material C1-1 can be converted to F1-1 via a Suzuki coupling reaction. After that, the intermediate F1-1 undergoes a two-step sequence of Buchwald coupling reaction to yield F1-3. The intermediate F1-3 undergoes the Brown hydroboration reaction to generate F1-4, which can be converted to F1-5 readily. Next, the intermediate F1-5 is converted to F1-6 via literate-known conditions, which undergoes an intramolecular cyclization reaction to afford F1-7. Finally, the intermediate F1-7 is reduced to yield F1-8, which is further treated with A1-7a to afford the target compounds F1-9.

Also, the target compounds can be synthesized by alternative methods but not limited to the above procedures.

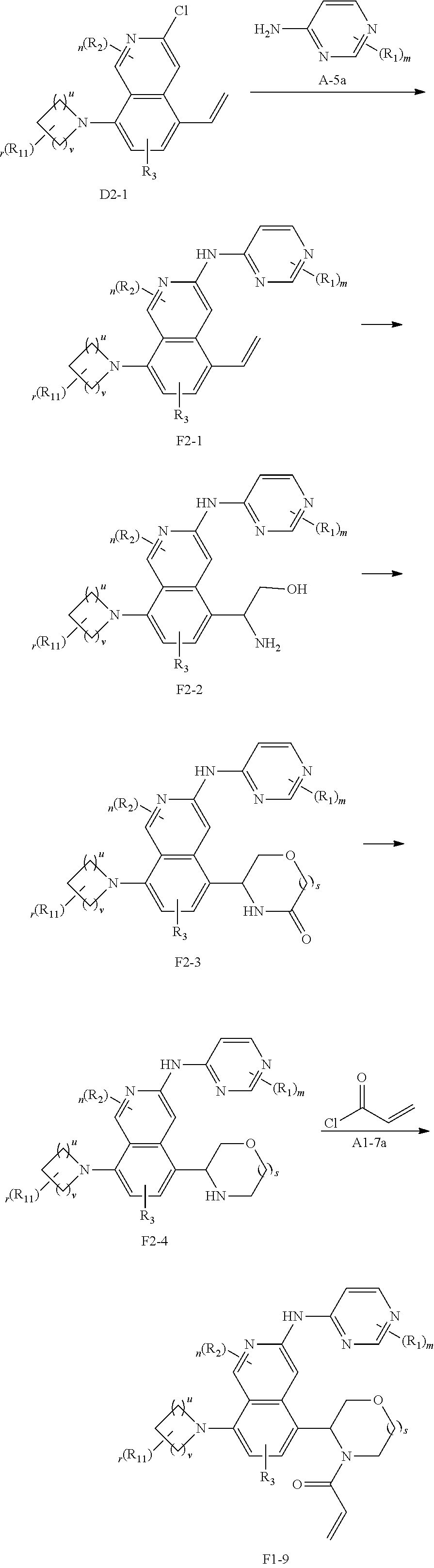

In Scheme F2, the starting material D2-1 can be prepared by conventional procedures using appropriate compounds and reagents. The starting material D2-1 can be converted to F2-1 via a Buchwald coupling reaction. After that, the intermediate F2-1 undergoes Sharpless aminohydroxylation reaction to yield F2-2, which can be converted to F2-3 readily. Finally, the intermediate F2-3 is reduced to yield F2-4, which is further treated with A1-7a to afford the target compounds F1-9.

Also, the target compounds can be synthesized by alternative methods but not limited to the above procedures.

An approach to synthesize compounds of

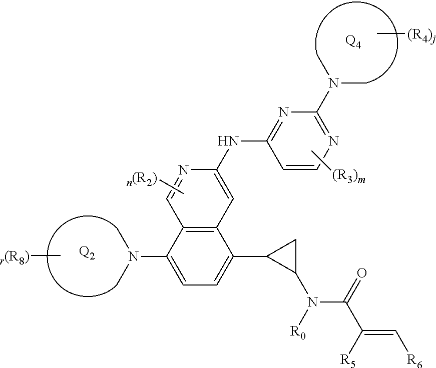

is described in Scheme G.

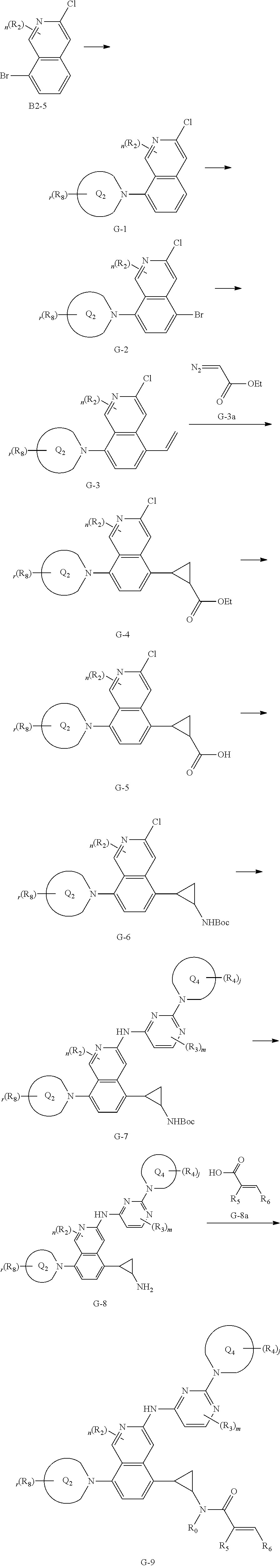

In Scheme G, the starting material B2-5 can be prepared by conventional procedures using appropriate compounds and reagents. The starting material B2-5 can be converted to G-1 via a Buchwald coupling reaction. After that, the intermediate G-1 undergoes bromination reaction to give G-2, which can be converted to G-3 readily. The intermediate G-3 can react with G-3a to afford G-4. Next, G-4 is hydrolyzed to give G-5, which can be converted to G-6 via Curtius rearrangement reaction. The intermediate G-6 can go through a Buchwald coupling reaction to give G-7. Finally, the intermediate G-7 is deprotected to yield G-8, which is further treated with G-8a to afford the target compounds G-9.

Also, the target compounds can be synthesized by alternative methods but not limited to the above procedures.

An approach to synthesize compounds of

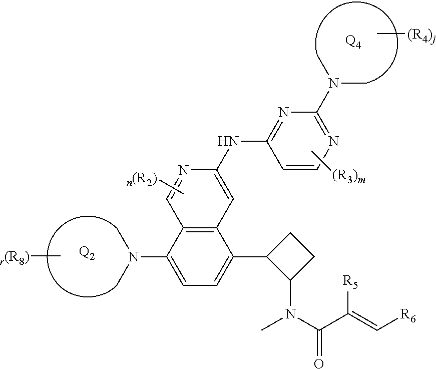

is described in Scheme H.

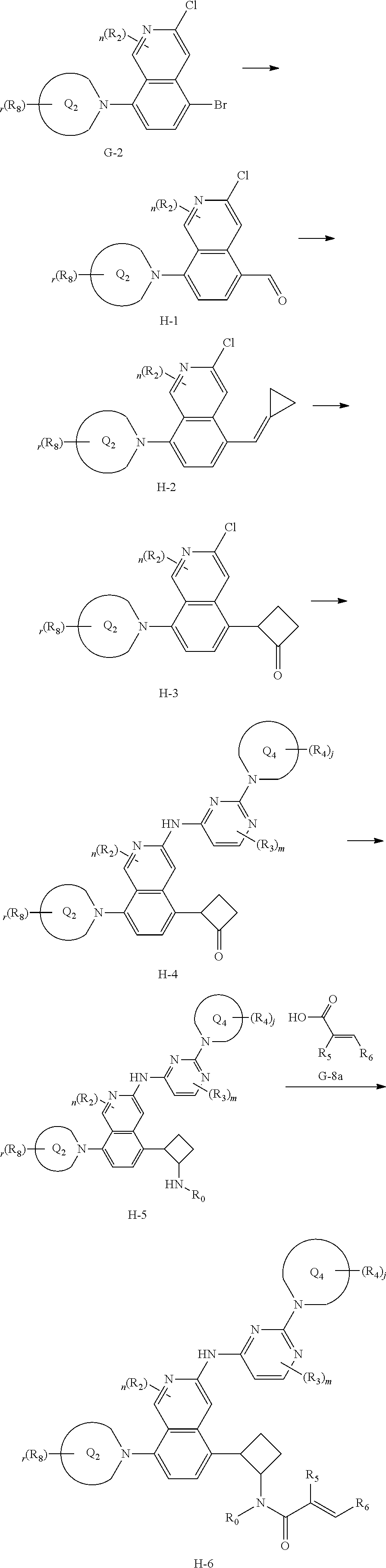

In Scheme H, the starting material G-2 can be prepared by conventional procedures using appropriate compounds and reagents. The starting material G-2 can be converted to H-3 via a literate-known method. After that, the intermediate H3 undergoes Buchwald coupling reaction to yield H-4. Finally, the intermediate H-4 can go through a reductive amination reaction to give H-5, which is further treated with G-8a to afford the target compounds H-6.

Also, the target compounds can be synthesized by alternative methods but not limited to the above procedures.

An approach to synthesize compounds of

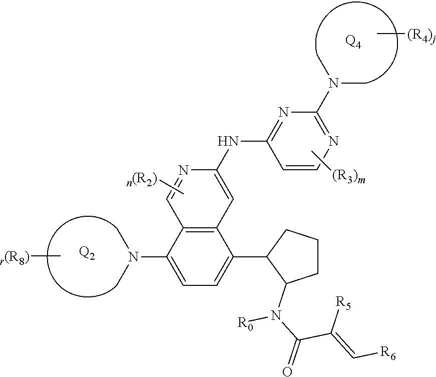

is described in Scheme I.

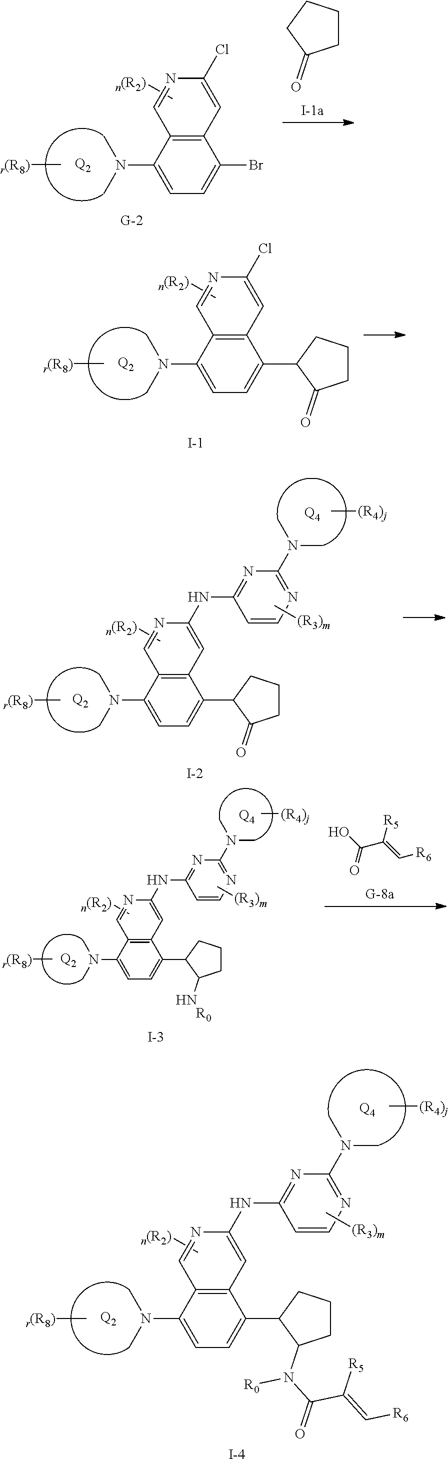

In Scheme I, the starting material G-2 can be prepared by conventional procedures using appropriate compounds and reagents. The starting material G-2 can be converted to I-1 via a literate-known condition. After that, the intermediate I-1 undergoes Buchwald coupling reaction to yield I-2. Finally, the intermediate I-2 can go through a reductive amination reaction to give I-3, which is further treated with G-8a to afford the target compounds I-4.

Also, the target compounds can be synthesized by alternative methods but not limited to the above procedures.

An approach to synthesize compounds of

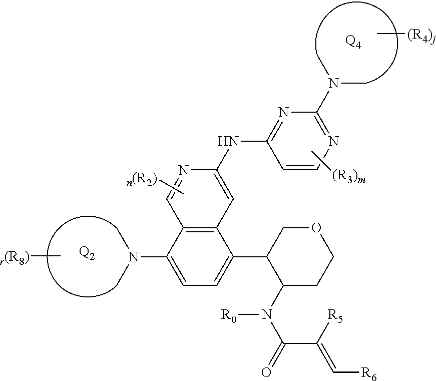

is described in Scheme J.

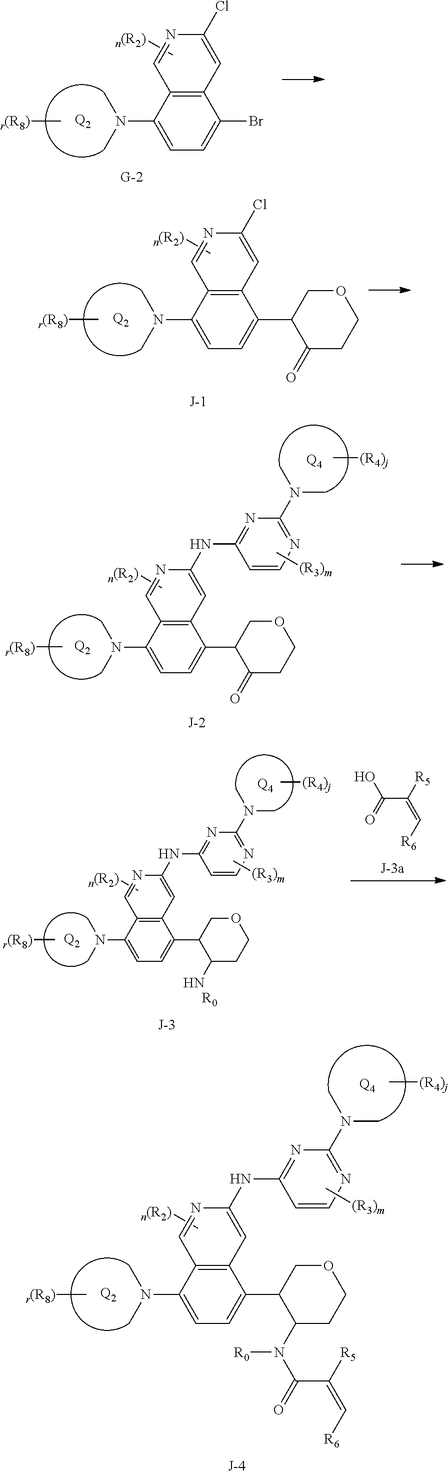

In Scheme J, the starting material G-2 can be prepared by conventional procedures using appropriate compounds and reagents. The starting material G-2 can be converted to J-1 via a literate-known condition. After that, the intermediate J-1 undergoes Buchwald coupling reaction to yield J-2. Finally, the intermediate J-2 can go through a reductive amination reaction to give J-3, which is further treated with J-3a to afford the target compounds J-4.

Also, the target compounds can be synthesized by alternative methods but not limited to the above procedures.

An approach to synthesize compounds of

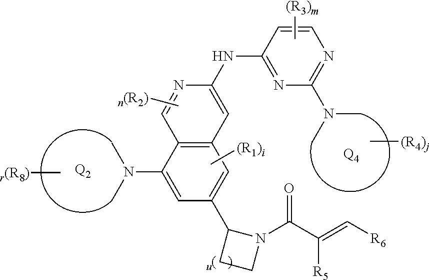

in which u is 1, 2, or 3, is described in Scheme K1, Scheme K2 and Scheme K3.

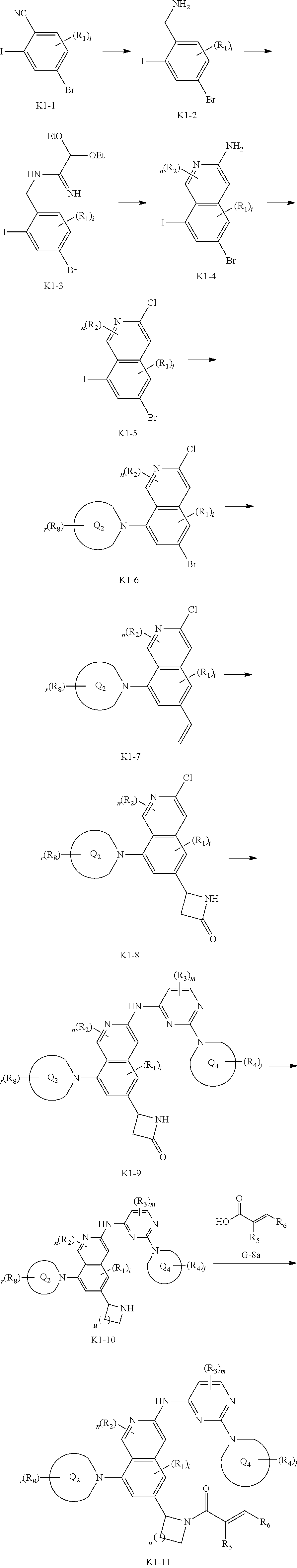

In Scheme K1, the starting material K1-1 can be prepared by conventional procedures using appropriate compounds and reagents. The starting material K1-1 can be converted to K1-5 via a literate-known method. After that, the intermediate K1-5 undergoes Buchwald coupling reaction to yield K1-6, which can be converted to K1-7 readily. Next, the intermediate K1-7 go through an intramolecular cyclization to give K1-8, which undergoes a Buchwald coupling reaction to generate K1-9. Finally, the intermediate K1-9 is reduced to give K1-10, which is further treated with G-8a to afford the target compounds K1-11.

Also, the target compounds can be synthesized by alternative methods but not limited to the above procedures.

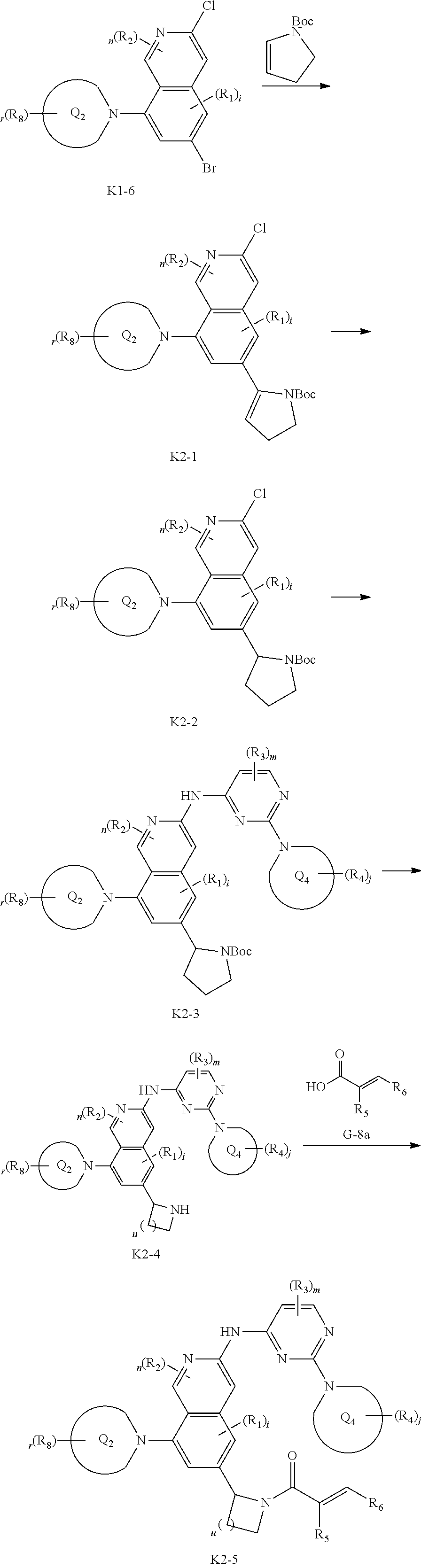

In Scheme K2, the starting material K1-6 can be prepared by conventional procedures using appropriate compounds and reagents. The starting material K1-6 can be converted to K2-1 via Heck coupling reaction. After that, the intermediate K2-1 is hydrogenated to yield K2-2, which undergoes Buchwald coupling reaction to yield K2-3. Finally, the intermediate K2-3 is deprotected to give K2-4, which is further treated with G-8a to afford the target compounds K2-5.

Also, the target compounds can be synthesized by alternative methods but not limited to the above procedures.

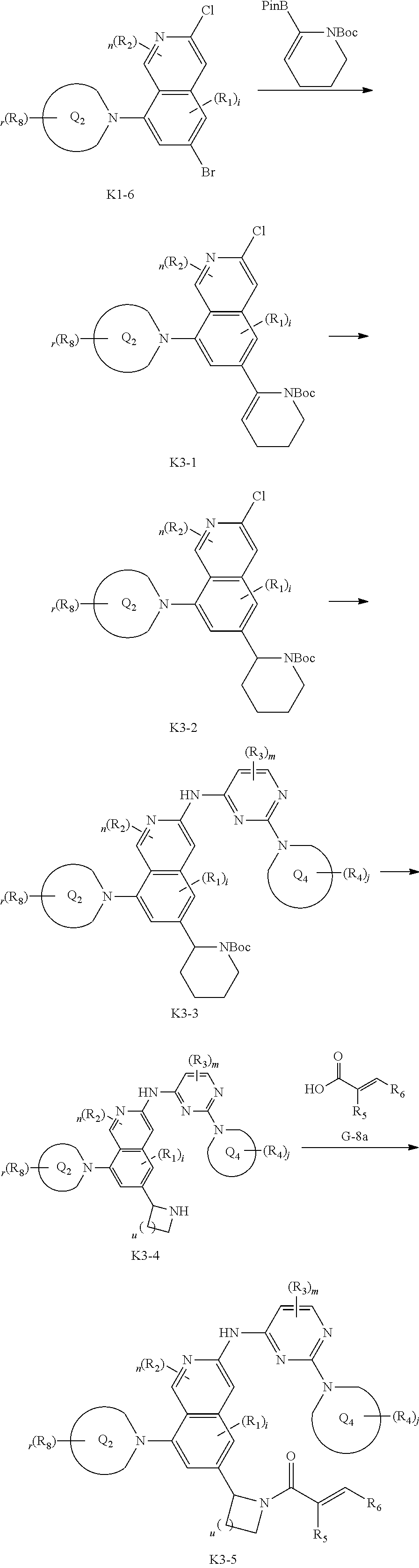

In Scheme K3, the starting material K1-6 can be prepared by conventional procedures using appropriate compounds and reagents. The starting material K1-6 can be converted to K3-1 via Suzuki coupling reaction. After that, the intermediate K3-1 is hydrogenated to yield K3-2, which undergoes Buchwald coupling reaction to yield K3-3. Finally, the intermediate K3-3 is deprotected to give K3-4, which is further treated with G-8a to afford the target compounds K3-5.

Also, the target compounds can be synthesized by alternative methods but not limited to the above procedures.

An approach to synthesize compounds of

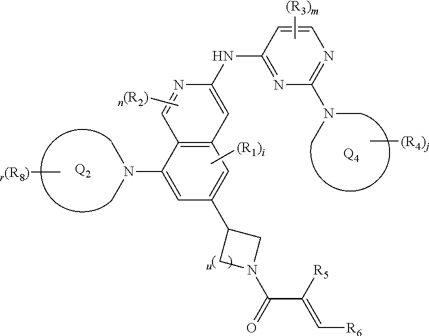

in which u=1, is described in Scheme L1 and u=2, or 3, is described in Scheme L2.

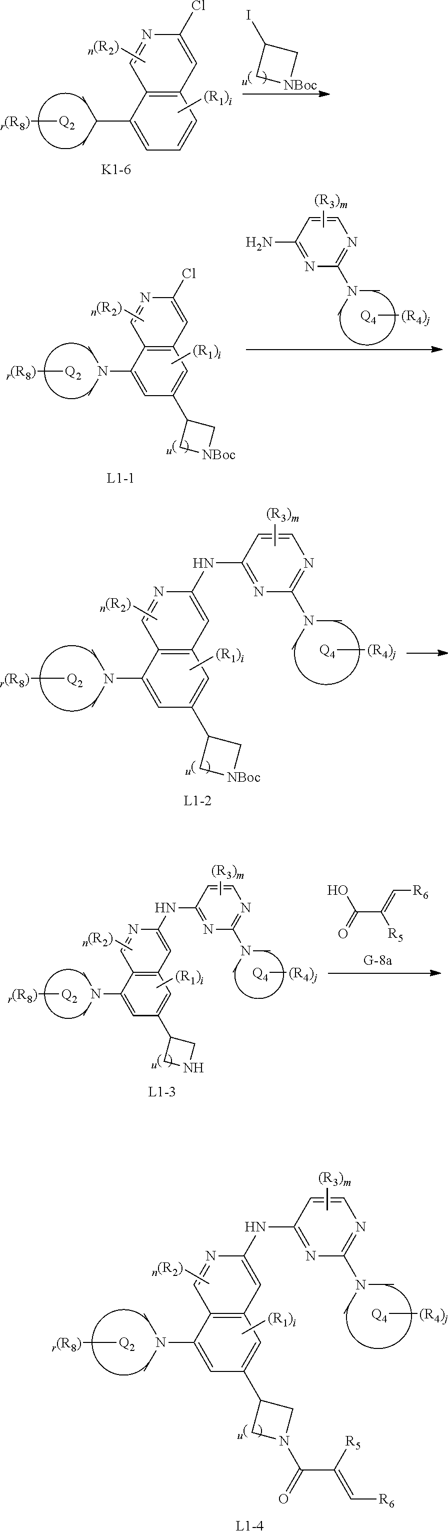

In Scheme L1, the starting material K1-6 can be prepared by conventional procedures using appropriate compounds and reagents. The starting material K1-6 can be converted to L1-1 via Negishi coupling reaction. After that, the intermediate L1-1 undergoes Buchwald coupling reaction to yield L1-2. Finally, the intermediate L1-2 is deprotected to give L1-3, which is further treated with G-8a to afford the target compounds L1-4.

Also, the target compounds can be synthesized by alternative methods but not limited to the above procedures.

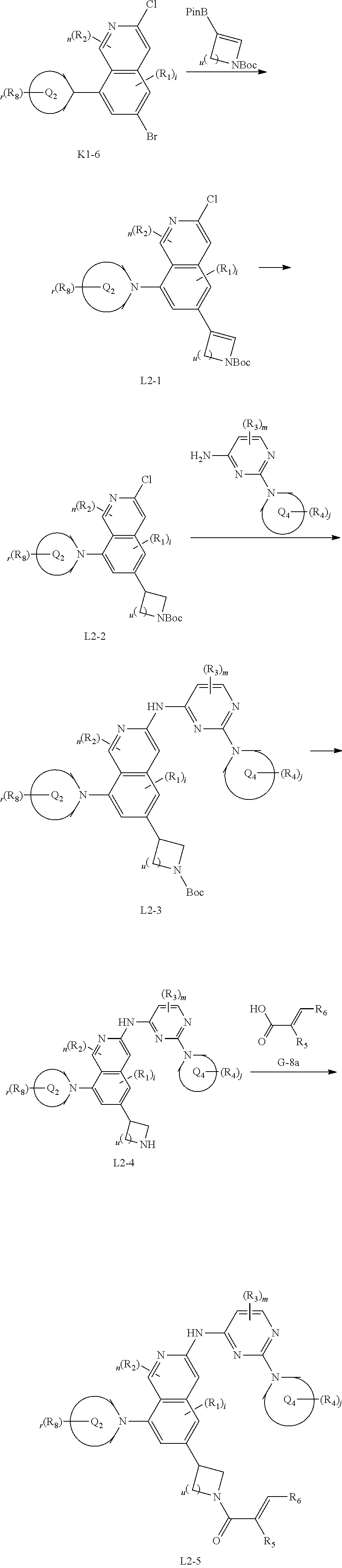

In Scheme L2, the starting material K1-6 can be prepared by conventional procedures using appropriate compounds and reagents. The starting material K1-6 can be converted to L2-1 via Suzuki coupling reaction. After that, the intermediate L2-1 is hydrogenated to yield L2-2, which undergoes Buchwald coupling reaction to yield L2-3. Finally, the intermediate L2-3 is deprotected to give L2-4, which is further treated with G-8a to afford the target compounds L2-5.

Also, the target compounds can be synthesized by alternative methods but not limited to the above procedures.

An approach to synthesize compounds of

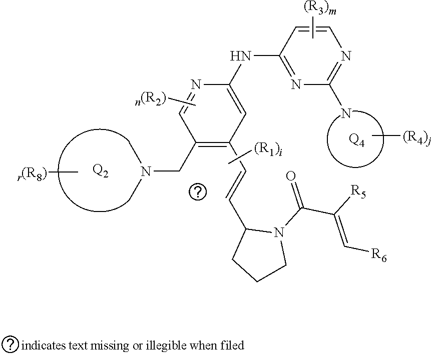

is described in Scheme M.

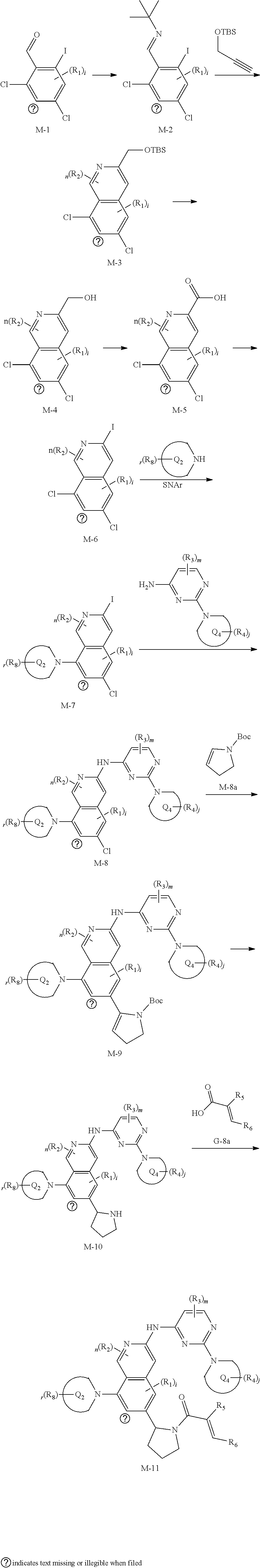

In Scheme M, the starting material M-1 can be prepared by conventional procedures using appropriate compounds and reagents. The starting material M-1 can be converted to M-5 via a literate-known method. After that, the intermediate M-5 is converted to M-6, which can be converted to M-7 through SNAr reaction. Next, the intermediate M-7 undergoes a Buchwald coupling reaction to generate M-8, which react with M-8a to yield M-9. Finally, the intermediate M-9 undergoes a sequence of two-step reaction to give M-10, which is further treated with G-8a to afford the target compounds M-11.

Also, the target compounds can be synthesized by alternative methods but not limited to the above procedures.

An approach to synthesize compounds of

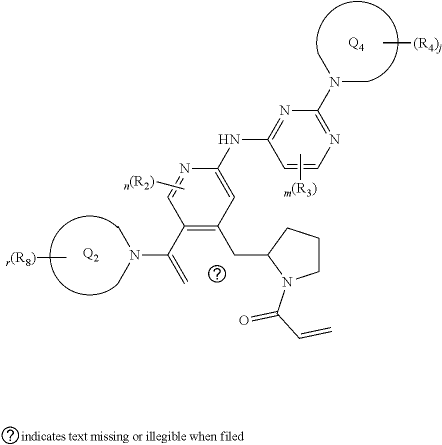

is described in Scheme N.

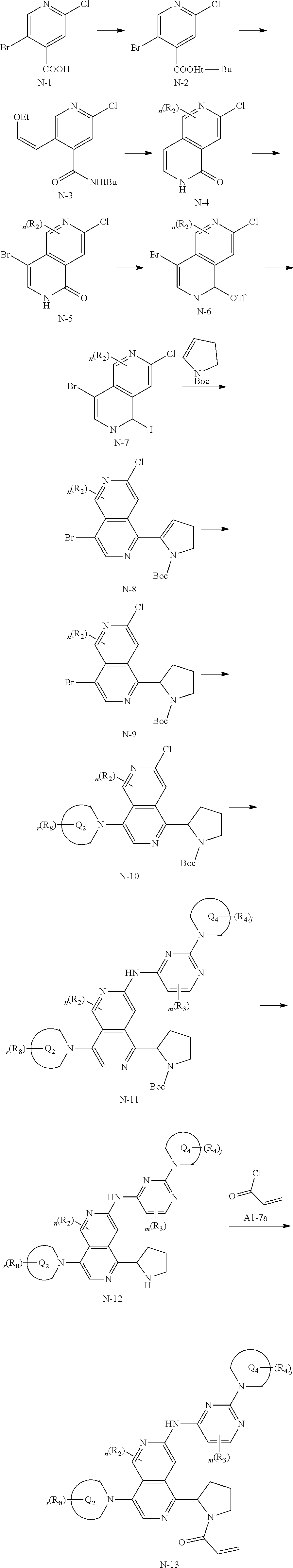

In Scheme N, the starting material N-1 can be prepared by conventional procedures using appropriate compounds and reagents. The starting material N-1 can be converted to N-7 via a literate-known method. After that, the intermediate N-7 is converted to N-8, which is hydrogenated to give N-9. Next, the intermediate N-9 undergoes a Buchwald coupling reaction to generate N-10, which can be converted to N-11 readily. Finally, the intermediate N-11 is deprotected to give N-12, which is further treated with A1-7a to afford the target compounds N-13.

Also, the target compounds can be synthesized by alternative methods but not limited to the above procedures.

An approach to synthesize compounds of

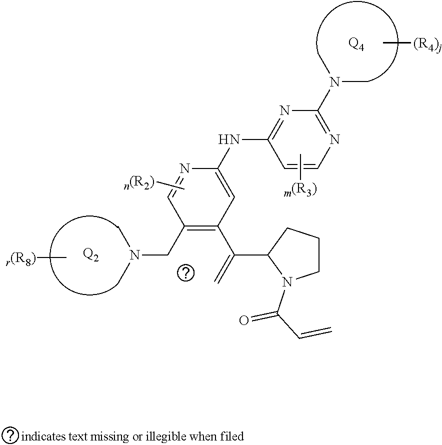

is described in Scheme O.

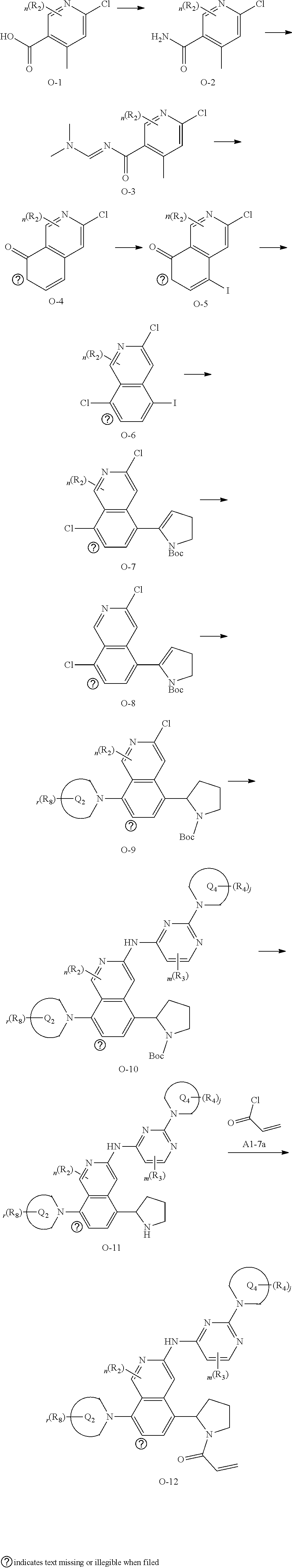

In Scheme O, the starting material O-1 can be prepared by conventional procedures using appropriate compounds and reagents. The starting material O-1 can be converted to O-6 via a literate-known method. After that, the intermediate O-6 is converted to O-7, which is hydrogenated to give O-8. Next, the intermediate O-8 undergoes a SNAr substitution reaction to generate O-9, which can be converted to O-10 readily. Finally, the intermediate O-10 is deprotected to give O-11, which is further treated with A1-7a to afford the target compounds O-12.

Also, the target compounds can be synthesized by alternative methods but not limited to the above procedures.

An approach to synthesize compounds of

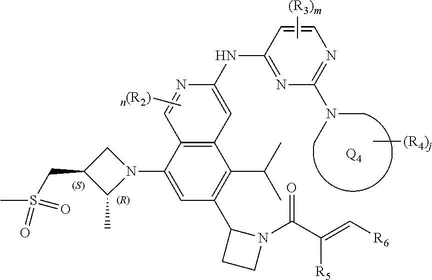

is described in Scheme P.

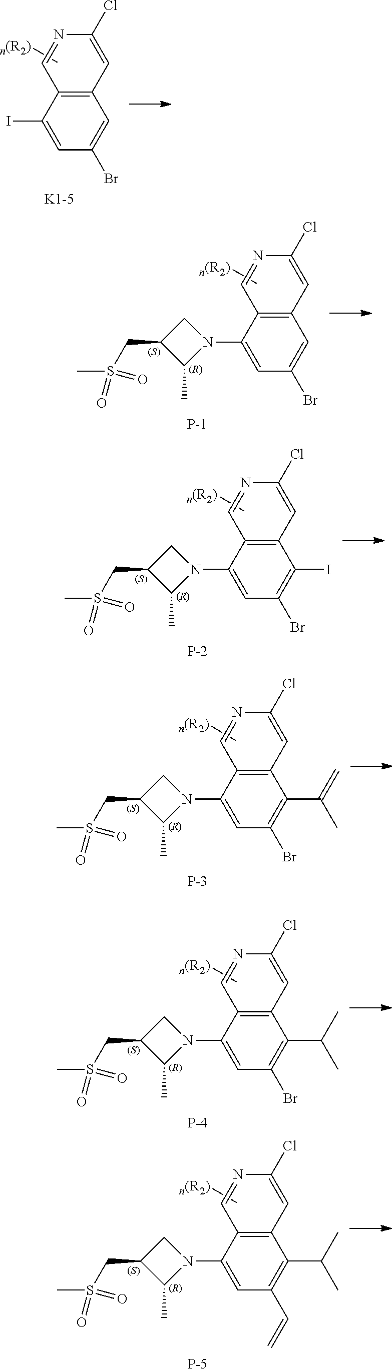

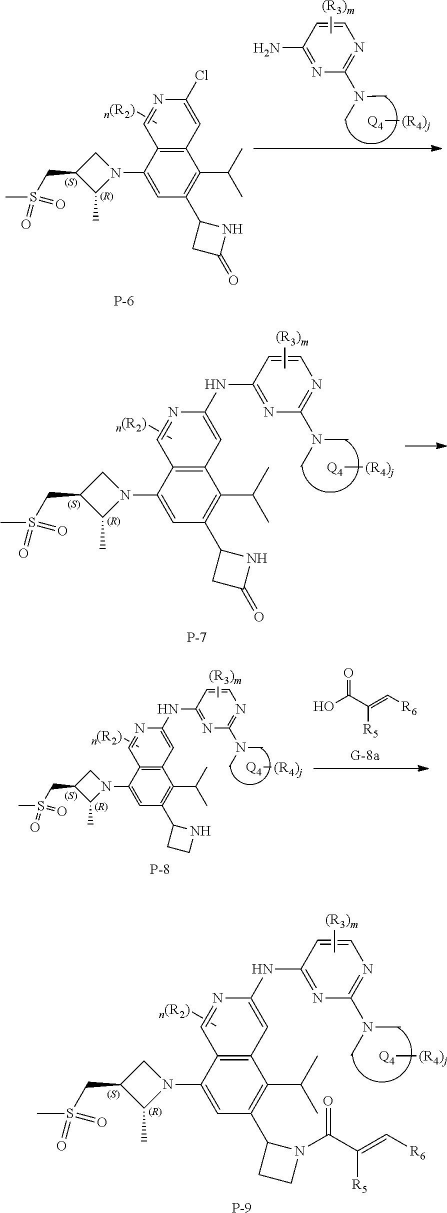

In Scheme P, the starting material K1-5 can be prepared by conventional procedures using appropriate compounds and reagents. The starting material K1-5 is converted to P-1 via Buchwald coupling reaction, which can be converted to P-2. After that, the intermediate P-2 goes through a Suzuki coupling reaction to give P-3, which is hydrogenated to yield P-4. Next, the intermediate P-4 is converted to P-5, which undergoes an intramolecular reaction to yield P-6. Finally, the intermediate P-6 goes through a sequence of two-step reaction to give P-8, which is further treated with G-8a to afford the target compounds P-9.

Also, the target compounds can be synthesized by alternative methods but not limited to the above procedures.

An approach to synthesize compounds of

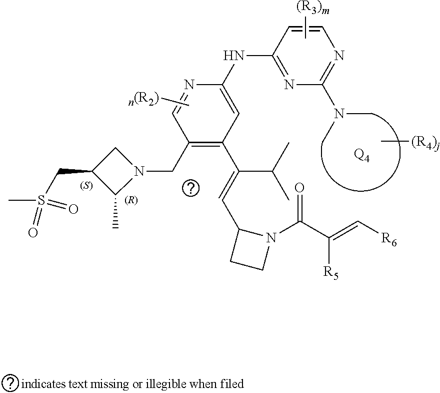

is described in Scheme Q.

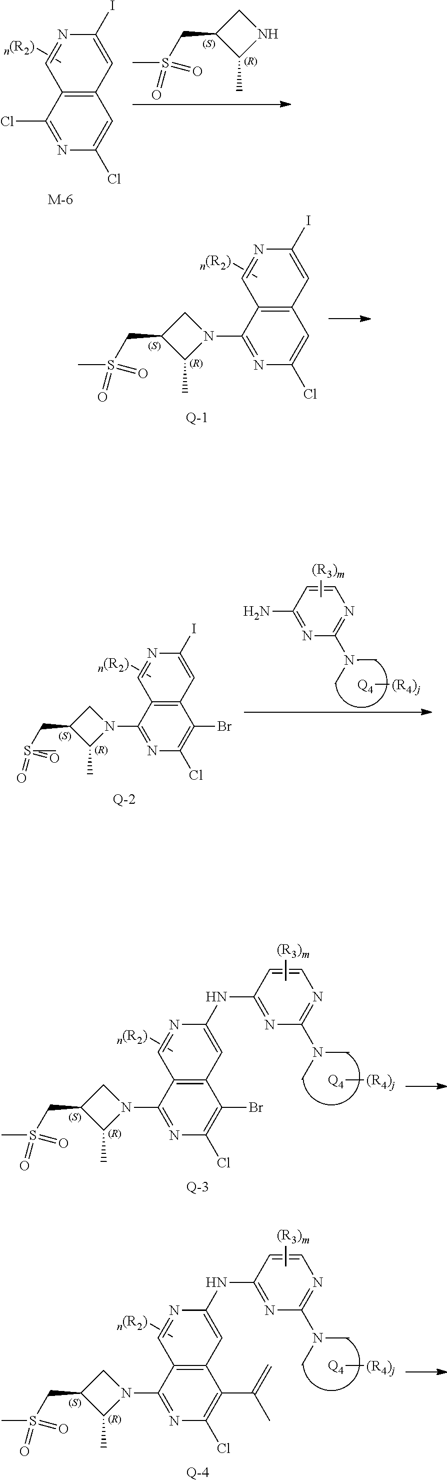

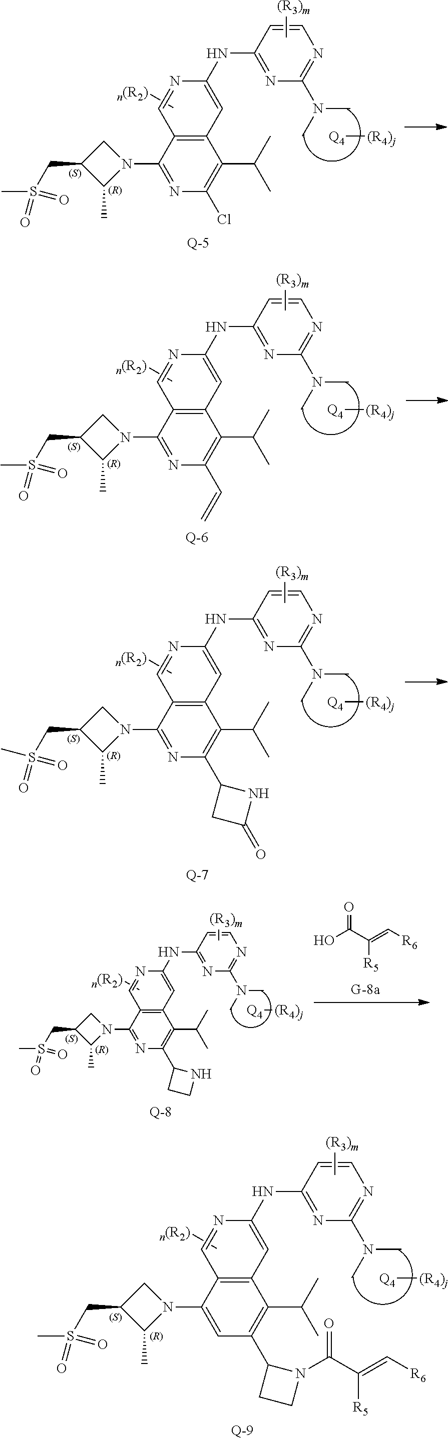

In Scheme Q, the starting material M-6 can be prepared by conventional procedures using appropriate compounds and reagents. The starting material M-6 is converted to Q-1 via a SNAr substitution reaction, which can be converted to bromide Q-2. After that, the intermediate Q-2 goes through Buchwald coupling reaction to give Q-3, which is converted to Q-4 via a Suzuki coupling reaction. Next, the intermediate Q-4 is hydrogenated to yield Q-5, which can be converted to Q-6 readily. The intermediate Q-6 undergoes an intramolecular reaction to yield Q-7. Finally, the intermediate Q-7 is reduced to give Q-8, which is further treated with G-8a to afford the target compounds Q-9.

Also, the target compounds can be synthesized by alternative methods but not limited to the above procedures.

An approach to synthesize compounds of

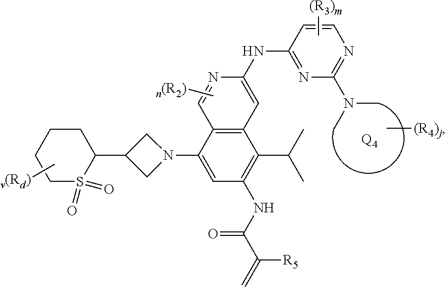

in which v is 1, 2, 3, or 4, is described in Scheme R.

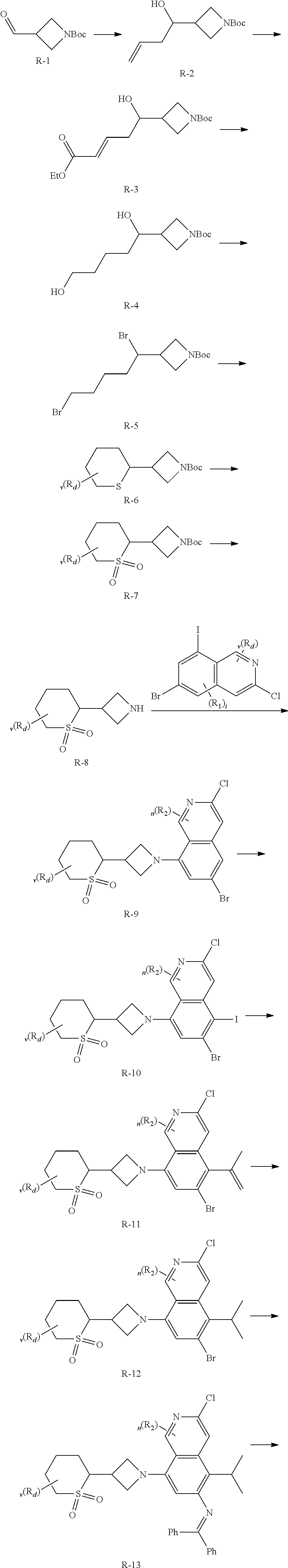

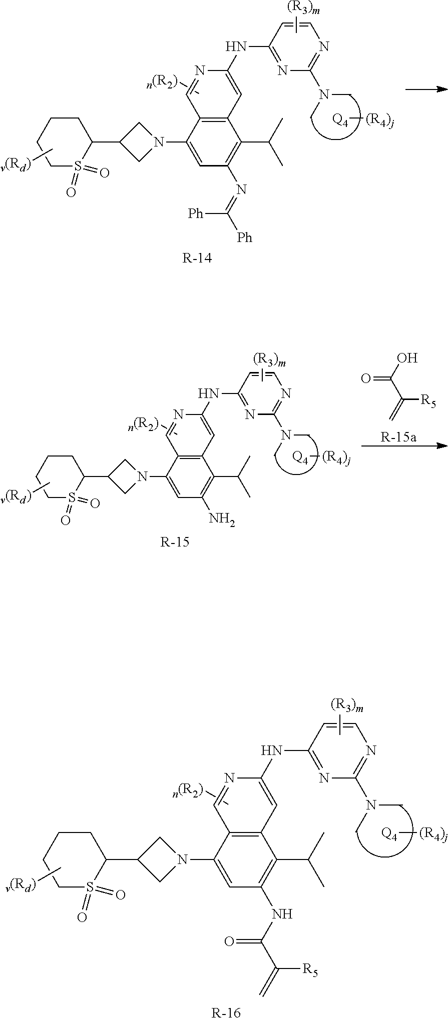

In Scheme R, the starting material R-1 can be prepared by conventional procedures using appropriate compounds and reagents. The starting material R-1 is converted to R-8 via literate-known method. After that, the intermediate R-8 is converted to R-9 via Buchwald coupling reaction, which can be converted to R-10. The intermediate R-10 goes through a Suzuki coupling reaction to give R-11, which is hydrogenated to yield R-12. Next, the intermediate R-12 can be converted to R-13 readily, which undergoes a Buchwald coupling reaction to give R-14. Finally, the intermediate R-14 is deprotected to give R-15, which is further treated with R-15a to afford the target compounds R-16.

Also, the target compounds can be synthesized by alternative methods but not limited to the above procedures.

An approach to synthesize compounds of

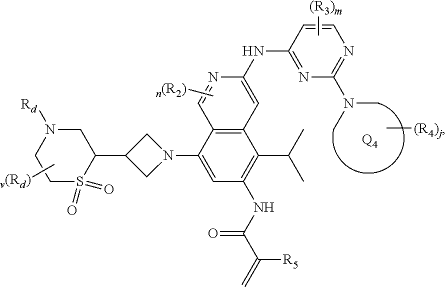

in which v is 1, 2, 3, or 4, is described in Scheme S.

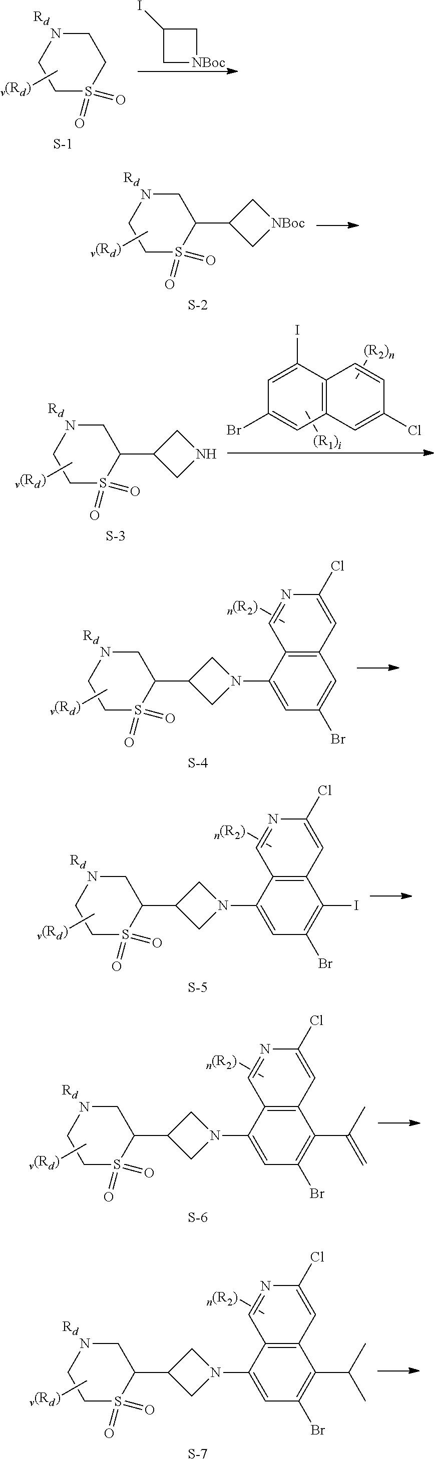

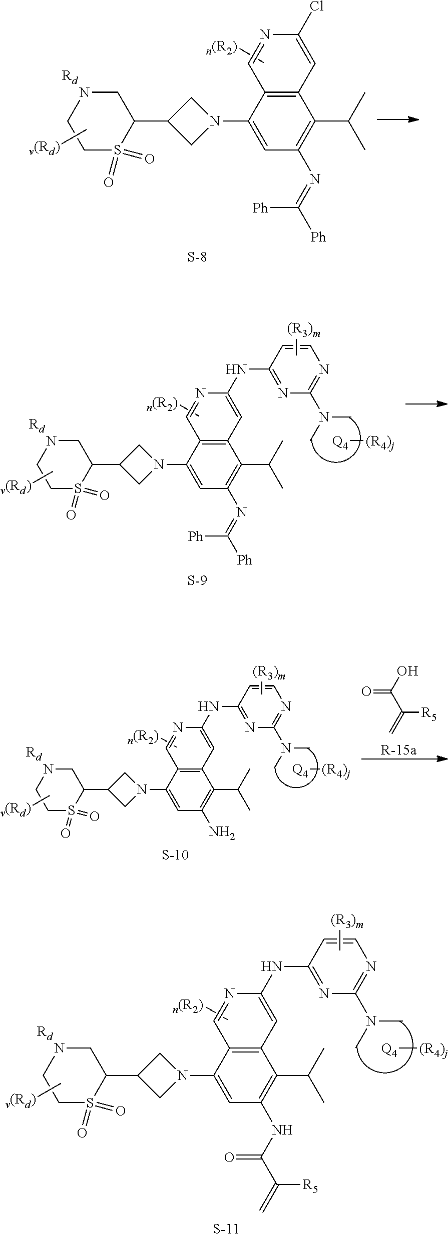

In Scheme S, the starting material S-1 can be prepared by conventional procedures using appropriate compounds and reagents. The starting material S-1 can be converted to S-2, which is deprotected to yield S-3. After that, the intermediate S-3 is converted to S-4 via Buchwald coupling reaction, which can be converted to S 5. The intermediate S-5 goes through a Suzuki coupling reaction to give S-6, which is hydrogenated to yield S-7. Next, the intermediate S-7 can be converted to S-8 readily, which undergoes a Buchwald coupling reaction to give S-9. Finally, the intermediate S-9 is deprotected to give S-10, which is further treated with R-15a to afford the target compounds 5-11.

Also, the target compounds can be synthesized by alternative methods but not limited to the above procedures.

An approach to synthesize compounds of

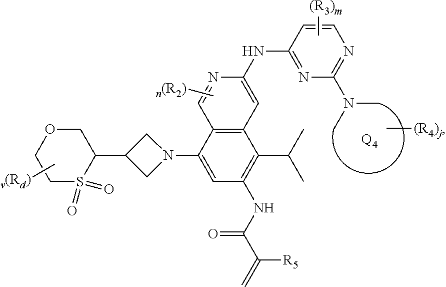

in which v is 1, 2, 3, or 4, is described in Scheme T.

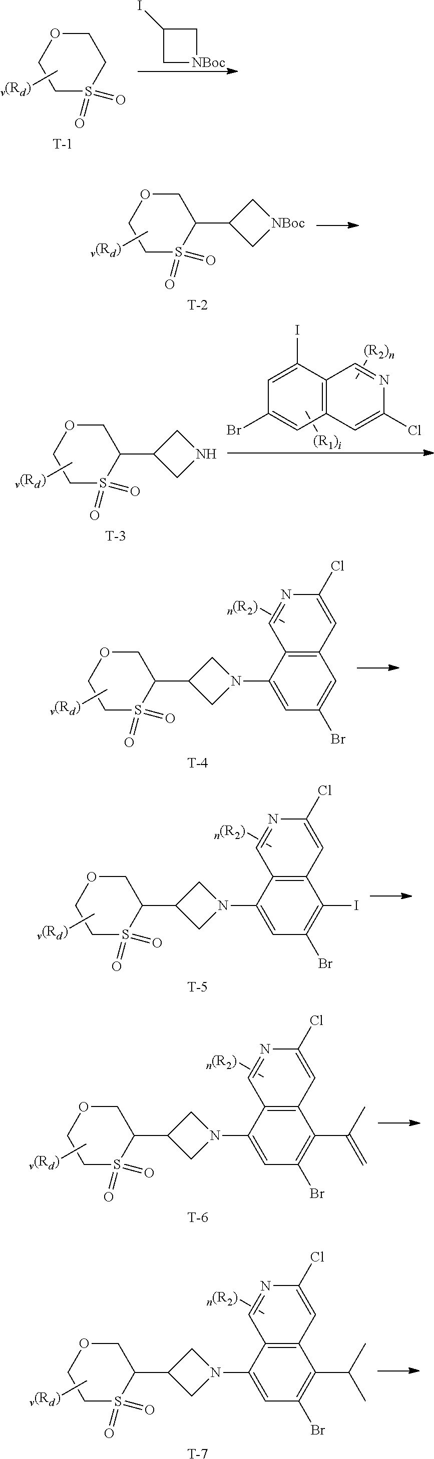

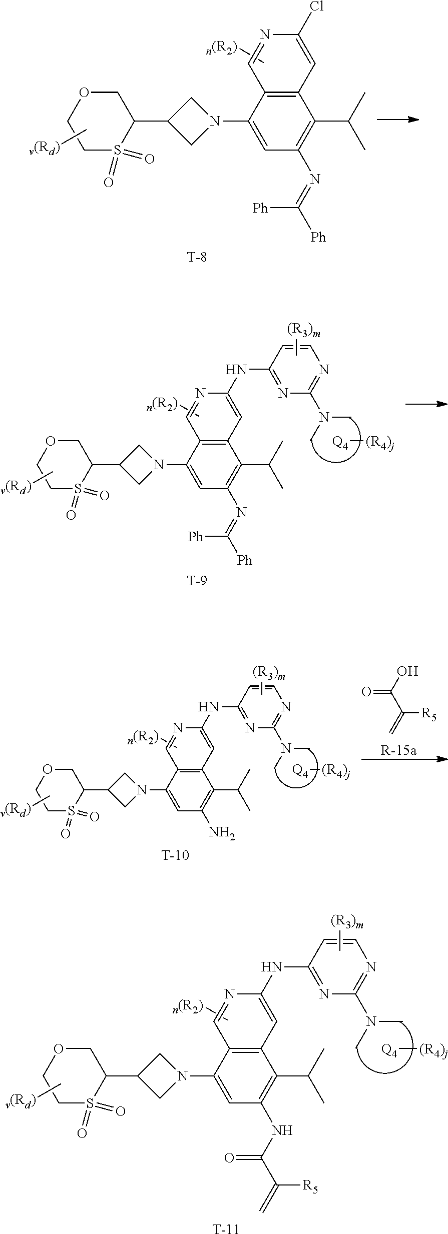

In Scheme T, the starting material T-1 can be prepared by conventional procedures using appropriate compounds and reagents. The starting material T-1 can be converted to T-2, which is deproteced to yield T-3. After that, the intermediate T-3 is converted to T-4 via Buchwald coupling reaction, which can be converted to T-5. The intermediate T-5 goes through a Suzuki coupling reaction to give T-6, which is hydrogenated to yield T-7. Next, the intermediate T-7 can be converted to T-8 readily, which undergoes a Buchwald coupling reaction to give T-9. Finally, the intermediate T-9 is deprotected to give T-10, which is further treated with R-15a to afford the target compounds T-11.

Also, the target compounds can be synthesized by alternative methods but not limited to the above procedures.

An approach to synthesize compounds of

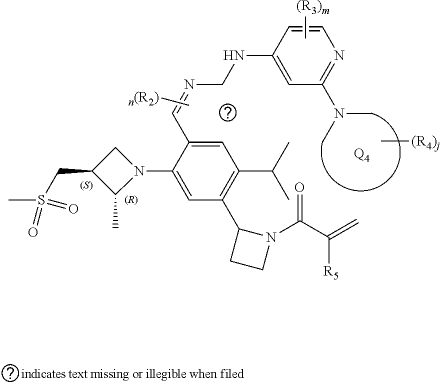

is described in Scheme U.

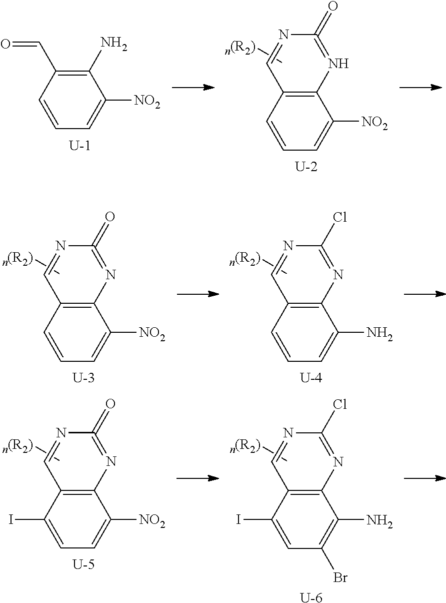

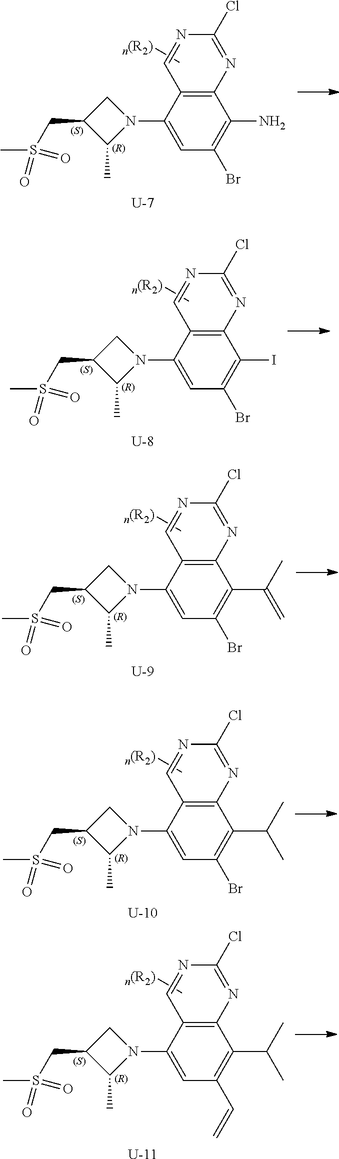

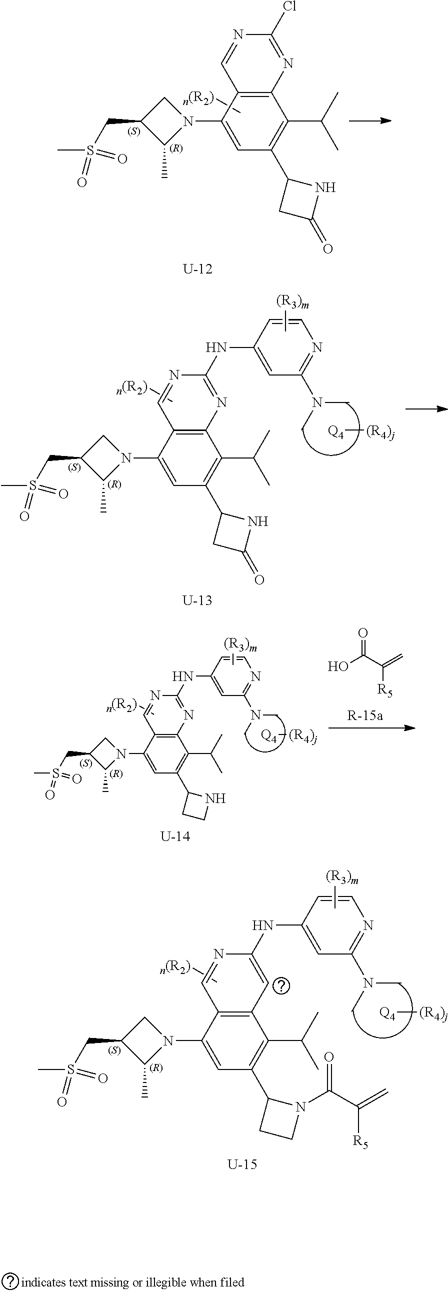

In Scheme U, the starting material U-1 can be prepared by conventional procedures using appropriate compounds and reagents. The starting material U-1 is converted to U-4 via a literate-known method, which can be converted to U-6 through a sequence of two-step halogenated reaction. After that, the intermediate U-6 goes through Buchwald coupling reaction to give U-7, which is converted to U-8 via a Sandmeyer reaction. Next, the intermediate U-8 can be converted to U-10 readily, which can be converted to U-11 via Suzuki coupling reaction. The intermediate U-11 undergoes an intramolecular reaction to yield U-12. Finally, the intermediate U-12 is converted to U-14, which is further treated with R-15a to afford the target compounds U-15.

Also, the target compounds can be synthesized by alternative methods but not limited to the above procedures.

An approach to synthesize compounds of

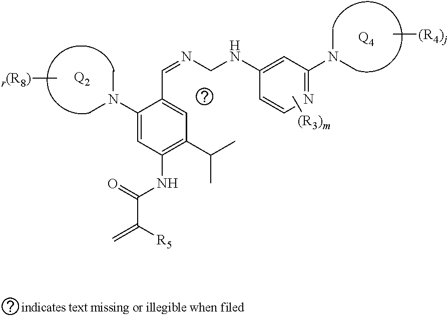

is described in Scheme 1.

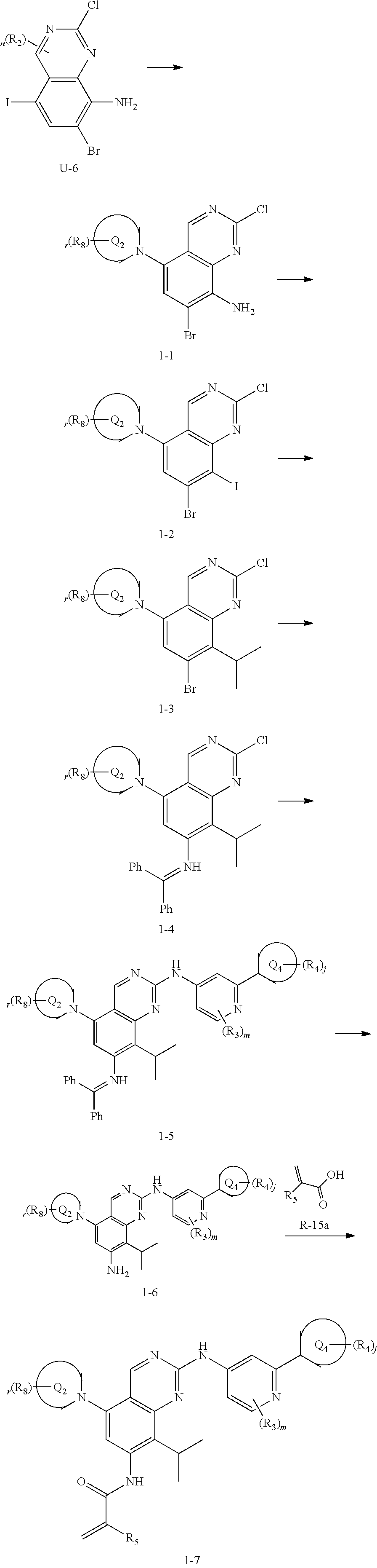

In Scheme 1, the starting material U-6 can be prepared by conventional procedures using appropriate compounds and reagents. The starting material U-6 is converted to 1-1 via Buchwald coupling reaction, which can be converted to 1-2 readily. After that, the intermediate I-2 goes through Negishi coupling reaction to give 1-3, which can be converted to 1-4. Next, the intermediate I-4 can react with an amine to afford 1-5, which is deprotected to give 1-6. Finally, the intermediate I-6 is condensation with R-15a to afford the target compounds 1-7.

Also, the target compounds can be synthesized by alternative methods but not limited to the above procedures.

An approach to synthesize compounds of

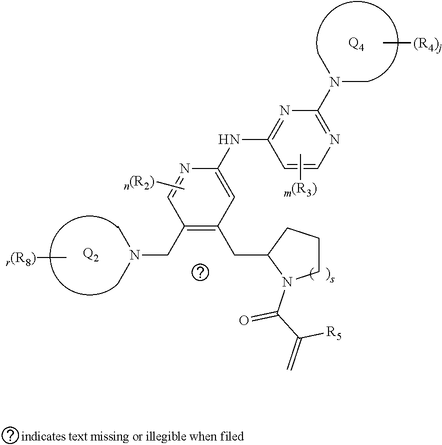

is described in Scheme 2.

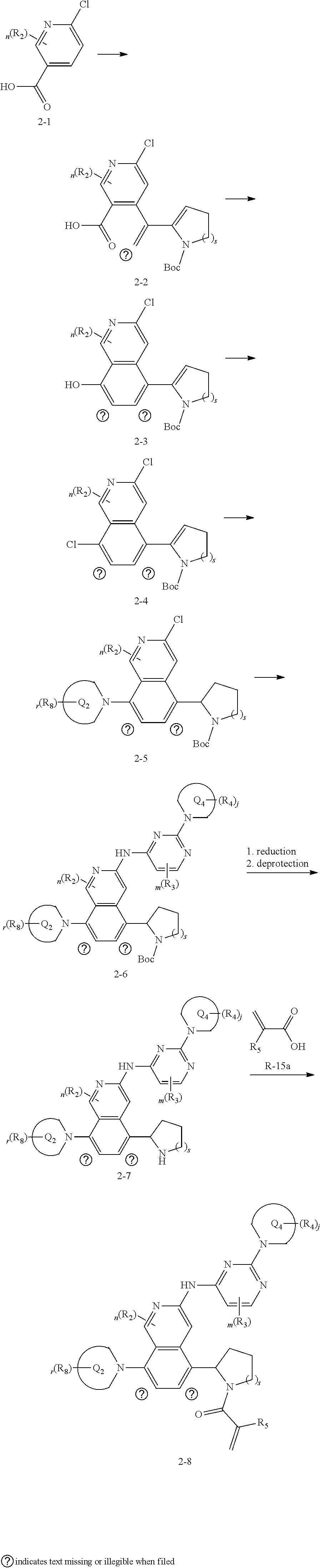

In Scheme 2, the starting material 2-1 can be prepared by conventional procedures using appropriate compounds and reagents. The starting material 2-1 can be converted to 2-3 via a literate known condition. After that, the intermediate 2-3 is converted to chloride 2-4, which goes through a SNAr reaction to provide 2-5. Next, the intermediate 2-5 can couple with an amine to afford 2-6, which goes through a sequence of reduction and deprotection reactions to give 2-7. Finally, the intermediate 2-7 is condensation with R-15a to afford the target compounds 2-8.

Also, the target compounds can be synthesized by alternative methods but not limited to the above procedures.

An approach to synthesize compounds of

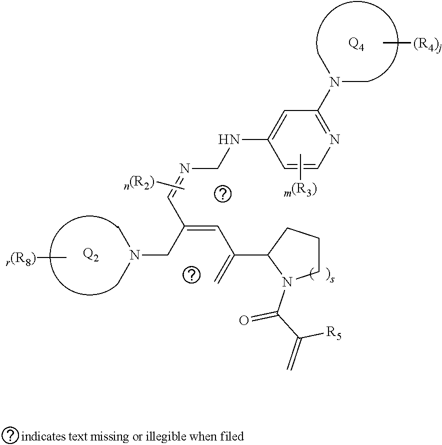

is described in Scheme 3.

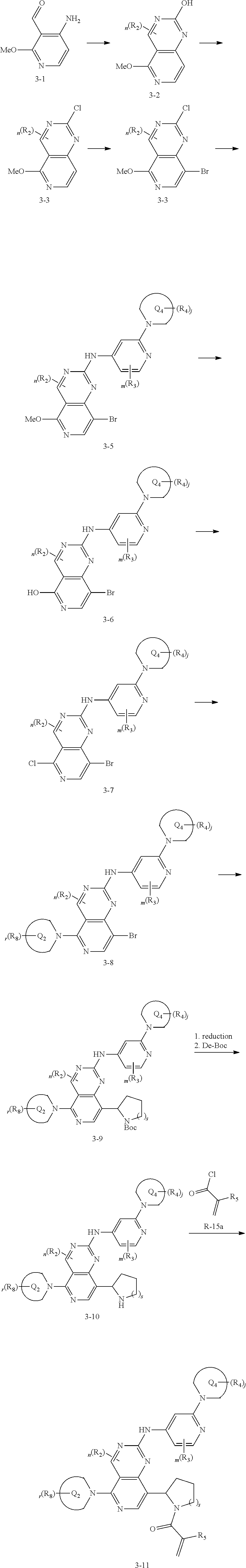

In Scheme 3, the starting material 3-1 can be prepared by conventional procedures using appropriate compounds and reagents. The starting material 3-1 can be converted to 3-2 via a literate known condition. After that, the intermediate 3-2 is converted to chloride 3-3, which goes through a bromination reaction to provide 3-4. The intermediate 3-4 can react with an amine to afford 3-5, which is demethylated to give 3-6. Next, the intermediate 3-6 is converted to 3-7, which can be converted to 3-9 via a sequence of SNAr and Heck coupling reactions. The intermediate 3-9 can further goes though a sequence of reduction and deprotection reactions to provide 3-10. Finally, 3-10 is condensation with R-15a to afford the target compounds 3-11.

Also, the target compounds can be synthesized by alternative methods but not limited to the above procedures.

An approach to synthesize compounds of

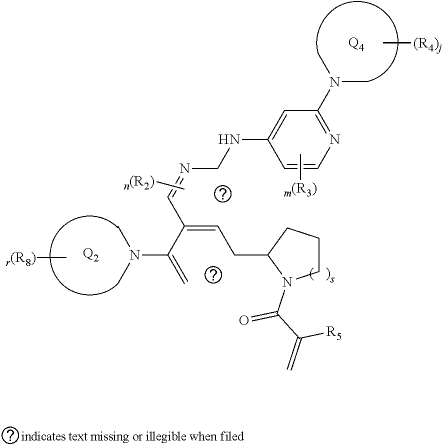

is described in Scheme 4.

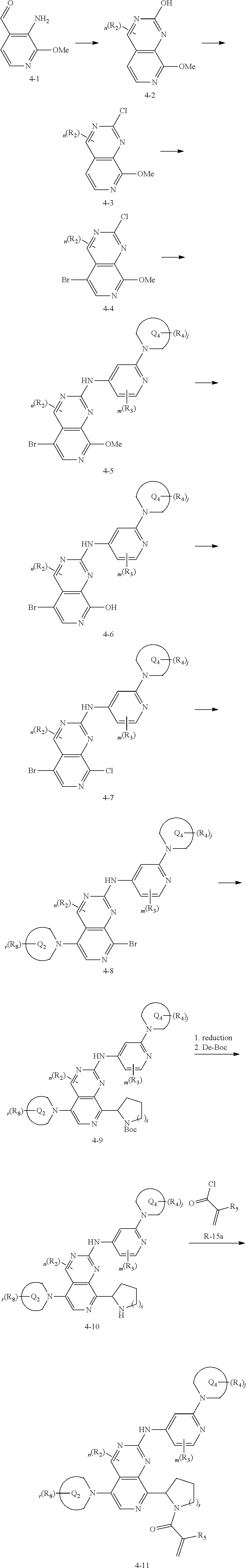

In Scheme 4, the starting material 4-1 can be prepared by conventional procedures using appropriate compounds and reagents. The starting material 4-1 can be converted to 4-2 via a literate known condition. After that, the intermediate 4-2 is converted to chloride 4-3, which goes through a bromination reaction to provide 4-4. The intermediate 4-4 can react with an amine to afford 4-5 through SNAr reaction, which is demethylated to give 4-6. Next, the intermediate 4-6 is converted to 4-7, which can be converted to 4-9 via a sequence of Buchwald and Heck coupling reactions. The intermediate 4-9 can further goes though a sequence of reduction and deprotection reactions to provide 4-10. Finally, the intermediate 4-10 is condensation with R-15a to afford the target compounds 4-11.

Also, the target compounds can be synthesized by alternative methods but not limited to the above procedures.

The compounds of

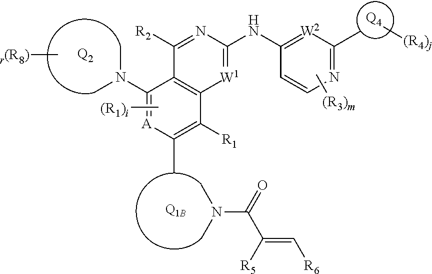

can be made by the method referred to Scheme A-U and Scheme 1-4 by using different starting material, intermediates, and reagents.

The compounds of

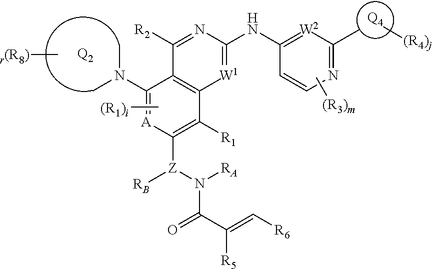

can be made by the method referred to Scheme A-U and Scheme 1-4 by using different starting material, intermediates, and reagents.

The compounds of

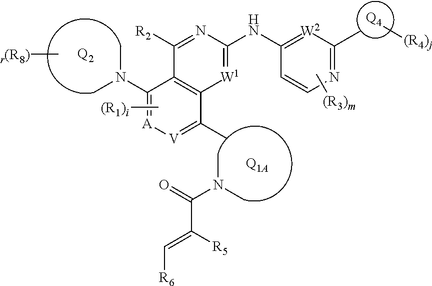

can be made by the method referred to Scheme A-U and Scheme 1-4 by using different starting material, intermediates, and reagents.

The compounds of

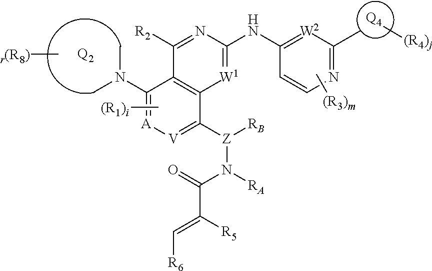

can be made by the method referred to Scheme A-U and Scheme 1-4 by using different starting material, intermediates, and reagents.

The compounds of

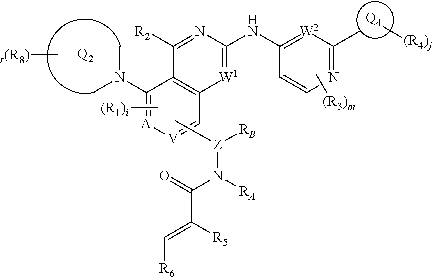

can be made by the method referred to Scheme A-U and Scheme 1-4 by using different starting material, intermediates, and reagents.

The compounds of

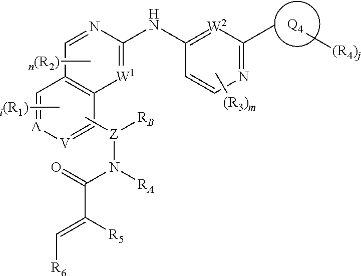

can be made by the method referred to Scheme A-U and Scheme 1-4 by using different starting material, intermediates, and reagents.

The compounds of

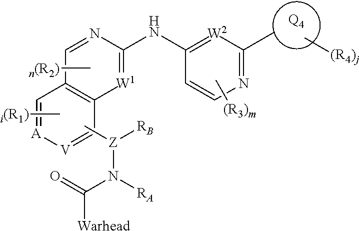

can be made by the method referred to Scheme A-U and Scheme 1-4 by using different starting material, intermediates, and reagents.

The compounds of

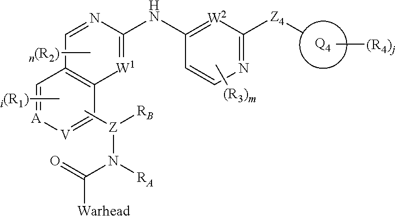

can be made by the method referred to Scheme A-U and Scheme 1-4 by using different starting material, intermediates, and reagents.

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### Example INT-1: Preparation of (2R,3S)-3-(methanesulfonylmethyl)-2-methylazetidine trifluoroacetic acid salt (racemate)

Synthesis of [(2S,3S)-3-methyloxiran-2-yl]methanol: Into a 2 L round-bottom flask, were placed crotyl alcohol (25.0 g, 346.7 mmol, 1.0 eq), DCM (1 L). After that, m-CPBA (119.7 g, 693.4 mmol, 2.0 eq) was added in portions to the above mixture at 25° C. The resulting mixture was stirred for 18 hours at 25° C. The reaction mixture was concentrated to ¼ volume under vacuum. Then the solid was filtered out and the filtrate was used directly in next step. LC-MS (ES, m/z) M+1: 89.

Synthesis of [(2S,3S)-3-methyloxiran-2-yl]methyl 4-nitrobenzoate: To the above mixture were added triethylamine (103.4 g, 1021.5 mmol, 3.0 eq) and 4-nitro-benzoyl chloride (75.8 g, 408.6 mmol, 1.2 eq) in portions at 0° C. The resulting mixture was stirred for additional 1 hour at 5° C. The reaction was quenched by the addition of water (100 mL) and then extracted with dichloromethane (2×100 mL). The combined organic layers were dried over anhydrous Na₂SO₄. After filtration, the filtrate was concentrated under vacuum. The residue was purified by a flash column (silica gel, ethyl acetate/petroleum ether=1:8) to afford [(2S,3S)-3-methyloxiran-2-yl]methyl-4-nitrobenzoate as a white solid (38.0 g, 47.1%). LC-MS (ES, m/z) M+1: 238. ¹HNMR (300 MHz, Chloroform-d) δ 8.37-8.27 (m, 2H), 8.31-8.12 (m, 2H), 4.71 (dd, J=12.3, 3.0 Hz, 1H), 4.22 (dd, J=12.2, 6.4 Hz, 1H), 3.16-2.99 (m, 2H), 1.41 (d, J=5.1 Hz, 3H).

Synthesis of (2R,3R)-3-[(diphenylmethyl)amino]butane-1,2-diol: Into a 2 L round-bottom flask, were placed [(2S,3S)-3-methyloxiran-2-yl]methyl 4-nitrobenzoate (38.0 g, 160.2 mmol, 1.0 eq), titanium isopropoxide (77.0 g, 320.3 mmol, 2.0 eq) and DCM (700 mL). After stirring for 10 min, aminodiphenylmethane (58.7 g, 320.4 mmol, 2.0 eq) was added at 25° C. The resulting mixture was stirred for 16 hours at 25° C. The resulting mixture was diluted with dichloromethane (700 mL) and quenched by the addition of 10% aq. NaOH (80 mL). The resulting mixture was stirred for additional 2 hours and then filtered. The filtrate was concentrated under vacuum. The residue was purified by a flash column (silica gel, ethyl acetate/petroleum ether=1:2) to afford (2R,3R)-3-[(diphenylmethyl)amino]butane-1,2-diol as a white solid (17.0 g, 39.1%). LC-MS (ES, m/z) M+1: 272. ¹HNMR (300 MHz, DMSO-d₆) δ 7.48-7.12 (m, 12H), 5.01 (s, 1H), 4.53 (d, J=5.0 Hz, 1H), 3.56 (dt, J=13.7, 6.7 Hz, 1H), 3.36 (d, J=6.1 Hz, 2H), 2.17 (s, 1H), 0.96 (d, J=6.5 Hz, 3H).

Synthesis of (2R,3S)-1-(diphenylmethyl)-2-methylazetidin-3-ol: Into a 250 mL round-bottom flask, were placed (2R,3R)-3-[(diphenylmethyl)amino]butane-1,2-diol (4.9 g, 18.1 mmol, 1 eq), triethylamine (2.7 g, 27.1 mmol, 1.5 eq), DCM (100 mL). After that, methylsufonyl chloride (2.5 g, 21.7 mmol, 1.2 eq) was added dropwise at 0° C. The resulting mixture was stirred for additional 2 hours at 25° C. The reaction was quenched by the addition of water (20 mL) and extracted with dichloromethane (2×50 mL). The combined organic layers were dried over anhydrous Na₂SO₄. After filtration, the filtrate was concentrated under vacuum to afford (2R,3S)-1-(diphenylmethyl)-2-methylazetidin-3-ol as a yellow semi-solid (3.8 g, 83.1%). LC-MS (ES, m/z) M+1: 254. ¹HNMR (300 MHz, DMSO-d₆) δ 7.41 (dt, J=8.0, 1.5 Hz, 4H), 7.35-7.12 (m, 6H), 5.26 (d, J=6.4 Hz, 1H), 4.36 (s, 1H), 3.67 (p, J=6.4 Hz, 1H), 3.43 (t, J=6.6 Hz, 1H), 2.91 (p, J=6.2 Hz, 1H), 2.43 (t, J=6.9 Hz, 1H), 0.62 (d, J=6.2 Hz, 3H).

Synthesis of (2R,3S)-1-(diphenylmethyl)-2-methylazetidin-3-yl methanesulfonate: Into a 250 mL round-bottom flask, were placed (2R,3S)-1-(diphenylmethyl)-2-methylazetidin-3-ol (3.3 g, 13.0 mmol, 1.0 eq), triethylamine (4.0 g, 39.1 mmol, 3.0 eq), DCM (70 mL). After that, methanesulfonyl chloride (2.2 g, 19.5 mmol, 1.5 eq) was added dropwise at 0° C. The resulting mixture was stirred for additional 3 hours at 25° C. The reaction was quenched by the addition of water (30 mL) and extracted with dichloromethane (2×50 mL). The combined organic layers were dried over anhydrous Na₂SO₄. After filtration, the filtrate was concentrated under vacuum to afford (2R,3S)-1-(diphenylmethyl)-2-methylazetidin-3-yl methanesulfonate as a yellow semi-solid (4.2 g, 97.3%). LC-MS (ES, m/z) M+1: 332. ¹HNMR (300 MHz, DMSO-d₆) δ 7.43 (dt, J=8.0, 1.6 Hz, 4H), 7.37-7.23 (m, 4H), 7.29-7.15 (m, 3H), 4.64 (q, J=6.2 Hz, 1H), 4.54 (s, 1H), 3.61 (ddd, J=7.7, 6.4, 1.1 Hz, 1H), 3.33 (d, J=12.8 Hz, 1H), 3.18 (s, 3H), 2.84-2.73 (m, 1H), 1.27-1.13 (m, 1H), 0.72 (d, J=6.2 Hz, 3H).

Synthesis of ethyl 2-[(2R,3S)-1-(diphenylmethyl)-2-methylazetidin-3-yl]-2-methanesulfonylacetate: Into a 250 mL round-bottom flask, were placed ethyl 2-methanesulfonylacetate (2 g, 11.9 mmol, 1.2 eq), DMF (70 mL). After that, NaH (360 mg, 14.9 mmol, 1.5 eq) was added in portions at 0° C. The resulting mixture was stirred for additional 30 min at 0° C. and then a solution of (2R,3S)-1-(diphenylmethyl)-2-methylazetidin-3-yl methanesulfonate (3.3 g, 10.0 mmol, 1.0 eq) in DMF (10 mL) was added dropwise at 0° C. The resulting mixture was stirred for additional 16 hours at 80° C. The reaction was quenched by the addition of water (80 mL) and extracted with ethyl acetate (2×30 mL). The combined organic layers were washed with brine (50 mL), dried over anhydrous Na₂SO₄. After filtration, the filtrate was concentrated under vacuum. The residue was purified by a flash column (silica gel, ethyl acetate/petroleum ether=1:4) to afford ethyl 2-[(2R,3S)-1-(diphenylmethyl)-2-methylazetidin-3-yl]-2-methanesulfonylacetate as a white solid (1.8 g, 45.0%). LC-MS (ES, m/z) M+1: 402. ¹HNMR (300 MHz, DMSO-d₆) δ 7.41 (d, J=7.5 Hz, 5H), 7.36-7.25 (m, 3H), 7.30-7.14 (m, 4H), 4.53 (d, J=20.7 Hz, 2H), 4.17 (q, J=7.1 Hz, 2H), 3.48-3.35 (m, 1H), 3.06 (d, J=5.7 Hz, 3H), 2.80 (t, J=8.0 Hz, 1H), 2.72-2.52 (m, 1H), 1.19 (td, J=7.1, 3.0 Hz, 3H), 0.61 (d, J=6.0 Hz, 2H).

Synthesis of (2R,3S)-1-(diphenylmethyl)-3-(methanesulfonylmethyl)-2-methylazetidine: Into a 50 mL round-bottom flask, were placed ethyl 2-[(2R,3S)-1-(diphenylmethyl)-2-methylazetidin-3-yl]-2-methanesulfonylacetate (1.8 g, 4.5 mmol, 1.0 eq), LiCl (1.5 g, 35.9 mmol, 8.0 eq), N, N-dimethylacetamide (20 mL). The resulting mixture was stirred for additional 2 hours at 150° C. The resulting mixture was diluted with water (40 mL) and extracted with ethyl acetate (3×20 mL). The combined organic layers were washed with brine, dried over anhydrous Na₂SO₄. After filtration, the filtrate was concentrated under vacuum. The residue was purified by a flash column (silica gel, ethyl acetate/petroleum ether=1:5) to afford (2R,3S)-1-(diphenylmethyl)-3-(methanesulfonylmethyl)-2-methylazetidine as a white solid (1.1 g, 71.1%). LC-MS (ES, m/z) M+1: 330. ¹HNMR (300 MHz, DMSO-d₆) δ 7.47-7.37 (m, 4H), 7.35-7.21 (m, 4H), 7.19 (td, J=7.3, 1.2 Hz, 2H), 4.44 (s, 1H), 3.47-3.24 (m, 3H), 3.08 (q, J=6.4 Hz, 1H), 2.90 (s, 3H), 2.57 (dd, J=8.5, 7.2 Hz, 1H), 2.40 (h, J=7.8 Hz, 1H), 0.70 (d, J=6.0 Hz, 3H).

Synthesis of (2R,3S)-3-(methanesulfonylmethyl)-2-methylazetidine trifluoroacetic acid salt: Into a 50 mL round-bottom flask, were placed (2R,3S)-1-(diphenylmethyl)-3-(methanesulfonylmethyl)-2-methylazetidine (1.1 g, 3.0 mmol, 1.0 eq), Pd(OH)₂/C (0.2 g, 1.5 mmol, 0.5 eq), trifluoroacetic acid (0.5 mL), MeOH (15 mL). The resulting mixture was stirred for additional 16 hours at 25° C. under an atmosphere of H₂ balloon. The solid was filtered out, and the filtrate was concentrated under vacuum. The crude product was slurried with diethyl ether to afford (2R,3S)-3-(methanesulfonylmethyl)-2-methylazetidine(racemate) trifluoroacetic acid salt as a yellow solid (0.8 g, 95.0%). LC-MS (ES, m/z) M-1:164. ¹HNMR (300 MHz, DMSO-d₆) δ 4.31 (p, J=6.8 Hz, 1H), 3.97-3.86 (m, 1H), 3.79 (t, J=7.8 Hz, 1H), 3.57 (dd, J=12.6, 5.9 Hz, 1H), 3.53 (d, J=4.5 Hz, 1H), 3.01 (s, 4H), 1.45 (d, J=6.7 Hz, 3H).

### Example INT-2: Preparation of Synthesis of 8-bromo-3-chloro-5-methoxyisoquinoline

Synthesis of (2E)-2-(hydroxyimino)-4-methoxy-3H-inden-1-one: Into a 5 L round-bottom flask, were placed 4-methoxy-2,3-dihydroinden-1-one (95.0 g, 585.7 mmol, 1.0 eq), isopentyl nitrite (102.9 g, 878.6 mmol, 1.5 eq), THE (1.7 L). After that, HCl in diethyl ether (100 mL, 2 M) was added dropwise at 0° C. and then the mixture was stirred for 2 hours at 0° C. The resulting mixture was concentrated to ⅓ volume under vacuum. The precipitate was collected by filtration, and then the filter cake was dired under vacuum to afford (2E)-2-(hydroxyimino)-4-methoxy-3H-inden-1-one as a light yellow solid (106.0 g, 94.7%). LC-MS (ES, m/z) M+1: 192. ¹HNMR (300 MHz, DMSO-d₆) δ 12.65 (s, 1H), 7.45 (dd, J=8.5, 7.0 Hz, 1H), 7.31 (d, J=7.8 Hz, 2H), 3.89 (s, 3H), 3.58 (s, 2H).

Synthesis of 1,3-dichloro-5-methoxyisoquinoline: Into a 5 L round-bottom flask, were placed (2E)-2-(hydroxyimino)-4-methoxy-3H-inden-1-one (101.0 g, 528.2 mmol, 1.0 eq), POCl₃ (15400 mL). After that, PCl₅ (132.0 g, 633.9 mmol, 1.2 eq) and HCl in dioxane (500 mL, 4 M) were added dropwise at 0° C. The resulting mixture was stirred for additional 16 hours at 60° C. The resulting mixture was concentrated under vacuum and then quenched by the addition of ice water. The precipitated solids were collected by filtration and washed with ice water to afford 1,3-dichloro-5-methoxyisoquinoline as a white solid (80.0 g, 66.4%). LC-MS (ES, m/z) M+1: 228/230. ¹HNMR (300 MHz, DMSO-d₆) δ 8.03 (s, 1H), 7.82 (d, J=8.4 Hz, 1H), 7.76 (t, J=7.9 Hz, 1H), 7.41 (d, J=7.4 Hz, 1H), 4.03 (s, 3H).

Synthesis of 3-chloro-5-methoxyisoquinoline: Into a 5 L round-bottom flask, were placed 1,3-dichloro-5-methoxyisoquinoline (80.0 g, 350.8 mmol, 1.0 eq), HI (1400 mL, 45%), acetic acid (2800 mL). After that, red phosphorus (27.2 g, 876.9 mmol, 2.5 eq) was added in portions at 25° C. The resulting mixture was stirred for additional 4 hours at 100° C. The resulting mixture was concentrated under vacuum. The residue diluted with dichloromethane (3 L) and then washed with aq. NaHCO₃ (1 L), dried over anhydrous Na₂SO₄. After filtration, the filtrate was concentrated under vacuum to afford 3-chloro-5-methoxyisoquinoline as a red solid (60.0 g, 88.3%). LC-MS (ES, m/z) M+1: 194/196. ¹HNMR (400 MHz, DMSO-d₆) δ 9.17 (d, J=1.0 Hz, 1H), 7.96 (q, J=0.9 Hz, 1H), 7.71 (dd, J=8.3, 1.0 Hz, 1H), 7.67-7.59 (m, 1H), 7.28 (dd, J=7.7, 1.2 Hz, 1H), 4.00 (s, 3H).

Synthesis of 8-bromo-3-chloro-5-methoxyisoquinoline: Into a 2 L round-bottom flask, were placed 3-chloro-5-methoxyisoquinoline (60.0 g, 309.7 mmol, 1.0 eq), acetic acid (500 mL). After that, a solution of Br₂ (56.9 g, 356.3 mmol, 1.2 eq) in acetic acid (250 mL) was added dropwise at 25° C. The resulting mixture was stirred for additional 2 days at 25° C. The resulting mixture was added dropwise to a stirred aq. K₂CO₃ solution at 0° C. The precipitated were collected by filtration and washed with ice water. The residue was diluted with dichloromethane (3 L), dried over anhydrous Na₂SO₄. After filtration, the filtrate was concentrated under vacuum to afford 8-bromo-3-chloro-5-methoxyisoquinoline as a red solid (70.0 g, 87.4%). LC-MS (ES, m/z) M+1: 272/274. ¹HNMR (400 MHz, DMSO-d₆) δ 9.22 (d, J=0.9 Hz, 1H), 8.00 (d, J=0.8 Hz, 1H), 7.91 (d, J=8.3 Hz, 1H), 7.20 (d, J=8.3 Hz, 1H), 4.01 (s, 3H).

### Example INT-3: Preparation of Synthesis of 8-bromo-3-chloroisoquinolin-5-yl trifluoromethanesulfonate

Synthesis of 8-bromo-3-chloroisoquinolin-5-ol: Into a 2 L round-bottom flask, were placed 8-bromo-3-chloro-5-methoxyisoquinoline (70.0 g, 256.9 mmol, 1 eq), BBr₃ (1 M in DCM, 700 mL). The resulting mixture was stirred for 2 days at 40° C. The reaction mixture was added dropwise to a stirred aq. NaHCO₃ (1 L) at 0° C. The precipitated were collected by filtration and washed with ice water. The resulting solid was dried under oven lamp to afford 8-bromo-3-chloroisoquinolin-5-ol as a light grey solid (70.0 g, crude). LC-MS (ES, m/z) M+1: 258/260. ¹HNMR (300 MHz, DMSO-d₆) δ 8.82 (s, 1H), 7.99 (s, 1H), 7.33 (d, J=8.6 Hz, 1H), 6.23 (d, J=8.6 Hz, 1H).

Synthesis of 8-bromo-3-chloroisoquinolin-5-yl trifluoromethanesulfonate: Into a 2 L round-bottom flask, were placed 8-bromo-3-chloroisoquinolin-5-ol (70.0 g, 270.8 mmol, 1.0 eq), triethylamine (82.2 g, 812.4 mmol, 3.0 eq) and DCM (700 mL). After that, triflic anhydride (114.6 g, 406.2 mmol, 1.5 eq) was added dropwise at 0° C. and then the resulting mixture was stirred for additional 1 hour at 25° C. The reaction was quenched by the addition of water (500 mL) and extracted with dichloromethane (3×300 mL). The combined organic layers were washed with aq. NaHCO₃ (200 mL), and dried over anhydrous Na₂SO₄. After filtration, the filtrate was concentrated under vacuum. The residue was purified by a flash column (silica gel, ethyl acetate/petroleum ether=1:5) to give 8-bromo-3-chloroisoquinolin-5-yl trifluoromethanesulfonate as a white solid (50.0 g, 47.3%). LC-MS (ES, m/z) M+1: 390/392. ¹HNMR (300 MHz, DMSO-d₆) δ 9.49-9.40 (m, 1H), 8.19 (dt, J=8.3, 1.8 Hz, 1H), 8.00 (ddd, J=8.4, 2.7, 1.6 Hz, 1H), 7.91 (dd, J=4.6, 2.6 Hz, 1H).

### Example INT-3A: Preparation of 2-(4-methoxypiperidin-1-yl)pyrimidin-4-amine

Synthesis of 2-(4-methoxypiperidin-1-yl)pyrimidin-4-amine: Into a 50 mL round-bottom flask, were placed 2-chloropyrimidin-4-amine (1.0 g, 7.7 mmol, 1.0 eq), 4-methoxypiperidine (980 mg, 8.5 mmol, 1.1 eq), triethylamine (2.3 g, 23.2 mmol, 3.0 eq), isopropyl alcohol (16 mL). The resulting mixture was stirred for 16 hours at 80° C. The reaction mixture was concentrated under vacuum and the residue was purified by reverse phase flash using the following conditions (0.1% NH₃·H₂O in acetonitrile, 10% to 18%) to afford 2-(4-methoxypiperidin-1-yl)pyrimidin-4-amine as a white solid (1.4 g, 85.9%). LC-MS (ES, m/z) M+1: 209. ¹HNMR (300 MHz, DMSO-d₆) δ 7.73 (d, J=5.6 Hz, 1H), 6.34 (s, 2H), 5.70 (d, J=5.6 Hz, 1H), 4.17 (dt, J=13.2, 4.6 Hz, 2H), 3.37 (dt, J=8.7, 4.6 Hz, 1H), 3.26 (s, 3H), 3.16 (ddd, J=13.1, 9.7, 3.2 Hz, 2H), 1.89-1.75 (m, 2H), 1.30 (dtd, J=12.9, 9.2, 3.9 Hz, 2H).

### Example INT-4: Preparation of 2-[(3S,4R)-3-fluoro-4-methoxypiperidin-1-yl]pyrimidin-4-amine

Synthesis of tert-butyl (3S,4R)-3-fluoro-4-methoxypiperidine-1-carboxylate: Into a 40 mL round-bottom flask, were placed tert-butyl (3S,4R)-3-fluoro-4-hydroxypiperidine-1-carboxylate (1.0 g, 4.5 mmol, 1.0 eq), THE (10 mL). After that, NaH (218 mg, 5.4 mmol, 1.2 eq) was added in portions at 0° C. The resulting mixture was stirred for 30 min at 0° C. and then methyl iodide (971 mg, 6.8 mmol, 1.5 eq) was added dropwise at 0° C. The resulting mixture was stirred for 3 hours at 25° C. The reaction was quenched with water (10 mL) and extracted with ethyl acetate (2×10 mL). The combined organic layers were washed with brine (10 mL), dried over anhydrous Na₂SO₄. After filtration, the filtrate was concentrated under vacuum to give tert-butyl (3S,4R)-3-fluoro-4-methoxypiperidine-1-carboxylate as light yellow oil (960 mg, 90.2%). ¹HNMR (300 MHz, DMSO-d6) δ 5.01-4.67 (m, 1H), 4.03 (dddd, J=14.7, 9.7, 4.6, 1.9 Hz, 1H), 3.80 (s, 1H), 3.55-3.32 (m, 1H), 3.30 (s, 3H), 3.22-2.78 (m, 2H), 1.77-1.51 (m, 2H), 1.39 (s, 9H).

Synthesis of (3S,4R)-3-fluoro-4-methoxypiperidine hydrochloride salt: Into a 50 mL round-bottom flask, were placed tert-butyl (3S,4R)-3-fluoro-4-methoxypiperidine-1-carboxylate (960 mg, 4.1 mmol, 1.0 eq), DCM (10 mL). To the above mixture was added HCl in dioxane (2 ml) at 25° C. The resulting mixture was stirred for 3 hours at 25° C. The filtrate was concentrated under vacuum to give (3S,4R)-3-fluoro-4-methoxypiperidine hydrochloride salt crude as a yellow salt (800 mg). ¹HNMR (300 MHz, DMSO-d6) δ 5.30-4.91 (m, 1H), 3.73-3.35 (m, 2H), 3.32 (s, 3H), 3.29-3.07 (m, 2H), 2.94 (t, J=12.2 Hz, 1H), 2.01-1.74 (m, 2H).

Synthesis of 2-[(3S,4R)-3-fluoro-4-methoxypiperidin-1-yl]pyrimidin-4-amine: Into a 50 mL round-bottom flask, were placed (3S,4R)-3-fluoro-4-methoxypiperidine (800 mg, 6.0 mmol, 1.0 eq), 2-chloropyrimidin-4-amine (856 mg, 6.6 mmol, 1.1 eq), triethylamine (1.8 g, 18.0 mmol, 3.0 eq), isopropyl alcohol (13 mL). The resulting mixture was stirred for 16 hours at 80° C., and the mixture was concentrated in vacuo. The residue was purified by reverse phase flash using the following conditions (0.1% NH₃·H₂O in acetonitrile, 5% to 25%) to give 2-[(3S,4R)-3-fluoro-4-methoxypiperidin-1-yl]pyrimidin-4-amine as a white solid (560 mg, 41.2%, ee: 99.6%). ¹HNMR (300 MHz, DMSO-d6) δ 7.73 (d, J=5.6 Hz, 1H), 6.39 (t, J=2.5 Hz, 2H), 5.72 (d, J=5.6 Hz, 1H), 5.00-4.70 (m, 1H), 4.55 (dddd, J=14.3, 9.2, 5.2, 1.7 Hz, 1H), 4.36-4.22 (m, 1H), 3.59-3.35 (m, 1H), 3.33 (s, 3H), 3.31-3.19 (m, 1H), 3.10 (tdd, J=10.9, 3.8, 1.4 Hz, 1H), 1.79-1.54 (m, 2H).

### Example 1: Preparation of 1-(3-{8-[(2R,3S)-3-(methanesulfonylmethyl)-2-methylazetidin-1-yl]-3-{[2-(4-methoxypiperidin-1-yl)pyrimidin-4-yl]amino}isoquinolin-5-yl}azetidin-1-yl)prop-2-en-1-one (trans rac)

Synthesis of 3-chloro-5-methoxy-8-[(2R,3S)-2-methyl-3-((methylsulfonyl)methyl)azetidin-1-yl]isoquinoline: Into a 50 mL round-bottom flask purged and maintained with an inert atmosphere of nitrogen, were placed 8-bromo-3-chloro-5-methoxyisoquinoline (600 mg, 2.2 mmol, 1.0 eq), (2R,3S)-3-(methanesulfonylmethyl)-2-methylazetidine trifluoroacetic acid salt (615 mg, 2.2 mmol, 1.0 eq), Cs₂CO₃ (1.4 g, 4.4 mmol, 2.0 eq), 2nd Generation XantPhos precatalyst (195 mg, 0.2 mmol, 0.1 eq), dioxane (10 mL). The resulting mixture was stirred for additional 16 hours at 100° C. The resulting mixture was diluted with water (10 mL) and then extracted with ethyl acetate (2×10 mL). The combined organic layers were washed with brine, dried over anhydrous Na₂SO₄. After filtration, the filtrate was concentrated under vacuum. The crude residue was applied onto a silica gel column and eluted with ethyl acetate/petroleum ether=1:1 to give 3-chloro-5-methoxy-8-[(2R,3S)-2-methyl-3-((methylsulfonyl)methyl)azetidin-1-yl]isoquinoline as a yellow solid (430 mg, 55.0%). LC-MS (ES, m/z) M+1: 355. ¹HNMR (300 MHz, DMSO-d₆) δ 9.12 (d, J=0.9 Hz, 1H), 7.90 (d, J=0.7 Hz, 1H), 7.17 (d, J=8.4 Hz, 1H), 6.75 (d, J=8.4 Hz, 1H), 4.60 (t, J=7.4 Hz, 1H), 4.17 (p, J=6.1 Hz, 1H), 1.39 (d, J=6.0 Hz, 3H) 3.63-3.44 (m, 3H), 3.00 (s, 3H), 2.94-2.81 (m, 1H), 1.39 (d, J=6.0 Hz, 3H).

Synthesis of 3-chloro-8-[(2R,3S)-2-methyl-3-((methylsulfonyl)methyl)azetidin-1-yl]isoquinolin-5-ol: Into a 20 mL round-bottom flask, were placed 3-chloro-5-methoxy-8-[(2R,3S)-2-methyl-3-((methylsulfonyl)methyl)azetidin-1-yl]isoquinoline (430 mg, 1.2 mmol, 1.0 eq), BBr₃ (1 M in DCM, 4 mL). The resulting mixture was stirred for 1 hour at 25° C. The reaction was quenched by the addition of aq. NHCO₃ (5 mL) at 0° C. and then extracted with dichloromethane (3×3 mL). The combined organic layers were dried over anhydrous Na₂SO₄. After filtration, the filtrate was concentrated under vacuum. The crude residue was applied onto a silica gel column and eluted with ethyl acetate/petroleum ether=1:1 to afford 3-chloro-8-[(2R,3S)-2-methyl-3-((methylsulfonyl)methyl)azetidin-1-yl]isoquinolin-5-ol as a yellow solid (152 mg, 36.8%). LC-MS (ES, m/z) M+1: 341. ¹HNMR (300 MHz, DMSO-d₆) δ 9.98 (s, 1H), 9.08 (s, 1H), 7.89 (s, 1H), 7.03 (d, J=8.2 Hz, 1H), 6.70 (d, J=8.3 Hz, 1H), 4.56 (t, J=7.3 Hz, 1H), 4.10 (q, J=6.3 Hz, 1H), 3.61 (q, J=3.4, 2.8 Hz, 1H), 3.62-3.42 (m, 2H), 2.99 (s, 3H), 2.86 (q, J=7.3 Hz, 1H), 1.37 (d, J=5.8 Hz, 3H).

Synthesis of 3-chloro-8-[(2R,3S)-3-(methanesulfonylmethyl)-2-methylazetidin-1-yl]isoquinolin-5-yl trifluoromethanesulfonate: Into an 8 mL sample bottle, were placed 3-chloro-8-[(2R,3S)-3-(methanesulfonylmethyl)-2-methylazetidin-1-yl]isoquinolin-5-ol (170 mg, 0.4 mmol, 1.0 eq), triethylamine (151 mg, 1.4 mmol, 3.0 eq), DCM (2 mL). After that, triflic anhydride (211 mg, 0.7 mmol, 1.5 eq) was added dropwise at 0° C. and the resulting mixture was stirred for additional 2 hours at 25° C. The reaction was quenched by the addition of water (3 mL) and then extracted with dichloromethane (3×3 mL). The combined organic layers were washed with aq. NaHCO₃ (2 mL) and dried over anhydrous Na₂SO₄. After filtration, the filtrate was concentrated under vacuum. The crude residue was applied onto a silica gel column and eluted with ethyl acetate/petroleum ether=1:1 to give 3-chloro-8-[(2R,3S)-3-(methanesulfonylmethyl)-2-methylazetidin-1-yl]isoquinolin-5-yl trifluoromethanesulfonate as a light brown solid (110 mg, 46.6%). LC-MS (ES, m/z) M+1: 473. ¹HNMR (300 MHz, DMSO-d₆) δ 9.23 (s, 1H), 7.77 (d, J=8.8 Hz, 1H), 7.65 (s, 1H), 6.71 (d, J=8.9 Hz, 1H), 4.90 (t, J=8.0 Hz, 1H), 4.38 (p, J=6.0 Hz, 1H), 3.98 (t, J=7.2 Hz, 1H), 3.71-3.44 (m, 2H), 3.01 (s, 3H), 2.95 (dd, J=16.4, 8.6 Hz, 1H), 1.49 (d, J=6.1 Hz, 3H).

Synthesis of (1-(tert-butoxycarbonyl)azetidin-3-yl)zinc(II) iodide: Into an 8 mL sample bottle purged and maintained with an inert atmosphere of nitrogen, were placed Zn (149 mg, 2.3 mmol, 1.3 eq), dry N,N-dimethyl acetamide (2 mL). After that, dibromoethane (50 mg, 0.3 mmol, 0.15 eq) and trimethyl chlorosilane (29 mg, 0.3 mmol, 0.15 eq) were added dropwise at 65° C. After stirring at 65° C. for 30 min, tert-butyl 3-iodoazetidine-1-carboxylate (510 mg, 1.8 mmol, 1.0 eq) was added dropwise to the above mixture and then the resulting mixture was stirred for an additional 3 hours at 65° C. The reaction system was used directly in next step.

Synthesis of 3-{3-chloro-8-[(2R,3S)-3-(methanesulfonylmethyl)-2-methylazetidin-1-yl]isoquinolin-5-yl}azetidine-1-carboxylate: Into an 8 mL sample bottle purged and maintained with an inert atmosphere of nitrogen, were placed 3-chloro-8-[(2R,3S)-3-(methanesulfonylmethyl)-2-methylazetidin-1-yl]isoquinolin-5-yl trifluoromethane-sulfonate (70 mg, 0.1 mmol, 1.0 eq), (1-(tert-butoxycarbonyl)azetidin-3-yl)zinc(II) iodide (in N,N-dimethyl acetamide, 357 uL), CuI (11 mg, 0.06 mmol, 0.4 eq), Pd(dppf)Cl₂ (9 mg, 0.01 mmol, 0.08 eq) and N,N-dimethyl acetamide (1 mL). The resulting mixture was stirred for additional 16 hours at 100° C. The crude residue was applied onto a silica gel column and eluted with ethyl acetate/petroleum ether=1:2 to give tert-butyl 3-{3-chloro-8-[(2R,3S)-3-(methanesulfonyl methyl)-2-methylazetidin-1-yl]isoquinolin-5-yl}azetidine-1-carboxylate as a red solid (53 mg, 74.6%). LC-MS (ES, m/z) M+1: 480.

Synthesis of 3-{3-chloro-8-[(2R,3S)-3-(methanesulfonylmethyl)-2-methylazetidin-1-yl]isoquinolin-5-yl}azetidine-1-carboxylate: Into an 8 mL sample bottle purged and maintained with an inert atmosphere of nitrogen, were placed tert-butyl 3-{3-chloro-8-[(2R,3S)-3-(methanesulfonylmethyl)-2-methylazetidin-1-yl]isoquinolin-5-yl}azetidine-1-carboxylate (58 mg, 0.1 mmol, 1.0 eq), 2-(4-methoxypiperidin-1-yl)pyrimidin-4-amine (28 mg, 0.1 mmol, 1.1 eq), Cs₂CO₃ (44 mg, 0.1 mmol, 1.1 eq), S-phos (5 mg, 0.01 mmol, 0.1 eq), Pd₂(dba)₃·CHCl₃ (13 mg, 0.012 mmol, 0.1 eq), dioxane (1 mL). The resulting mixture was stirred for additional 16 hours at 100° C. The crude residue was applied onto a silica gel column and eluted with ethyl acetate/petroleum ether=1:1 to give tert-butyl 3-{8-[(2R,3S)-3-(methanesulfonylmethyl)-2-methylazetidin-1-yl]-3-{[2-(4-methoxypiperidin-1-yl)pyrimidin-4-yl]amino}isoquinolin-5-yl}azetidine-1-carboxylate as a brown solid (40 mg, 50.8%). LC-MS (ES, m/z) M+1: 652.

Synthesis of 5-(azetidin-3-yl)-8-[(2R,3S)-3-(methanesulfonylmethyl)-2-methylazetidin-1-yl]-N-[2-(4-methoxypiperidin-1-yl)pyrimidin-4-yl]isoquinolin-3-amine: Into an 8 mL sample bottle, were placed tert-butyl 3-{8-[(2R,3S)-3-(methanesulfonylmethyl)-2-methylazetidin-1-yl]-3-{[2-(4-methoxypiperidin-1-yl)pyrimidin-4-yl]amino}isoquinolin-5-yl}azetidine-1-carboxylate (40 mg, 0.06 mmol, 1.0 eq), trifluoroacetic acid (0.1 mL) and DCM (1 mL). The resulting mixture was stirred for additional 3 hours at 25° C. The reaction was quenched by the addition of aq. NaHCO₃ (1 mL) and extracted with dichloromethane (2×2 mL). The combined organic layers were dried over anhydrous Na₂SO₄. After filtration, the filtrate was concentrated under vacuum to give 5-(azetidin-3-yl)-8-[(2R,3S)-3-(methanesulfonylmethyl)-2-methyl azetidin-1-yl]-N-[2-(4-methoxypiperidin-1-yl)pyrimidin-4-yl]isoquinolin-3-amine as a red oil (25 mg, 73.8%). LC-MS (ES, m/z) M+1: 552.

Synthesis of 1-(3-{8-[(2R,3S)-3-(methanesulfonylmethyl)-2-methylazetidin-1-yl]-3-{[2-(4-methoxypiperidin-1-yl)pyrimidin-4-yl]amino}isoquinolin-5-yl}azetidin-1-yl)prop-2-en-1-one: Into an 8 mL sample bottle, were placed 5-(azetidin-3-yl)-8-[(2R,3S)-3-(methanesulfonylmethyl)-2-methylazetidin-1-yl]-N-[2-(4-methoxypiperidin-1-yl)pyrimidin-4-yl]isoquinolin-3-amine (25 mg, 0.05 mmol, 1.0 eq), triethylamine (14 mg, 0.14 mmol, 3.0 eq), DCM (1 mL). After that, acryloyl chloride (6 mg, 0.07 mmol, 1.5 eq) was added dropwise at 0° C. The resulting mixture was stirred for additional 2 hours at 25° C. The reaction was quenched by the addition of aq. NaHCO₃ (1 mL) and then extracted with dichloromethane (2×2 mL). The combined organic layers were dried over anhydrous Na₂SO₄. After filtration, the filtrate was concentrated under vacuum and the crude residue was applied onto a silica gel column and eluted with ethyl acetate/petroleum ether=l:1 to give 1-(3-{8-[(2R,3S)-3-(methanesulfonylmethyl)-2-methylazetidin-1-yl]-3-{[2-(4-methoxypiperidin-1-yl)pyrimidin-4-yl]amino}isoquinolin-5-yl}azetidin-1-yl)prop-2-en-1-one as a yellow solid (3 mg, 14.6%). LC-MS (ES, m/z) M+1: 606. ¹HNMR (300 MHz, Chloroform-d) δ 9.17 (s, 1H), 8.13 (d, J=6.6 Hz, 1H), 7.59 (d, J=7.6 Hz, 1H), 6.64 (d, J=8.0 Hz, 1H), 6.47-6.35 (m, 1H), 6.26 (dd, J=17.0, 10.1 Hz, 1H), 5.74 (d, J=10.0 Hz, 1H), 5.37 (t, J=4.9 Hz, 1H), 4.77 (t, J=7.6 Hz, 2H), 4.59 (s, 1H), 4.38 (d, J=5.8 Hz, 1H), 4.34 (s, 3H), 4.11 (s, 5H), 3.82 (t, J=7.1 Hz, 1H), 3.61 (s, 1H), 3.46 (dd, J=13.6, 7.3 Hz, 1H), 3.40 (s, 3H), 3.11 (d, J=7.1 Hz, 1H), 3.02 (s, 3H), 2.00 (s, 3H), 1.90 (s, 3H), 1.55 (d, J=6.1 Hz, 3H).

### Example 2: Preparation of 1-[(2S)-2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidin-1-yl]prop-2-en-1-one (assumed)

Synthesis of 8-bromo-3-chloroisoquinolin-5-ol: Into a 2 L round-bottom flask, were placed 8-bromo-3-chloro-5-methoxyisoquinoline (70.0 g, 256.9 mmol, 1 eq), BBr₃ (1 M in DCM, 700 mL). The resulting mixture was stirred for 2 days at 40° C. The reaction mixture was quenched with aq. NaHCO₃ (1.0 L) at 0° C. The precipitated solids were collected by filtration and washed with ice water. The resulting solid was dried under oven lamp to give 8-bromo-3-chloroisoquinolin-5-ol crude as a light grey solid (70.0 g). ¹HNMR (300 MHz, DMSO-d₆) δ 8.82 (s, 1H), 7.99 (s, 1H), 7.33 (d, J=8.6 Hz, 1H), 6.23 (d, J=8.6 Hz, 1H).

Synthesis of 8-bromo-3-chloro-5-{[2-(trimethylsilyl)ethoxy]methoxy}isoquinoline: Into a 250 mL round-bottom flask, were placed 8-bromo-3-chloro-4a,8a-dihydroisoquinolin-5-ol (3.5 g, 13.4 mmol, 1.0 eq), cesium carbonate (13.1 g, 40.3 mmol, 3.0 eq), DMF (70 mL). After that, (2-(chloromethoxy)ethyl)trimethylsilane (3.36 g, 20.1 mmol, 1.5 eq) was added at 25° C. The resulting mixture was stirred for 16 hours at 25° C. The reaction was quenched with water (100 mL) and then extracted with dichloromethane (2×100 mL). The combined organic layers were dried over anhydrous Na₂SO₄. After filtration, the filtrate was concentrated under vacuum. The crude residue was purified onto a silica gel column and eluted with petroleum ether/ethyl acetate=5:1 to give 8-bromo-3-chloro-5-{[2-(trimethylsilyl)ethoxy]methoxy}isoquinoline as a grey solid (4.5 g, 86.1%). ¹HNMR (300 MHz, DMSO-d6) δ 9.22 (d, J=0.9 Hz, 1H), 8.00 (dd, J=10.2, 0.8 Hz, 1H), 7.91 (d, J=8.3 Hz, 1H), 7.32 (d, J=8.4 Hz, 1H), 5.49 (s, 2H), 3.84-3.72 (m, 2H), 0.95-0.83 (m, 2H), −0.05 (s, 9H).

Synthesis of 3-chloro-8-[(2R,3S)-3-(methanesulfonylmethyl)-2-methylazetidin-1-yl]-5-{[2-(trimethylsilyl)ethoxy]methoxy}isoquinoline (trans rac): Into a 250 mL round-bottom flask purged and maintained with an inert atmosphere of nitrogen, were placed 8-bromo-3-chloro-5-{[2-(trimethylsilyl)ethoxy]methoxy}isoquinoline (4.5 g, 11.5 mmol, 1.0 eq), (2R,3S)-3-(methanesulfonylmethyl)-2-methylazetidine trifluoroacetic acid salt (trans rac) (1.9 g, 11.5 mmol, 1.0 eq), Cs₂CO₃ (7.5 g, 23.1 mmol, 2.0 eq), 2nd Generation XantPhos precatalyst (1.0 g, 1.1 mmol, 0.1 eq), dioxane (90 mL). The resulting mixture was stirred for 16 hours at 100° C. The reaction mixture was diluted with water (100 mL) and extracted with ethyl acetate (2×80 mL). The combined organic layers were washed with brine, dried over anhydrous Na₂SO₄. After filtration, the filtrate was concentrated under vacuum. The crude residue was purified onto a silica gel column and eluted with petroleum ether/ethyl acetate=4:1 to give 3-chloro-8-[(2R,3S)-3-(methanesulfonylmethyl)-2-methylazetidin-1-yl]-5-{[2-(trimethylsilyl)ethoxy]methoxy}isoquinoline (trans rac) as a yellow solid (2.3 g, 42.1%). ¹HNMR (300 MHz, DMSO-d₆) δ 9.11 (s, 1H), 7.89 (s, 1H), 7.32 (d, J=8.4 Hz, 1H), 6.73 (d, J=8.5 Hz, 1H), 5.35 (s, 2H), 4.63 (t, J=7.5 Hz, 1H), 4.20 (q, J=6.2 Hz, 1H), 3.82-3.71 (m, 2H), 3.56 (dt, J=17.0, 7.0 Hz, 3H), 2.99 (s, 3H), 2.89 (p, J=6.9, 6.5 Hz, 1H), 1.39 (d, J=6.0 Hz, 3H), 0.95-0.77 (m, 2H), −0.03 (s, 9H).

Synthesis of 3-chloro-8-[(2R,3S)-3-(methanesulfonylmethyl)-2-methylazetidin-1-yl]isoquinolin-5ol-(trans rac): Into a 50 mL round-bottom flask, were placed 3-chloro-8-[(2R,3S)-3-(methanesulfonylmethyl)-2-methylazetidin-1-yl]-5-{[2-(trimethylsilyl)ethoxy]methoxy}isoquinoline (trans rac) (2.0 g, 4.2 mmol, 1.0 eq), CF₃COOH (4 mL), DCM (20 mL). The resulting mixture was stirred for 1 hour at 25° C. The reaction was quenched with aq. NaHCO₃ (10 mL) and extracted with dichloromethane (3×10 mL). The combined organic layers were dried over anhydrous Na₂SO₄. After filtration, the filtrate was concentrated under vacuum. The crude residue was purified onto a silica gel column and eluted with petroleum ether/ethyl acetate=1:1 to give 3-chloro-8-[(2R,3S)-3-(methanesulfonylmethyl)-2-methylazetidin-1-yl]isoquinolin-5-ol (trans rac) as a yellow solid (1.4 g, 98.1%). ¹HNMR (300 MHz, DMSO-d6) δ 9.98 (s, 1H), 9.08 (d, J=0.8 Hz, 1H), 7.89 (d, J=0.7 Hz, 1H), 7.03 (d, J=8.2 Hz, 1H), 6.70 (d, J=8.3 Hz, 1H), 4.56 (t, J=7.3 Hz, 1H), 4.12 (p, J=5.9 Hz, 1H), 3.51 (td, J=8.9, 8.0, 5.8 Hz, 3H), 2.99 (s, 3H), 2.84 (dt, J=15.0, 7.5 Hz, 1H), 1.37 (d, J=6.1 Hz, 3H).

Synthesis of 3-chloro-8-[(2R,3S)-3-(methanesulfonylmethyl)-2-methylazetidin-1-yl]isoquinolin-5ol-(assumed): The crude product of 3-chloro-8-[(2R,3S)-3-(methanesulfonylmethyl)-2-methylazetidin-1-yl]isoquinolin-5-ol (1 g, trans rac) was purified by Prep-SFC using the following conditions. Finally, 3-chloro-8-[(2S,3R)-3-(methanesulfonylmethyl)-2-methylazetidin-1-yl]isoquinolin-5-ol (assumed) (300 mg, 30.0%, ee: 100%, 5A) and 3-chloro-8-[(2R,3S)-3-(methanesulfonylmethyl)-2-methylazetidin-1-yl]isoquinolin-5-ol (assumed) (330 mg, 33.0%, ee: 98.4%, 5B) as a yellow solid. 5A, TR=4.2 min in CHIRAL-SFC, Column: Cellulose SA 50*3.0 mm, 3.0 um; Mobile Phase A: CO2, Mobile Phase B: MeOH: DCM=2:1; Flow rate: 80 mL/min; Gradient: isocratic 35% B; Wave Length: 220 nm. 5B, TR=7 min in CHIRAL-SFC, Column: Cellulose SA 50*3.0 mm, 3.0 um; Mobile Phase A: CO2, Mobile Phase B: MeOH: DCM=2:1; Flow rate: 80 mL/min; Gradient: isocratic 35% B; Wave Length: 220 nm. LC-MS (ES, m/z) M+1:341.

Synthesis of 3-chloro-8-[(2R,3S)-3-(methanesulfonylmethyl)-2-methylazetidin-1-yl]isoquinolin-5-yl trifluoromethanesulfonate (assumed): Into a 50 mL round-bottom flask, were placed 3-chloro-8-[(2R,3S)-3-(methanesulfonylmethyl)-2-methylazetidin-1-yl]isoquinolin-5-ol (assumed) (470 mg, 1.3 mmol, 1.0 eq), triethylamine (418 mg, 4.1 mmol, 3.0 eq), DCM (10 mL). After that, triflic anhydride (583 mg, 2.0 mmol, 1.5 eq) was added dropwise at 0° C. and then stirred for 1 hour at 25° C. The reaction was quenched with water (5 mL) and extracted with dichloromethane (3×5 mL). The combined organic layers were washed with aq. NaHCO₃ (10 mL), and dried over anhydrous Na₂SO₄. After filtration, the filtrate was concentrated under vacuum. The residue was purified onto a silica gel column and eluted with petroleum ether/ethyl acetate=2:1 to give 3-chloro-8-[(2R,3S)-3-(methanesulfonylmethyl)-2-methylazetidin-1-yl]isoquinolin-5-yl trifluoromethanesulfonate (assumed) as a grey solid (400 mg, 61.3%). ¹HNMR (300 MHz, Chloroform-d) δ 9.17 (d, J=0.8 Hz, 1H), 7.79 (d, J=0.8 Hz, 1H), 7.53 (d, J=8.6 Hz, 1H), 6.54 (d, J=8.8 Hz, 1H), 4.81 (t, J=8.0 Hz, 1H), 4.42 (p, J=6.1 Hz, 1H), 3.91 (dd, J=7.9, 6.4 Hz, 1H), 3.50-3.32 (m, 2H), 3.13 (h, J=7.1 Hz, 1H), 3.00 (s, 3H), 1.59 (d, J=6.2 Hz, 3H).

Synthesis of tert-butyl 2-{3-chloro-8-[(2R,3S)-3-(methanesulfonylmethyl)-2-methylazetidin-1-yl]isoquinolin-5-yl}-4,5-dihydropyrrole-1-carboxylate (assumed): Into an 8 mL sample bottle purged and maintained with an inert atmosphere of nitrogen, were placed 3-chloro-8-[(2R,3S)-3-(methanesulfonylmethyl)-2-methylazetidin-1-yl]isoquinolin-5-yl trifluoromethanesulfonate (assumed) (380 mg, 0.8 mmol, 1.0 eq), tert-butyl 2,3-dihydropyrrole-1-carboxylate (271 mg, 1.6 mmol, 2.0 eq), Pd₂(dba)₃·CHCl₃ (166 mg, 0.16 mmol, 0.2 eq), X-Phos (77 mg, 0.16 mmol, 0.2 eq), triethylamine (0.75 mL), acetonitrile (0.75 mL). The resulting mixture was stirred for 16 hours at 70° C., and then the mixture was concentrated in vacuo. The residue was purified onto a silica gel column and eluted with petroleum ether/ethyl acetate=1:1 to give tert-butyl 2-{3-chloro-8-[(2R,3S)-3-(methanesulfonylmethyl)-2-methylazetidin-1-yl]isoquinolin-5-yl}-4,5-dihydropyrrole-1-carboxylate (assumed) as a grey solid (180 mg, 31.1%). LC-MS (ES, m/z) M+1: 492.

Synthesis of tert-butyl 2-{3-chloro-8-[(2R,3S)-3-(methanesulfonylmethyl)-2-methylazetidin-1-yl]isoquinolin-5-yl}pyrrolidine-1-carboxylate (assumed): Into a 50 mL round-bottom flask, were placed tert-butyl 2-{3-chloro-8-[(2R,3S)-3-(methanesulfonylmethyl)-2-methylazetidin-1-yl]isoquinolin-5-yl}-4,5-dihydropyrrole-1-carboxylate (assumed) (180 mg, 0.2 mmol, 1.0 eq, 68.4%), PtO₂ (63 mg), ethyl acetate (10 mL). The resulting mixture was stirred for 30 mins at 25° C. under atmosphere of H₂ balloon. The solid was filtered out and the filtrate was concentrated under vacuum. The residue was purified by reverse phase flash using the following conditions (0.1% NH₃·H₂O in acetonitrile, 10% to 70%) to give tert-butyl 2-{3-chloro-8-[(2R,3S)-3-(methanesulfonylmethyl)-2-methylazetidin-1-yl]isoquinolin-5-yl}pyrrolidine-1-carboxylate (assumed) as a yellow solid (120 mg, 97.1%). LC-MS (ES, m/z) M+1: 494.

Synthesis of tert-butyl 2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidine-1-carboxylate (assumed): Into an 8 mL sample bottle purged and maintained with an inert atmosphere of nitrogen, were placed tert-butyl 2-{3-chloro-8-[(2R,3S)-3-(methanesulfonylmethyl)-2-methylazetidin-1-yl]isoquinolin-5-yl}pyrrolidine-1-carboxylate (assumed) (140 mg, 0.283 mmol, 1.0 eq), 2-[(3S,4R)-3-fluoro-4-methoxypiperidin-1-yl]pyrimidin-4-amine (70 mg, 0.311 mmol, 1.1 eq), Pd₂(dba)₃·CHCl₃ (29.33 mg, 0.028 mmol, 0.1 eq), CS₂CO₃ (101 mg, 0.311 mmol, 1.1 eq), S-Phos (11 mg, 0.028 mmol, 0.1 eq), dioxane (2 mL). The resulting mixture was stirred for 16 hours at 100° C. The residue was purified onto a silica gel column and eluted with petroleum ether/ethyl acetate=1:1 to give tert-butyl 2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidine-1-carboxylate (assumed) as a yellow solid (98 mg, 50.5%). LC-MS (ES, m/z) M+1: 684.

Synthesis of N-{2-[(3S,4R)-3-fluoro-4-methoxypiperidin-1-yl]pyrimidin-4-yl}-8-[(2R,3S)-3-(methanesulfonylmethyl)-2-methylazetidin-1-yl]-5-(pyrrolidin-2-yl)isoquinolin-3-amine (assumed): Into an 8 mL sample bottle, were placed tert-butyl 2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidine-1-carboxylate (assumed) (98 mg, 0.143 mmol, 1.0 eq), trifluoroacetic acid (0.2 mL), DCM (1 mL). The resulting mixture was stirred for 2 hours at 25° C. The reaction was quenched with aq. NaHCO₃ (1 mL) and extracted with dichloromethane (2×2 mL). The combined organic layers were dried over anhydrous Na₂SO₄. After filtration, the filtrate was concentrated under vacuum to give N-{2-[(3S,4R)-3-fluoro-4-methoxypiperidin-1-yl]pyrimidin-4-yl}-8-[(2R,3S)-3-(methanesulfonylmethyl)-2-methylazetidin-1-yl]-5-(pyrrolidin-2-yl)isoquinolin-3-amine (assumed) as light solid (70 mg, 83.6%). LC-MS (ES, m/z) M+1: 584.

Synthesis of 1-{2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidin-1-yl}prop-2-en-1-one (assumed): Into an 8 mL sample bottle, were placed N-{2-[(3S,4R)-3-fluoro-4-methoxypiperidin-1-yl]pyrimidin-4-yl}-8-[(2R,3S)-3-(methanesulfonylmethyl)-2-methylazetidin-1-yl]-5-(pyrrolidin-2-yl)isoquinolin-3-amine (assumed) (70 mg, 0.120 mmol, 1.0 eq), triethylamine (36 mg, 0.360 mmol, 3.0 eq), DCM (1 mL). After that, acryloyl chloride (10 mg, 0.120 mmol, 1.0 eq) was added at 0° C. The resulting mixture was stirred for 2 hours at 25° C. The reaction was quenched with aq. NaHCO₃ (1 mL) and then extracted with dichloromethane (2×1 mL). The combined organic layers were dried over anhydrous Na₂SO₄. After filtration, the filtrate was concentrated under vacuum and the residue was purified by reverse phase flash using the following conditions (0.1% NH₃·H₂O in acetonitrile, 10% to 60%) to give 1-{2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidin-1-yl}prop-2-en-1-one (assumed) as a yellow solid (55 mg, 71.2%). LC-MS (ES, m/z) M+1: 638.

Synthesis of 1-[(2S)-2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidin-1-yl]prop-2-en-1-one (assumed): The crude product of N-{2-[(3S,4R)-3-fluoro-4-methoxypiperidin-1-yl]pyrimidin-4-yl}-8-[(2R,3S)-3-(methanesulfonylmethyl)-2-methylazetidin-1-yl]-5-(pyrrolidin-2-yl)isoquinolin-3-amine (55 mg) was purified by Prep-SFC using the following conditions. Finally, 1-[(2S)-2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidin-1-yl]prop-2-en-1-one (assumed) (17 mg, 30.9%, ee: 100%) was obtained as a yellow solid. TR=7.2 min in CHIRAL-SFC, Column: CHIRAL ART Cellulose-SZ, 3*25 cm, 5 um; Mobile Phase A: CO2, Mobile Phase B: MeOH: DCM=2: 1; Flow rate: 80 mL/min; Gradient: isocratic 45% B; Wave Length: 220 nm. LC-MS (ES, m/z) M+1: 638. ¹HNMR (300 MHz, DMSO-d6) δ 10.23 (s, 1H), 9.12 (s, 1H), 8.45 (d, J=5.9 Hz, 1H), 8.02 (dd, J=5.9, 3.7 Hz, 1H), 7.14 (dd, J=8.0, 5.4 Hz, 1H), 6.77 (dd, J=16.8, 10.3 Hz, 1H), 6.54 (td, J=10.6, 6.9 Hz, 2H), 6.18-0.94 (m, 2H), 5.77-5.65 (m, 1H), 5.59 (d, J=7.5 Hz, 1H), 5.45 (dd, J=9.4, 3.4 Hz, 1H), 4.91 (d, J=50.0 Hz, 1H), 4.72 (q, J=7.1 Hz, 2H), 4.47 (s, 1H), 4.21 (q, J=6.2 Hz, 1H), 4.03 (t, J=9.1 Hz, 1H), 3.91-3.42 (m, 7H), 3.00 (s, 3H), 2.90 (q, J=7.3 Hz, 1H), 2.49-2.09 (m, 1H), 2.08-1.54 (m, 6H), 1.44 (dd, J=6.1, 2.0 Hz, 3H), 1.21 (d, J=16.9 Hz, 1H).

### Example 3: Preparation of 1-[(2R)-2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidin-1-yl]prop-2-en-1-one (assumed)

Synthesis of 1-[(2R)-2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidin-1-yl]prop-2-en-1-one (assumed): The crude product of N-{2-[(3S,4R)-3-fluoro-4-methoxypiperidin-1-yl]pyrimidin-4-yl}-8-[(2R,3S)-3-(methanesulfonylmethyl)-2-methylazetidin-1-yl]-5-(pyrrolidin-2-yl)isoquinolin-3-amine (55 mg) was purified by Prep-SFC with the following conditions. Finally, 1-[(2R)-2-[3-({2-[(3S,4R)-3-fluoro-4-methoxypiperidin-1-yl]pyrimidin-4-yl}amino)-8-[(2R,3S)-3-(methanesulfonylmethyl)-2-methylazetidin-1-yl]isoquinolin-5-yl]pyrrolidin-1-yl]prop-2-en-1-one (assumed) (17 mg, 30.9%, ee: 99.82%) as a yellow solid. TR=10.5 min in CHIRAL-SFC, Column: CHIRAL ART Cellulose-SZ, 3*25 cm, 5 um; Mobile Phase A: CO2, Mobile Phase B: MeOH: DCM=2:1; Flow rate: 80 mL/min; Gradient: isocratic 45% B; Wave Length: 220 nm. LC-MS (ES, m/z) M+1: 638. ¹HNMR (300 MHz, DMSO-d6) δ 10.23 (s, 1H), 9.12 (s, 1H), 8.45 (d, J=5.9 Hz, 1H), 8.02 (dd, J=5.9, 3.7 Hz, 1H), 7.14 (dd, J=8.0, 5.4 Hz, 1H), 6.77 (dd, J=16.8, 10.3 Hz, 1H), 6.54 (td, J=10.6, 6.9 Hz, 2H), 6.18-0.94 (m, 2H), 5.77-5.65 (m, 1H), 5.59 (d, J=7.5 Hz, 1H), 5.45 (dd, J=9.4, 3.4 Hz, 1H), 4.91 (d, J=50.0 Hz, 1H), 4.72 (q, J=7.1 Hz, 2H), 4.47 (s, 1H), 4.21 (q, J=6.2 Hz, 1H), 4.03 (t, J=9.1 Hz, 1H), 3.91-3.42 (m, 7H), 3.00 (s, 3H), 2.90 (q, J=7.3 Hz, 1H), 2.49-2.09 (m, 1H), 2.08-1.54 (m, 6H), 1.44 (dd, J=6.1, 2.0 Hz, 3H), 1.21 (d, J=16.9 Hz, 1H).

### Biological Example 1. Biochemical EGFR Inhibition Assays

Inhibitory effects of the compounds of the disclosure were measured in biochemical assays that measure the phosphorylation activity of EGFR enzyme phosphorylates 2.5 micromolar 5-FAM-EEPLYWSFPAKKK-CONFF peptide substrate (FL-Peptide 22, PerkinElmer, 760366) in the presence of adenosine-5′-triphosphate (ATP) and varying concentrations of the test compound in 100 mM 2-[4-(2-hydroxyethyl)piperazin-1-yl]ethanesulfonic acid (HEPES), pH 7.5, 10 mM MgCE. 0.015% Brij-35, 1 mM dithiothreitol (DTT), 1.0% dimehylsulfoxide (DMSO). Assays were performed at 1.0 mM ATP or at ATP Km of the EGFR enzymes. Reactions proceeded until between 10% to 20% total peptides were phosphorylated at room temperature (25° C.) and were terminated with 35 mM 2,2′,2″,2″-(ethane-1,2-diyldinitrilo)tetraacetic acid (EDTA). Product was detected using the Caliper mobility shift detection method where the phosphorylated peptide (product) and substrate were electrophoretically separated and measured. Percent activity was plotted against log concentration of compound and points to generate an apparent IC50. The following enzyme forms of EGFR were examples that were used in these assays: EGFR L858R (SignalChem, E10-122BG); EGFR (d746-750) (SignalChem, E10-122JG); EGFR (L858R T790M C797S) (SignalChem, E10-122VG); EGFR (d746-750) T790M C797S (SignalChem, E10-122UG); The following table lists the IC50 values of certain compounds of the invention.

| | | | | L858R | d746-750 |
|---|---|---|---|---|---|
| | | L858R | d746-750 | T790M | T790M |
| Example | L858R | T790M | T790M | C797S | C797S |
| 2 | 100 nM | | | | 2.3 nM |
| 3 | 1.5 nM | 0.29 nM | 0.88 nM | 0.69 nM | 0.15 nM |

### Biological Example 2: EGFR In Vitro Covalent MS-Based Assay

EGFR proteins at 2 uM final concentration were incubated with the test compounds at the doses and time points indicated in a buffer containing 20 mmol/L HEPES pH 7.5, 150 mmol/L NaCl, 1 mmol/L MgCl 2, and 1 mmol/L DTT. Reactions were quenched by adding formic acid to 0.2%. Samples were buffer exchanged and analyzed by denaturing SEC-MS (Orbitrap Eclipse), Mobile phase: 30% ACN, 0.1% formic acid, 0.02% TFA. Raw data were analyzed by charge state deconvolution with BioPharma Finder 4.1 Intact Mass, ReSpect and Sliding Window algorithms. The masses were calculated via software for EGFR theoretical sequence and experimental compounds.

### Biological Example 3. NCI-H1975 pEGFR AlphaLISA Assays

Inhibitory effects of the compounds of the disclosure were evaluated in cellular assays that measure level of intracellular phosphorylation of EGFR in NCI-H1975 cell line that harbors the EGFR L858R T790M mutations (ATCC, CRL-5908) using AlphaLISA sureFire ultra p-EGFR (Tyrl068) assay kit (PerkinElmer, ALSU-PEGFR-A50K). The NCI-H1975 cells were seeded at 12.5K/well in 22 pL into 384 well opti plate (PerkinElmer, 6007299) and adhering overnight at 37C/5% CO2. On the next day, the test compounds and DMSO control were added into H 1975 cell plate followed by incubation at 37C/5% CO2 for 4-5 hours. The cells were then spin down in the 384-well plate and lysed with 10pL of Ix AlphaLISA lysis buffer followed by shaking at 600 rpm for 10 minutes at room temperature. After that, 5 μL of an acceptor bead mix was added to each well followed by incubation at room temperature for 1.5-2 h in dark. Then 5pL of a donor bead mix was added to each well followed by overnight incubation at room temperature in dark. On the next day, the plate was read at a compatible plate reader to obtain pEGFR signal. Percent of pEGFR inhibition was plotted against log concentration of compounds to generate IC50 values.

### Biological Example 4: EGFR Degradation Assay (Cellular)

Generation of BaF3 EGFR mutant cell lines: The BaF3 parental line was purchased from DSMZ and grown in RPMI media supplemented with 10% FBS and 10 ng/mL interleukin 3 (IL-3) (Thermo Fisher Scientific). EGFR mutants (T790M/L853R, T790M/L853R/C797S) were cloned into the pCDH lentiviral vector (SystemBio) under the control of a PGK promoter and confirmed by DNA sequencing. The resulting gene expression vector for each mutant was mixed with packaging vectors and cotransfected into 2×10E6 HEK293T cells (ATCC) in 10 mL of DMEM media to generate lentiviral particles according to the manufacturers protocol (Origene). Three days post-transfection, the viral supernatant was harvested and filtered. In one well of a 12-well plate, 0.5 mL of viral supernatant was added to 2E6 Ba/F3 cells contained in 1.5 mL of RPMI media including 10% FBS, 10 ng/mL IL-3, and 5 μg/mL polybrene (Invitrogen). The plate was centrifuged at 2,000 rpm for 1 hour at room temperature and infected cells were kept in a tissue culture incubator overnight at 37° C. The cells were washed once in fresh BaF3 media and reseeded at 0.5E6 cells/well of a 12-well plate in media supplemented with 0.5 pg/mL puromycin. The cells were maintained in this media for 3 weeks. IL-3-independent, EGFR mutant transformed cells were routinely maintained in RPMI medium supplemented with 10% FBS. RPMI 1640 no-phenol red medium and fetal bovine serum (FBS) were purchased from Gibco (Grand Island, NY, USA). EGFR total kit and EGFR phospho-Y1068 kit were purchased from Cisbio (Bedford, MA, USA). BaF3 EGFR mutant cell lines (EGFR T790M/L858R/C797S) cell line was generated in house, according to the protocol reported above. Cell culture flasks and 384-well microplates were acquired from VWR (Radnor, PA, USA).

EGFR degradation analysis: EGFR degradation was determined based on quantification of FRET signal using EGFR total kit. The FRET signal detected correlates with total EGFR protein level in cells. Briefly, test compounds were added to the 384-well plate from a top concentration of 1 mM with 11 points, half log titration in quadruplicates. Then, BaF3 EGFR mutant cell lines (EGFR T790M/L858R/C797S) were added into 384-well plates at a cell density of 10000 cells per well. The plates were kept at 37° C. with 5% CO2 for 4 hours. After 4-hour incubation, 4× lysis buffer was added to the cells, and then then microplate was agitated on plate shaker at 500 rpm for 30 minutes at room temperature. Next, total EGFR antibody solution was added to the cells and the cells were incubated for another 4 hours at room temperature. Finally, FRET signal was acquired on EnVision™ Multilabel Reader (PerkinElmer, Santa Clara, CA, USA). The cells treated in the absence of the test compound were the negative control and lysis buffer with antibody solution only were the positive control.

### Biological Example 5: In Vitro Anti-Proliferation Assay in Cancer Cell Line with EGFR Mutation

Cell antiproliferation was assayed by PerkinElmer ATPlite™ Luminescence Assay System. Briefly, the various test cancer cell lines were plated at a density of about 1×10⁴ cells per well in Costar 96-well plates, and were incubated with different concentrations of compounds for about 72 hours in medium supplemented with 5% FBS or 10% normal human serum(NHS). One lyophilized substrate solution vial was then reconstituted by adding 5 mL of substrate buffer solution, and was agitated gently until the solution was homogeneous. About 50 μL of mammalian cell lysis solution was added to 100 μL of cell suspension per well of a microplate, and the plate was shaken for about five minutes in an orbital shaker at ˜700 rpm. This procedure was used to lyse the cells and to stabilize the ATP. Next, 50 pL substrate solution was added to the wells and microplate was shaken for five minutes in an orbital shaker at ˜700 rpm. Finally, the luminescence was measured by a PerkinElmer TopCount® Microplate Scintillation Counter. Such assays, carried out with a range of doses of test compounds, allowed the determination of the cellular anti-anti proliferative IC₅₀ of the compounds of the present invention.

### Biological Example 6: Mice PK Study

The pharmacokinetics of compounds were evaluated in CD-1 mouse via Intravenous and Oral Administration. The IV dose was administered as a slow bolus in the Jugular vein, and oral doses were administered by gavage. The formulation for IV dosing was 5% DMSO in 20% HPBCD in water, and the PO formulation was 2.5% DMSO, 10% EtOH, 20% Cremphor EL, 67.5% D5W. The PK time point for the IV arm was 5, 15, 30 min, 1, 2, 4, 6, 8, 12, 24 hours post dose, and for PO arm was 15, 30 min, 1, 2, 4, 6, 8, 12, 24 hours post dose. Approximately 0.03 mL blood was collected at each time point. Blood of each sample was transferred into plastic micro centrifuge tubes containing EDTA-K2 and collect plasma within 15 min by centrifugation at 4000 g for 5 minutes in a 4° C. centrifuge. Plasma samples were stored in polypropylene tubes. The samples were stored in a freezer at−75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary.

### Biological Example 7: In Vivo Xenograft Studies

Typically, athymic nude mice (CD-1 nu/nu) or SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach˜100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/T0=100×ΔT/T0. Values of <42% are considered significant.

What is claimed is:

1. A compound of Formula (1), or an N-oxide thereof, or a pharmaceutically acceptable salt, solvate, polymorph, tautomer, stereoisomer, an isotopic form, or a prodrug of said compound of Formula (1) or N-oxide thereof: 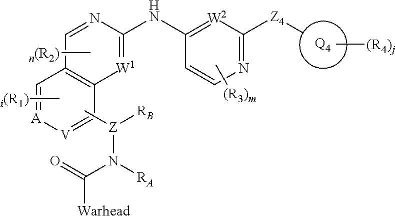
wherein
Warhead is chemical group that can forms a covalent bond with the thiol group of Cys797 in EGFR;
W¹ is CH and W² is N; or W¹ is N and W² is CH;
each of A and V, independently, is N or CH; wherein R1 or Z can be linked to A or Z when A or Z is CH;
Q₄, is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{d};
Z is absent, alkyl, alkenyl, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, or bridged-heterocyclic, each of which is independently optionally substituted with one or more R_{d};
Z₄ is a bond, (CRₐR_{b})ₚ, N(Rₐ), O, S, C(O), S(O₂), —O(CRₐR_{b})ₚ, —N(Rₐ)(CRₐR_{b})ₚ, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, (CRₐR_{b})ₚC(O)N(Rₐ)(CRₐR_{b})_{q}, a bivalent alkenyl group, or a bivalent alkynyl group;
each of R_{A}, R_{B}, R₁, R₂, R₃, or R₄, independently, is absent, H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{d};
Rₐ, R_{b}, R_{c} and R_{d}, independently, is H, D, alkyl, alkenyl, alkynyl, spiroalkyl, halo, cyano, amine, nitro, hydroxy, ═O, -alkyl-O—P(O)(OH)(OH), C(O)NHOH, C(O)OH, C(O)NH₂, —SO₂Rₐ, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more Rₐ;
Rₑ is H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, —SO₂R_{f}, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{f};
R_{f} is H, D, alkyl, alkenyl, alkynyl, spiroalkyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of which is independently optionally substituted with one or more R_{g};
R_{g} is H, D, alkyl, alkenyl, alkynyl, spiroalkyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
R_{A} and R_{B} groups, taken together with the atom to which they are attached, may optionally form a heterocycloalkyl optionally substituted with one or more R_{d};
two of R₁ groups, taken together with the atom to which they are attached, may optionally form a cycloalkyl or heterocycloalkyl optionally substituted with one or more R_{d};
two of R₂ groups, taken together with the atom to which they are attached, may optionally form a cycloalkyl or heterocycloalkyl optionally substituted with one or more R_{d};
two of R₃ groups, taken together with the atom to which they are attached, may optionally form a cycloalkyl or heterocycloalkyl optionally substituted with one or more R_{d};
two of R_{d} groups, taken together with the atom to which they are attached, may optionally form a cycloalkyl or heterocycloalkyl optionally substituted with one or more Rₑ;
two of Rₑ groups, taken together with the atom to which they are attached, may optionally form a cycloalkyl or heterocycloalkyl optionally substituted with one or more R_{f};
each of m, n, and i, independently, is 0, 1, 2, 3, 4, 5, or 6; and
j is 0, 1, 2, 3, 4, 5, or 6.

2. The compound according to claim 1 or an N-oxide thereof, or a pharmaceutically acceptable salt, solvate, polymorph, tautomer, stereoisomer, an isotopic form, or a prodrug thereof, wherein the compound is represented by Formula (2): 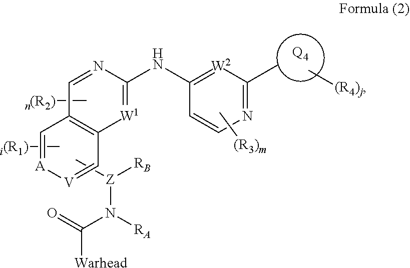
wherein
Warhead is 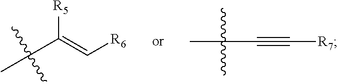
and
each of R₅, R₆, or R₇, independently, is H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, heteroaryl, each of which is independently optionally substituted with one or more R_{d}.

3. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (3):

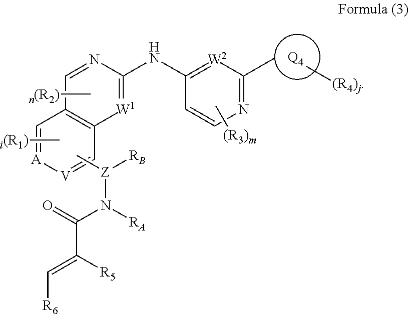

4. The compound according to claim 3 or an N-oxide thereof, or a pharmaceutically acceptable salt, solvate, polymorph, tautomer, stereoisomer, an isotopic form, or a prodrug thereof, wherein the compound is represented by Formula (4): 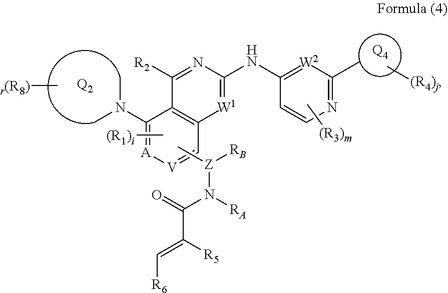
wherein
Q₂, is a heterocycloalkyl, heterocycloalkenyl, spiro-heterocyclic, fused-heterocyclic, or bridged-heterocyclic, each of which is independently optionally substituted with one or more R_{d};
each of R₈, independently, is absent, H, D, alkyl, alkenyl, alkynyl, halo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, heteroaryl, each of which is independently optionally substituted with one or more Rₐ;
r is 0, 1, 2, 3, 4, 5, or 6; and
i is 0, 1, or 2.

5. The compound according to claim 4 or an N-oxide thereof, or a pharmaceutically acceptable salt, solvate, polymorph, tautomer, stereoisomer, an isotopic form, or a prodrug thereof, wherein the compound is represented by Formula (5):

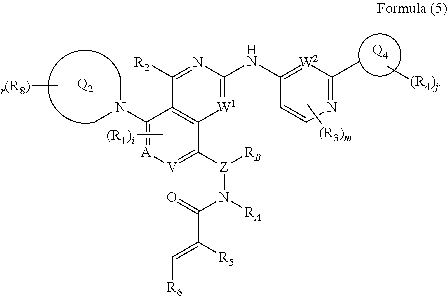

6. The compound according to claim 5 or an N-oxide thereof, or a pharmaceutically acceptable salt, solvate, polymorph, tautomer, stereoisomer, an isotopic form, or a prodrug thereof, wherein the compound is represented by Formula (6): 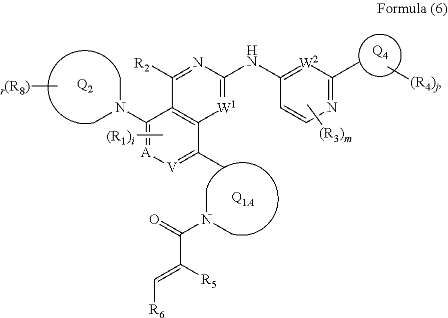
wherein
Q_{1A} is a heterocycloalkyl, heterocycloalkenyl, spiro-heterocyclic, fused-heterocyclic, or bridged-heterocyclic, each of which is independently optionally substituted with one or more R_{d}.

7. The compound according to claim 4 or an N-oxide thereof, or a pharmaceutically acceptable salt, solvate, polymorph, tautomer, stereoisomer, an isotopic form, or a prodrug thereof, wherein the compound is represented by Formula (7): 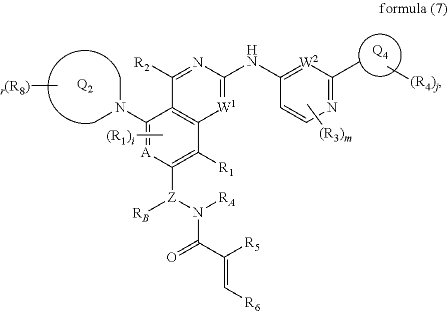
wherein
i is 0 or 1.

8. The compound according to claim 7 or an N-oxide thereof, or a pharmaceutically acceptable salt, solvate, polymorph, tautomer, stereoisomer, an isotopic form, or a prodrug thereof, wherein the compound is represented by Formula (8): 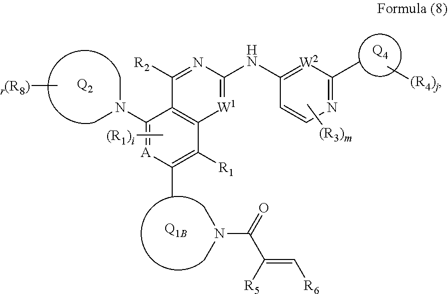
wherein
Q_{1B} is a heterocycloalkyl, heterocycloalkenyl, spiro-heterocyclic, fused-heterocyclic, or bridged-heterocyclic, each of which is independently optionally substituted with one or more R_{d}.

9. A pharmaceutical composition comprising a compound of Formula (1) or an N-oxide thereof as defined in any one of claims 1-8, or a pharmaceutically acceptable salt, solvate, polymorph, tautomer, stereoisomer, an isotopic form, or a prodrug of said compound of Formula (1) or an N-oxide thereof, and a pharmaceutically acceptable diluent or carrier.

10. A method of treating a neoplastic disease, autoimmune disease, and inflammatory disorder, comprising administering to a subject in need thereof an effective amount of a compound of Formula (1) or an N-oxide thereof as defined in any one of claims 1-8, or a pharmaceutically acceptable salt, solvate, polymorph, tautomer, stereoisomer, an isotopic form, or a prodrug of said compound of Formula (1) or an N-oxide thereof.
